SEQUENCE LISTING (23 sequences)

SEQ ID NO: 1 - protein, 40 aa, Artificial Sequence
  Synthetic
  misc_feature (27)..(27): Xaa can be any naturally occurring amino acid
TVGGYTCQRNSVPVQVSLNSPGVYTKXCMKVDWTQDTTAN

SEQ ID NO: 2 - protein, 39 aa, Artificial Sequence
  Synthetic
IVGGRRARPIAWPBMVSLQLPDAFAPVAQFVNWIDSIIQ

SEQ ID NO: 3 - protein, 41 aa, Artificial Sequence
  Synthetic
  misc_feature (27)..(27): Xaa can be any naturally occurring amino acid
TYGGHBAKRHSRPYMAYLQIDRAHTKXSTPLSWIKKTMKKS

SEQ ID NO: 4 - protein, 41 aa, Artificial Sequence
  Synthetic
TVEGSDAETCMSPMQVMLFRYGFYTHVFRLKKWTQKVTDQF

SEQ ID NO: 5 - protein, 39 aa, Artificial Sequence
  Synthetic
  misc_feature (13)..(13): Xaa can be any naturally occurring amino acid
ITNGAYDGQABVXVGMAFPAGFDRITSQLMWIRQHTGLY

SEQ ID NO: 6 - protein, 41 aa, Artificial Sequence
  Synthetic
VNGNFDCGVRGWPFHVGLYDCGVNTLTGLYSGWIQQQLQLF

SEQ ID NO: 7 - protein, 41 aa, Artificial Sequence
  Synthetic
ITGGYRAKPYTIIYLVGIVYPGVHIRVSDDIKWIKDVSGVG

SEQ ID NO: 8 - protein, 40 aa, Artificial Sequence
  Synthetic
SLREEPBIITVTLKKQGMGLSIVTRTSSVVTLEVAKQGAI

SEQ ID NO: 9 - protein, 39 aa, Artificial Sequence
  Synthetic
MKEPEIITVTLKKQNGMGLSIVTRTSSVVTLEVAKQGAL

SEQ ID NO: 10 - protein, 40 aa, Artificial Sequence
  Synthetic
SSGLELFPVELEKDEDGLGISTIRNTKGNVRFVIGREKPS

SEQ ID NO: 11 - protein, 41 aa, Artificial Sequence
  Synthetic
FQSMTVVEIKLFKGPKGLGFSTAKNTSSEVVYLKVGKPTTI

SEQ ID NO: 12 - protein, 39 aa, Artificial Sequence
  Synthetic
EVIWEQYTWTLQKDSKGFGIAVSRKSGKIAAIVVKPRKV

SEQ ID NO: 13 - protein, 39 aa, Artificial Sequence
  Synthetic
LQGGDEKKVNLVLGDGSLGLTIRKSSRHLILTVKDVGRL

SEQ ID NO: 14 - protein, 40 aa, Artificial Sequence
  Synthetic
SSGPGLRELCIQKAPGRLGISIRRSVGDTLTVLVCDGFES

SEQ ID NO: 15 - protein, 40 aa, Artificial Sequence
  Synthetic
GQDRDYPTVDMEKGAKGFGPSIRKSGGRRVRLLLKRGTGS

SEQ ID NO: 16 - protein, 40 aa, Artificial Sequence
  Synthetic
EGSSGRHVACLARSERGLGFSTATAASPTIALLLEREAGS

SEQ ID NO: 17 - protein, 39 aa, Artificial Sequence
  Synthetic
IAGMDVRLLRIKKEGSLDLALEQKAWNWIDLVVAVCPPK

SEQ ID NO: 18 - protein, 39 aa, Artificial Sequence
  Synthetic
DRDPAFRVITVTKETGLGLKILTRAKLPWEIAFIRSGPS

SEQ ID NO: 19 - DNA, 20 bp, Artificial Sequence
  Synthetic
agcgatctcggtgacgatgg

SEQ ID NO: 20 - DNA, 23 bp, Artificial Sequence
  Synthetic
cattaacgatgcaagtctcgtgg

SEQ ID NO: 21 - DNA, 59 bp, Artificial Sequence
  Synthetic
  misc_feature (34)..(39): n is a, c, g, or t
tgactggagttcagacgtgtgctcttccgatctnnnnnnacgactcactatagggagac

SEQ ID NO: 22 - DNA, 58 bp, Artificial Sequence
  Synthetic
  misc_feature (33)..(38): n is a, c, g, or t
cactctttccctacacgacgctcttccgatctnnnnnntgactagtcattattagtgg

SEQ ID NO: 23 - DNA, 52 bp, Artificial Sequence
  Synthetic
caagcagaagacggcatacgagatcgagtaatgtgactggagttcagacgtg

(19) United States
(12) Patent Application Publication
(10) Pub. No.: US 2022/0348903 A1
(43) Pub. Date: Nov. 3, 2022

(54) METHOD AND APPARATUS USING MACHINE LEARNING FOR EVOLUTIONARY DATA-DRIVEN DESIGN OF PROTEINS AND OTHER SEQUENCE DEFINED BIOMOLECULES

(71) Applicant: THE UNIVERSITY OF CHICAGO, Chicago, IL (US)

(72) Inventors: Rama Ranganathan, Chicago, IL (US); Andrew Ferguson, Chicago, IL (US)

(21) Appl. No.: 17/642,582

(22) PCT Filed: Sep. 11, 2020
(86) PCT No.: PCT/US20/50466

§ 371 Date: Mar. 11, 2022

Related U.S. Application Data
(60) Provisional application No. 63/020,083, filed on May 5, 2020.
(60) Provisional application No. 62/900,420, filed on Sep. 13, 2019.

(51) Int. Cl. C12N 15/10; G16B 35/10; G16B 40/30

(52) U.S. Cl. CPC C12N 15/1058; G16B 35/10; G16B 40/30

(57) ABSTRACT

A method and apparatus are provided for designing sequence-defined biomolecules, such as proteins using a data-driven, evolution-based process. To design proteins, an iterative method founded on a combination of an unsupervised sequence-based model with a supervised functionality-based model can select candidate amino acid sequences that are likely to have a desired functionality. Feedback from measuring the candidate proteins using a high-throughput gene-synthesis and a protein screening process is used to refine and improve the models guiding the candidate selection to the most promising regions of the very large amino acid sequence search space.

### RELATED APPLICATIONS

This application claims the benefit of U.S. Provisional Application No. 63/020,083 (filed on May 5, 2020); and U.S. Provisional Application No. 62/900,420 (filed on Sep. 13, 2019). The entirety of each of the foregoing provisional applications is incorporated by reference herein.

### FIELD

This disclosure relates to a data-driven, evolution-based method for designing sequence-defined molecules, such as proteins, and, more particularly, relates to an iterative method that combines an unsupervised sequence model with a supervised functionality model to design proteins to have a desired functionality.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Proteins are molecular machines that participate in a variety of biological processes, including those that are essential for life. For example, they have the ability to catalyze microsecond biochemical reactions in the body that would otherwise take years. Proteins are involved in transport (the blood protein hemoglobin transports oxygen from the lungs to the tissues), motility (flagella provide sperm motility), information processing (proteins make up signal transduction pathways in cells), and cell regulation cues (like the hormone insulin) fundamental for normal bodily function. Antibodies that provide host immunity are proteins, as are the molecular motors, like kinesin and myosin, responsible for muscle contraction and intercellular transport. When light reaches the eyes, a membrane protein in the eyes called rhodopsin senses the incident photons and, in turn, activates a cascade of downstream proteins to eventually tell the brain what is seen by the eyes. Proteins thus perform highly diverse and specialized functions.

Although proteins exhibit a remarkable array of properties, all proteins are polymers built from only 20 units called amino acids. Every protein is a linear arrangement of amino acids, referred to as the protein sequence. In its native state, the protein molecule itself twists, turns, and folds to generally form an irregular three-dimensional globule. The precise three-dimensional arrangement of the amino acids, called the protein structure, and the interactions between the amino acids give rise to the function of the protein. A model for protein function can be used to derive the functional properties identifying all the atomic interactions in the protein molecule (i.e., its energetic architecture). Two independent approaches—one based on protein structure and the other based on evolutionary statistics—exist to understand the energetic architecture of proteins.

The structure-guided view has value. For example, the role of binding site residues can be tested by mutagenesis, based on the idea that if critical amino acids are substituted to a residue with less average functional activity (e.g. alanine), the protein is predicted to display a decrease in its ability to function. For example, using this approach the importance of amino acids that comprise the interface of protein and ligand has been demonstrated.

However, the principle of spatial proximity described above does not fully capture the determinants of biochemical function. For example, amino acids could interact in complex cooperative ways in the structure to influence binding site function even from a distance, and the structure alone provides no general model for understanding how such cooperativities are arranged. Accordingly, other approaches to protein design are desired. In particular, the optimization space for protein design is too large and complicated to be tractable using only a structure-based approach.

The goal of protein design is to identify novel molecules that have certain desirable properties. This can be viewed as an optimization problem, in which a search is performed for the proteins that maximize given quantitative desiderata. However, optimization in protein space is extremely challenging because the search space is large, discrete, and mostly filled with unstructured, non-functional sequences. Making and testing new proteins are costly and time-consuming, and the number of potential candidates is overwhelmingly large.

Accordingly, improved techniques are desired to more efficiently and robustly search for protein that are optimized according to the given quantitative desiderata.

### BRIEF DESCRIPTION OF THE DRAWINGS

A more complete understanding of this disclosure is provided by reference to the following detailed description when considered in connection with the accompanying drawings, wherein:

FIG. 1A shows a flow diagram of a method 10 for designing sequence-defined molecules, according to one implementation;

FIG. 1B shows a more detailed flow diagram of the method 10 as implemented for proteins, according to one implementation;

FIG. 1C shows a schematic of a computational model, according to one implementation, for generating synthetic sequences for a sequence-defined molecule;

FIG. 1D shows a schematic of a method 10, according to one implementation, for data-driven design of a sequence-defined molecules;

FIG. 1E shows a diagram illustrating example designed or candidate proteins resulting from the systems and/or methods described herein, where such proteins may be provided (e.g., as end products) in one or more alternative forms and/or applied to various industries;

FIG. 2 shows an example of a path traversed in an iterative search to design proteins, according to one implementation;

FIG. 3A shows a flow diagram of another implementation of method 10 in an implementation having an unsupervised-learning part and a supervised-learning part, according to one implementation;

FIG. 3B shows a flow diagram of an iterative loop in the supervised-learning part of method 10, according to one implementation;

FIG. 3C shows a schematic diagram of method 10 represented as in terms of the unsupervised-learning part and the supervised-learning part, according to one implementation;

FIG. 4 shows a schematic diagram of a variational auto-encoder (VAE), according to one implementation;

FIG. 5 shows a schematic diagram of a VAE being used in combination with a function defined by Gaussian process regression to find Pareto optimal candidate amino acid sequences, according to one implementation;

FIG. 6A shows a schematic diagram for a first implementation of gene synthesis, according to one implementation;

FIG. 6B shows a schematic diagram for a second implementation of gene synthesis, according to one implementation;

FIG. 7 shows a mapping between codons in a genetic sequences and amino acids;

FIG. 8A shows a schematic diagram of a method 10 using a direct coupling analysis (DCA) model for the sequence model, according to one implementation;

FIG. 8B shows a portion of the shikimate pathway in bacteria and fungi, leading to the biosynthesis of the aromatic amino acids tyrosine and phenylalanine;

FIG. 8C shows an atomic structure of E. coli chorismate mutase (CM) including a dimer (e.g., items 800c1, 800c2, and 800c3) with two functional active sites;

FIG. 9A shows first order statistics of sequences sampled from a Boltzmann machine direct coupling analysis (bmDCA) model (i.e., a recapitulating the empirical first order statistics MSA), according to one implementation;

FIG. 9B shows second order statistics of sequences sampled from the bmDCA model recapitulating the empirical second order statistics MSA, according to one implementation;

FIG. 9C shows third order statistics of sequences sampled from the bmDCA model recapitulating the empirical third order statistics MSA, according to one implementation;

FIG. 9D shows the top two principal components of the distance matrix between all natural CM sequences in the MSA (circles shaded as, e.g., item 900d1) and the sequences derived from the bmDCA model (circles shaded as, e.g., item 900d2), according to one implementation;

FIG. 9E shows a quantitative high-throughput functional assay for CM in which libraries of CM variants are expressed in an E. coli strain deficient for chorismate mutase, grown as a mixed population under selective conditions, and then subject to next generation sequencing to count the frequency of each CM allele in the input and selection populations;

FIG. 9F shows a plot of an approximately linear relations between a computed relative enrichment (“r.e.”) and a catalytic power, ln(kc/Km), over a roughly five log-order range;

FIG. 10A shows a histogram plot of number of natural CM sequences as a function of statistical energy, according to one implementation;

FIG. 10B shows a histogram plot as a function of r.e. score for natural CM sequences, according to one implementation;

FIG. 10C shows a histogram plot as a function of statistical energy for the bmDCA generated sequences at temperature T=0.33, according to one implementation;

FIG. 10D shows a histogram plot as a function of statistical energy for the bmDCA generated sequences at temperature T=0.66, according to one implementation;

FIG. 10E shows a histogram plot as a function of statistical energy for the bmDCA generated sequences at temperature T=1.0, according to one implementation;

FIG. 10F shows a histogram plot as a function of r.e. score for the bmDCA generated sequences at temperature T=0.33, according to one implementation;

FIG. 10G shows a histogram plot as a function of r.e. score for the bmDCA generated sequences at temperature T=0.66, according to one implementation;

FIG. 10H shows a histogram plot as a function of r.e. score for the bmDCA generated sequences at temperature T=1.0, according to one implementation;

FIG. 10I shows a histogram plot as a function of statistical energy for sequences generated using only first-order statistics, according to one implementation;

FIG. 10J shows a histogram plot as a function of an r.e. score for sequences generated using only first-order statistics, according to one implementation;

FIG. 11A shows a scatterplot of all synthetic CM sequences, showing the relationship between bmDCA statistical energy and catalytic function, with functional sequences (bars shaded as, e.g., item 1100a1) and non-functional sequences (bars shaded as, e.g., item 1100a2);

FIG. 11B shows a scatterplot as a function of the top two principal components of sequence variation for natural CM sequences in the MSA, with functional sequences (circles or items shaded as, e.g., item 1100b1) and non-functional sequences (circles or items shaded as, e.g., item 1100b2).

FIG. 11C shows a scatterplot as a function of the top two principal components of sequence variation for sequences derived from the bmDCA model, with functional sequences (circles or items shaded as, e.g., item 1100c1) and non-functional sequences (circles or items shaded as, e.g., item 1100c2);

FIG. 11D shows a histogram plot of the number of synthetic sequences with EDCA <40, either without an extra statistical condition derived from the pattern of functional complementation of natural CM sequences (P(x=116));

FIG. 11E shows a histogram plot of the number of synthetic sequences with EDCA <40, either with an extra statistical condition derived from the pattern of functional complementation of natural CM sequences (P(x=116));

FIG. 11F shows a structure of E. coli CM, with positions contributing most to low statistical energy (spheres shaded as, e.g., item 1100f2), and positions contributing to E. coli specific function (spheres shaded as, e.g., item 1100f1);

FIG. 12 shows a schematic diagram of a protein optimization system, according to one implementation;

FIG. 13 shows a flow diagram of a method for training a deep learning (DL) network, according to one implementation; and

FIG. 14 shows an example of an artificial neural network, according to one implementation.

The Figures described herein depict various aspects of the system and methods disclosed therein. It should be understood that each Figure depicts an embodiment of a particular aspect of the disclosed systems and methods, and that each of the Figures is intended to accord with a possible embodiment thereof. Further, wherever possible, the description herein refers to the reference numerals included in the Figures herein, in which features depicted in multiple Figures are designated with consistent reference numerals.

### SUMMARY

According to aspects of one embodiment, there is provided a method of designing proteins to have a desired functionality. The method includes steps of (i) determining candidate amino acid sequences of synthetic proteins using a machine-learning model that has been trained to learn implicit patterns in a training dataset amino acid sequences of proteins, the machine-learning model expressing the learned implicit patterns in a latent space; and (ii) performing an iterative loop. Each iteration of the loop comprises, includes steps of (i) synthesizing candidate genes and producing candidate proteins corresponding to the respective candidate amino acid sequences, each of the candidate genes coding for the corresponding candidate amino acid sequence, (ii) evaluating a degree to which the candidate proteins respectively exhibit a desired functionality by measuring values of the candidate proteins using one or more assays, and (iii), when one or more stopping criteria of the iterative loop have not been satisfied, calculating, from the measured values, a fitness function in the latent space, and selecting, using a combination of the fitness function together with the machine-learning model, candidate amino acid sequences for a subsequent iteration.

According to aspects of another embodiment, there is provided a system for designing proteins to have a desired functionality. The system includes (i) a gene synthesis system, (ii) an assay system, and (iii) processing circuitry. The gene synthesis system is configured to synthesize candidate genes corresponding to respective candidate gene sequences that code for candidate amino acid sequences. The assay system is configured to measure values of candidate proteins corresponding to respective candidate amino acid sequences, the measured values providing indicia of a desired functionality. The processing circuitry is configured to (i) determine the candidate amino acid sequences of synthetic proteins using a machine-learning model that has been trained to learn implicit patterns in a training dataset of proteins, the machine-learning model expressing the learned implicit patterns in a latent space, and (ii) perform an iterative loop. Each iteration of the loop includes the steps of (a) sending the candidate amino acid sequences to be synthesized, (b) receiving the measured values generated by measuring the candidate proteins using one or more assays, and (c), when one or more stopping criteria of the iterative loop have not been satisfied, calculating, from the measured values, a fitness function in the latent space, and selecting, using a combination of the fitness function together with the machine-learning model, candidate amino acid sequences for a subsequent iteration.

According to aspects of a third embodiment, there is provided a non-transitory computer readable storage medium including executable instructions, wherein the instructions, when executed by circuitry, cause the circuitry to perform the steps of (i) determining candidate amino acid sequences of synthetic proteins using a machine-learning model that has been trained to learn implicit patterns in a training dataset amino acid sequences of proteins, the machine-learning model expressing the learned implicit patterns in a latent space; and (ii) performing an iterative loop. Each iteration of the loop comprises, includes steps of (i) synthesizing candidate genes and producing candidate proteins corresponding to the respective candidate amino acid sequences, each of the candidate genes coding for the corresponding candidate amino acid sequence, (ii) evaluating a degree to which the candidate proteins respectively exhibit a desired functionality by measuring values of the candidate proteins using one or more assays, and (iii), when one or more stopping criteria of the iterative loop have not been satisfied, calculating, from the measured values, a fitness function in the latent space, and selecting, using a combination of the fitness function together with the machine-learning model, candidate amino acid sequences for a subsequent iteration.

According to aspects of a fourth embodiment, there is provided a method of designing proteins to have a desired functionality. The method includes steps of (i) determining candidate genetic sequences of biomolecules, which are sequence defined, the candidate genetic sequences being generated using a machine-learning model that has been trained to learn implicit patterns in a training dataset of sequence-defined biomolecules, the machine-learning model expressing the learned implicit patterns in a latent space; and (ii) performing an iterative loop. Each iteration of the loop comprises, includes steps of (i) synthesizing candidate genes corresponding to the respective candidate genetic sequences, each of the candidate genes coding for the corresponding candidate biomolecule, (ii) evaluating a degree to which the candidate biomolecules respectively exhibit a desired functionality by measuring values of the candidate biomolecules using one or more assays, and (iii), when one or more stopping criteria of the iterative loop have not been satisfied, calculating, from the measured values, a fitness function in the latent space, and selecting, using a combination of the fitness function together with the machine-learning model, candidate genetic sequences for a subsequent iteration.

In accordance with the above, and with the disclosure herein, the present disclosure includes improvements in computer functionality or in improvements to other technologies at least because the inventions disclosed herein recite, e.g., an underlying computer or machine that is continuously improved by updating a machine-learning model to more accurately predict or identify design or candidate proteins. That is, the present disclosure describes improvements in the functioning of the computer itself or “any other technology or technical field” because the prediction or evaluation generated by the underlying computing device improves overtime as the machine-learning model of the computing device is further trained (over various iterative loops) to better identify or predict candidate or design proteins. This improves over the prior art at least because prior art systems were incapable of being improved without manual coding or development by a human developer.

The present disclosure relates to improvement to other technologies or technical fields at least because the present disclosure describes the use of artificial intelligence (e.g., machine learning models) applied to designing proteins having a desired functionality.

The present disclosure includes applying features herein with, or by use of, a particular machine, e.g., a micro-fluidics apparatus for measuring fluorescence of cells corresponding to candidate or design proteins identified by the artificial intelligence based systems and methods described herein.

The present disclosure includes effecting a transformation or reduction of a particular article to a different state or thing, e.g., transforming or reducing a candidate or design protein, as identified by the artificial intelligence based systems and methods described herein, into cells that may be used to generate, produce, or otherwise catalyze an end product.

The present disclosure includes specific features other than what is well-understood, routine, conventional activity in the field, or adding unconventional steps that confine the claim to a particular useful application, e.g., including systems and methods of designing proteins having a desired functionality, e.g., for the development, manufacture, or creation of real-world products.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

The methods described herein use data-driven and evolution-based approaches that leverage statistical genomics, machine learning, and artificial intelligence techniques to learn implicit patterns relating amino acid sequences to protein structure, function, and evolvability, thereby overcoming the limitations and challenges of previous protein design methods. In one embodiment, proteins are desired to have a desired functionality by first training a machine-learning model based on sequence information of proteins in a training dataset (e.g., a database of homologous proteins), and use the trained machine-learning model to generate candidate amino acid sequences.

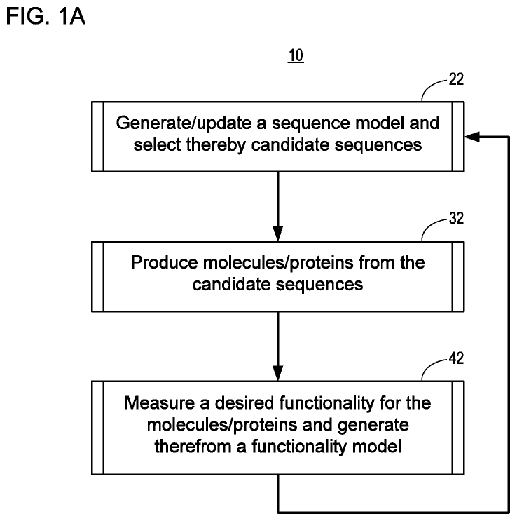

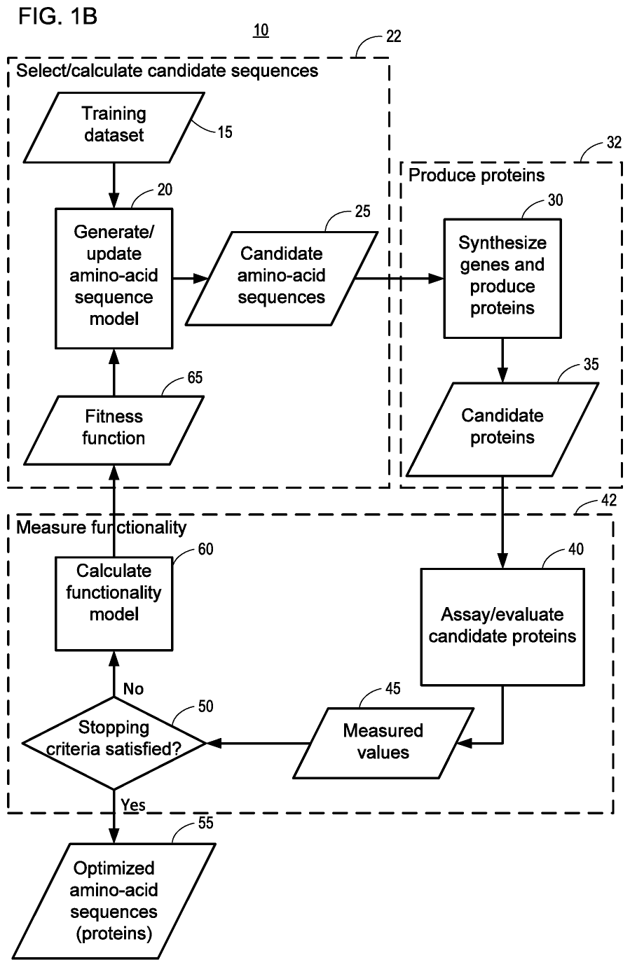

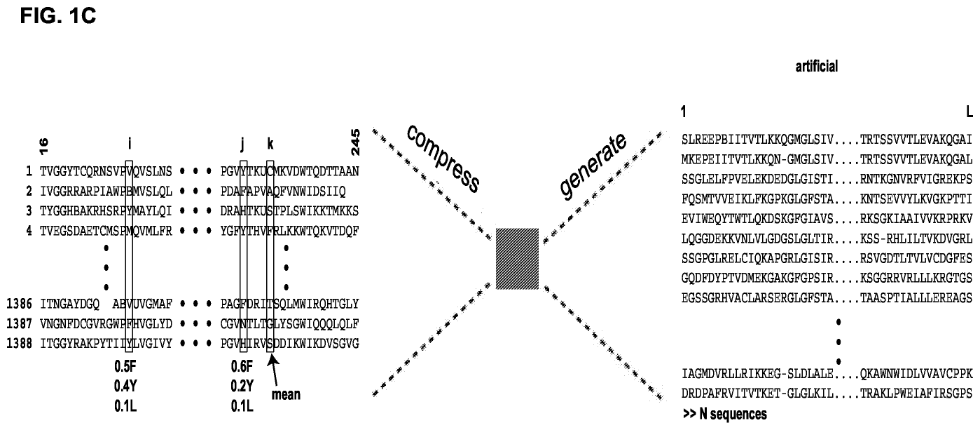

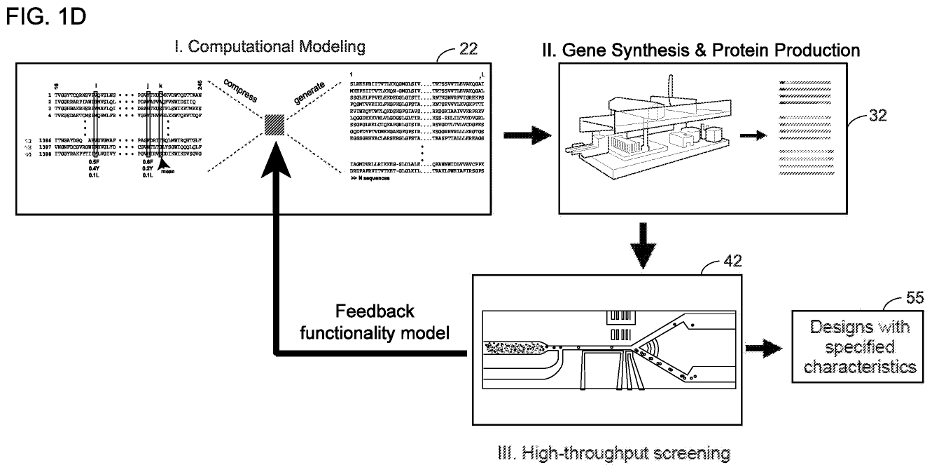

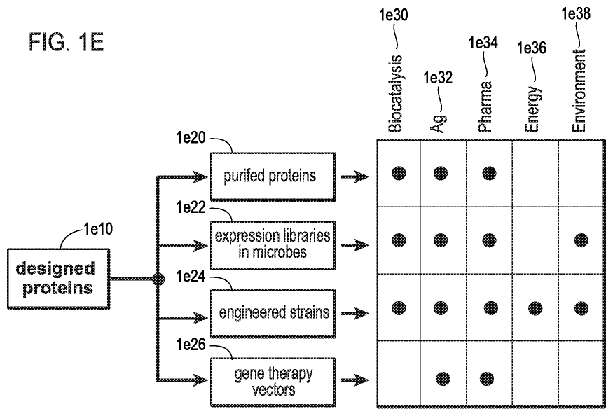

Next, an iterative process is performed to select ever better candidate amino acid sequences. This iterative process includes producing proteins for the candidate amino acid sequences and assaying them to measure their functionality. Using the measured values from the assays, a functionality landscape (e.g., a functionality-based model) is generated to predict which candidate sequences are most likely to exhibit the desired functionality. Using a fitness function based on the functionality landscape together with the machine-learning new candidate sequence can be determined having better functionality than the first. The iterative process is then repeated with each iteration yielding new candidate sequence that are better than the previous iteration, until stopping criteria are reached and the final, optimized amino acid sequences are output as the designed proteins. This iterative process is illustrated in FIG. 1A, for example.

The methods described herein use both information provided by the sequences themselves (i.e., a sequence-based model) and functionality information (i.e., a functionality-based model) that is provided by producing and evaluating the proteins using assays that measure the functionality of the proteins. The combination of the sequence-based model together with the functionality-based model is referred to as a protein model.

The sequence-based model is generally an unsupervised machine-learning model that receives as training data the amino acid sequences from an ensemble of proteins. Accordingly, the sequence-based model is referred to as a machine-learning model, an unsupervised model, an amino acid sequence-based model, or any combination thereof.

Generally, the functionality-based model is a supervised model because it generated/trained using information from additional measurements (i.e., supervision). The functionality-based model is often expressed as a fitness function and/or a functionality landscape. For example, when multi-objective multi-dimensional optimization is used in the design process, a functionality landscape is one of the components in a fitness function that identifies which amino acid sequences are good candidates when designing for the desired functionality. Accordingly, the functionality-based model can be referred to as a supervised model, a fitness function, a functionality landscape, a functionality-based model, a functionality model, or any combination thereof. The functionality-based model is generated via machine learning and can be a machine-learning model. However, the term “machine-learning model” as used herein generally refers to the sequence-based model, unless context clearly indicates otherwise.

As discussed above, the goal of protein design is to identify novel proteins that have certain desirable properties. This can be viewed as an optimization problem, in which a search is performed for the proteins that maximize given quantitative desiderata. However, optimization in protein space is extremely challenging because the search space is large, discrete, and unstructured (e.g., in the context of data mining, an amino acid sequence is classified as unstructured data, with all the challenges that classification entails). Making and testing new proteins are costly and time-consuming, and the number of potential candidates is overwhelmingly large.

For example, the number of possible amino acid sequences of length N is 20^{N}, and most of this space of sequences is occupied by non-folding, non-functional molecules. Directed evolution provides one approach to search this space for structured, functional molecules by rounds of mutation and selection for function, but represents only a very local exploration of the sequence space around particular natural sequences. Accordingly, formal rules are needed to overcome these limitations of directed evolution in order to guide a search that can explore the vastly larger combinatorial complexity of possible functional sequences.

One set of rules come from physical models for the fundamental forces between atoms—this is physics-based design. But these methods are limited (i) by inaccuracies in the force fields, (ii) by imposition of ad hoc constraints to manage the combinatorial complexity of the search, and (iii) by the philosophy that globally-optimized sequences are the best. Accordingly, even if sequences generated via physics-based design can form highly stable structures, the evidence so far is that without directed evolution such sequences suffer from poor functional performance.

In contrast to physics-based design and directed-evolution approaches, the methods described herein use evolutionary data-driven statistical models. These kinds of models provide a distinct approach to obtain rules for searching the sequence space that does not depend on knowledge of the underlying physical forces or knowledge of the mechanisms of folding, function, or evolution. Furthermore, these models do not require three-dimensional structures as starting points. Instead, these models capture the pattern of statistical constraints that define the natural ensemble of protein sequences, and by so doing indirectly capture the physics of protein folding and function. Thus, rather than globally optimizing the protein for stability, the methods described herein optimize whatever constraints that nature has selected through the history of evolution, enabling the sequence space to be searched for functional proteins with vastly greater yield and depth than previous methods.

To address the above-identified challenges with previous protein design methods, the systems and methods described herein use a data-driven evolution-based iterative approach to identify/select the most promising amino acid sequences for the desired protein/enzyme functionality.

The data-driven aspect of this approach arises in part from using data to train a combination of models, including a sequence-based model and a functionality-based model. The sequence-based model (also referred to as an unsupervised model) can be a machine-learning model that is trained to express statistical properties and implicit patterns that are learned from a training dataset (e.g., a multiple sequence alignment (MSA) of homologous proteins). The functionality-based model (also referred to as a supervised model) incorporates feedback measured from assays of synthesized proteins that are based on amino acid sequences that were identified as promising candidates in previous iterations. The assays are designed to provide indicia regarding desired protein functionality. For example, a desired functionality (e.g., binding, allostery, or catalysis) can be quantitatively related to organismal growth rate, gene expression, or changes in optical properties such as light absorbance or fluorescence in various particular assays.

The evolution-based aspect of this approach arises in part from using an iterative feedback loop to identify trends for best performing amino acid sequences to perform selection for improved amino acid sequences based on the trends (i.e., in silico mutagenesis).

Further, in certain implementations, the training dataset will consist of homologs. Thus, the implicit patterns learned by the sequence-based model will include sequence patterns for the desired functionality that have been learned over time through biological evolution.

Thus, the methods described herein address the challenge of rapidly identifying synthetic proteins that are likely to exhibit a desired functionality through an iterative cycle of using data-driven computational models of proteins to generate candidate amino acid sequences, then physically producing and evaluating the candidate proteins in a lab using quantitative assays, and finally using information from the assays to improve the computational models to generate candidate amino acid sequences further optimized for the desired functionality in the next iteration. Thus, a feedback loop is established to iteratively optimize amino acid sequences for a desired functionality.

In particular, the protein model can be broken down into two parts: a supervised model and an unsupervised model. In a first iteration of an iterative loop, the initial candidate amino acid sequences can be determined using only the unsupervised model. Then in subsequent iterations, both the supervised model and the unsupervised model can be used to generate the candidate amino acid sequences in subsequent iterations.

Referring now to the drawings, wherein like reference numerals designate identical or corresponding parts throughout the several views, FIGS. 1A and 1B show flow diagrams of a method 10 using a combination of a sequence model and a functionality model to design synthetic proteins.

In process 22 of method 10, a sequence-based model (also referred to an unsupervised machine-learning model or more simply an unsupervised model) is trained using a training dataset of amino acid sequences to generate new amino acid sequences having similar statistical structure and/or patterns as those learned from the training dataset. More generally, the methods described herein apply to any sequence-defined molecules including biomolecules—not just proteins. For example, a nucleic acid, such as messenger RNA or micro RNA, can be designed to have desired functionality. In this case the candidate sequences would be nucleotide sequences rather than amino acid sequences; otherwise method 10 would be the same only with minor, straightforward variations, as would be understood by a person of ordinary skill in the art. As another example, a polymer defined by sequences of monomers could be designed to have desired functionality using the method described herein. For concreteness, the methods and systems described herein are illustrated using the non-limiting example of protein design, but the methods and systems described herein are general and include the design of all sequence-defined molecules.

In process 32 of method 10, for the candidate sequences that were determined in process 22, sequence-defined molecules (e.g., proteins) are produced physically in a laboratory.

In process 42 of method 10, the sequence-defined molecules are assayed to measure how much they exhibit the desired functionality. These measured values are then incorporated into a functionality model, which is then used at process 22 in the next iteration to select new, better candidates based on the measured feedback arising from process 42.

Over time, method 10 will converge to candidate sequences satisfying predefined stopping criteria (e.g., the degree of desired functionality corresponding to the candidate sequences exceeding a predefined threshold, or the rate of increase in the desired functionality from iteration to iteration has slowed below another predefined threshold). Then, method 10 will output one or more of the optimized candidate sequences as the designed proteins. As used herein, the terms “candidate protein” and “designed protein” may be used interchangeably, where a designed protein indicates an output of a candidate protein for a given iteration of the systems and/or methods described herein, e.g., including an iteration of an iterative loop and/or machine-learning model as described herein.

Returning to process 22, in non-limiting examples provided herein, the sequence model is illustrated as being restricted Boltzmann machines (RBMs), variational auto-encoders (VAEs), generative-adversarial networks (GANs), statistical coupling analysis (SCA), and direct coupling analysis (DCA).

The first three types of sequence models (i.e., RBMs, VAEs, and GANs) are types of artificial neural networks (ANNs), and are often referred to as “generative methods” because they map the inputs at the visible nodes of the ANNs to a hidden layer of nodes that has a reduced dimension compared to the visible layer, providing an informational bottleneck in which information is compressed and implicit patterns are captured. This hidden layer of nodes defines a latent space, and the mapping from the visible layer to the hidden space is referred to herein as “encoding,” with the reverse process of mapping points in the latent space back to the original space of the amino acid sequences being referred to herein as “decoding” or “generating.” The learned patterns resulting from compressing to a reduced subspace result in the random selection of points in the latent space followed by decoding to points resulting in amino acid sequences having similar patterns to those in the training dataset.

For example, a VAE that is trained using training images of faces can be used to generate an image of that is recognizable as being a face, albeit a different face than those in the training images (i.e., the VAE learns the characteristics of faces in general, and can thereafter be used to generate new faces images having the learned characteristics). If it was desired that the VAE be further trained not only to recognize faces but faces having desired characteristics (e.g., beautiful faces), a user could go through the faces generated by the VAE to tag the figures according to the desired characteristics. Then, supervised learning could be performed by using the indicia of the desired characteristics to learn the patterns for beautiful faces or some other desired characteristic. For example, a VAE trained using only beautiful faces would learn the patterns for beautiful faces. Similar principles can be applied for learning the patterns of amino acids sequences for a particular type of protein (e.g., homologous proteins) to generate new candidates, and then to further learn a subset of candidate amino acids sequences exhibiting a desired functionality through supervised learning techniques.

Returning to the unsupervised sequence models of process 22, the last two types of models (i.e., SCA and DCA) are often referred to as “statistical methods.” These statistical methods also generate candidate amino acid sequences, but do so by learning the statistical properties of the amino acid sequences in the training data set (e.g., the first and second order statistics) and then selecting candidate amino acid sequences conforming to the learned statistical model. That is, like the generative methods, the statistical methods generate candidate amino acid sequences, but the statistical methods do not use neural networks to map between a sequence space and a latent space. Rather, these statistical methods operate within the domain of the sequence space to learn the patterns for subspace within the sequence space (similar to but different from the latent spaces in the generative methods) that is likely to produce proteins similar to those in a training dataset.

That is, the unsupervised sequence models narrow the search by restricting the search space from the “overwhelmingly large” number of all possible candidate amino acid sequences to a much smaller and more manageable subset of sequences that are likely to exhibit the desired functionality/characteristics. Then, from this subset a few amino acid sequences (e.g., on the order of 1,000) are selected as candidate amino acid sequences to be produced in a lab and evaluated to provide measured data/values for supervised learning. It is to be understood that the selected candidate amino acid sequences to be produced can comprise various numbers and counts including by way of non-limiting example, at least 100 amino acid sequences, at least 500 amino acid sequences, at least 1000 amino acid sequences, at least 1500 amino acid sequences, etc.

Considering now the supervised functionality models in process 42, in certain implementations, the supervised model can be thought of as a functionality landscape on the latent space, in which the peaks correspond to regions in the latent space that are likely to produce candidate amino acid sequences that exhibit more of the desired functionality, and valleys are unlikely to produce good candidate amino acid sequences. For example, this functionality landscape is generated by performing regression analysis on the measured values, which were generated by performing assays on the candidate amino acid sequences to measure the desired functionality (e.g., catalysis, binding, or allostery). In non-limiting examples provided herein, the supervised functionality model is illustrated as being generated by fitting the measured values to a functionality landscaped using a multivariable linear regression, a support vector regression (SVR), a Gaussian process regression (GPR), random forests (RFs), decision trees (DTs), or artificial neural networks (ANNs). For example, Gaussian processes (GPs) are discussed in C. E. Rasmussen and C. K. I. Williams, “Gaussian Processes for Machine Learning,” the MIT Press, 2006, ISBN 026218253X (also available at www.GaussianProcess.org/gpml), which is incorporated by reference in its entirety.

The supervised model is not limited to considering functionality only, but can also account for other factors, such as similarity and stability, that can increase the likelihood of candidate amino acid sequences succeeding. Consider that, physics-based modeling, such as numerical computations using the Rosseta Commons software suite, can provide indicia of stability. For example, sequences that are predicted based on numerical computations to preserve the native structure of the natural fold are more likely to be functional.

Thus, the supervised model can be extended to account for other factors including stability and similarity. For example, a stability landscape can be defined in the latent space and a similarity landscape can be defined in the latent space. Then multi-dimensional analysis can be used to determine a Pareto frontier (i.e., a convex hull in the multi-dimensional space of (i) functionality, (ii) similarity, and (iii) stability), and the candidate amino acid sequences can be selected from points in the latent space lying on the Pareto frontier.

General Description of Method

FIG. 1B shows a more detailed flow diagram of method 10, according to one non-limiting embodiment.

In process 22, a training dataset 15 is used to train an amino acid sequence model. As a non-limiting example, a training dataset of N=1388 sequences in a multi-segment alignment (MSA) in which each sequence has a length L of 245 could be used as a training dataset 15.

In step 20 of process 22, a sequence model generates candidate amino acid sequences 25. As shown schematically in FIGS. 1C and 1D, the sequence model has a compress stage and a generate stage. The compress stage of the model can also be referred to as encoding (for example, encoding being a mapping from the amino acid sequence space to the latent space), and generation stage of the model can also be referred to as decoding (e.g., decoding being a mapping from the latent space to the amino acid sequence space).

After process 22, in step 30 of process 32, genes are synthesized to code for the candidate sequences 25, and candidate proteins 35 are produced from the synthesized genes. In some implementations, virtual screening can be used to speed up this search. Virtual libraries containing thousands to hundreds of millions of candidates can be assayed with first-principles simulations or statistical predictions based on learned proxy models, and only the most promising leads are selected and tested experimentally.

In step 40 of process 42, assays are performed to evaluate the degree to which the candidate proteins exhibit the desired functionality. Measured value 45 indicative of desired functionality obtained from these assays can be considered in step 50 to determine whether the stopping criteria have been satisfied. If the stopping criteria are reached, the optimized amino acid sequences are output as the designed proteins 55. If not, the method 10 continues to step 60.

In FIG. 1D, process 22 is illustrated as computational modeling and step 32 is illustrated as gene synthesis and protein production, which is followed by high-throughput functional screening. The measured values of the high-throughput functional screening are fed back into the computational model, unless the high-throughput functional screening indicates that the protein design objective has been met. In that case the method is complete, and method 10 outputs the designed proteins with the desired characteristics. In the example implementation of FIG. 1D, the high-throughput functional screening is illustrated using a micro-fluidics apparatus that measures the fluorescence of cells containing respective of the candidate proteins, and directing or providing the cells to different bins based on the measured fluorescence. It is to be understood, however, that alternative methods and systems (e.g., alternative or different from a micro-fluidics apparatus) may also be used to perform high-throughput functional screening to determine measured values as described herein. The measured fluorescence in this implementation is adjusted to be proportional to the protein properties that define the design objectives. Thus, the screen produces data for iterative optimization of the computational models.

In step 60 of process 42, a functionality model is generated based on the measured values.

Designed or Candidate Proteins

While the disclosure has illustrated the use of the method and system described herein to generate new enzymes, the instant system and method is not limited to proteins (e.g., such as the designed or candidate proteins described herein) having a particular type of activity or structure. Indeed, in various aspects, a protein or otherwise designed or candidate protein may be an antibody, an enzyme, a hormone, a cytokine, growth factor, clotting factor, anticoagulation factor, albumin, antigen, an adjuvant, a transcription factor, or a cellular receptor. Indeed, the systems and methods described herein are useful for, e.g., generating or identifying novel proteins which exhibit a biological activity consistent with an antibody, an enzyme, a hormone, a cytokine, growth factor, clotting factor, anticoagulation factor, albumin, antigen, an adjuvant, or a cellular receptor. In addition, the candidate proteins described herein may be used in various applications or functions. By way of example the candidate proteins may be used for selected binding to one or more other molecules. Additionally, or alternatively, as a further example, the candidate proteins may be provided to catalyze one or more chemical reactions. Additionally, or alternatively, as a further example, the candidate proteins may be provided for long-range signaling (e.g., allostery comprising a process by which biological macromolecules (mostly proteins) transmit the effect of binding at one site to another, often distal, functional site, allowing for regulation of activity).

Cytokines include, but are not limited to, chemokines, interferons, interleukins, lymphokines, and tumor necrosis factors. Cellular receptors, such as cytokine receptors, also are contemplated. Examples of cytokines and cellular receptors include, but are not limited to, tumor necrosis factor alpha and beta and their receptors; lipoproteins; colchicine; corticotrophin; vasopres sin; somatostatin; lypres sin; pancreozymin; leuprolide; alpha-1-antitrypsin; atrial natriuretic factor; thrombin; enkephalinase; RANTES (regulated on activation normally T-cell expressed and secreted); human macrophage inflammatory protein (MIP-1-alpha); cell determinant proteins such as CD-3, CD-4, CD-8, and CD-19; erythropoietin; interferon-alpha,-beta,-gamma,-lambda; colony stimulating factors (CSFs), e.g., M-CSF, GM-CSF, and G-CSF; IL-1 to IL-10; T-cell receptors; and prostaglandin.

Examples of hormones include, but are not limited to, anti-diuretic hormone (ADH), oxytocin, growth hormone (GH), prolactin, growth hormone-releasing hormone (GHRH), thyroid stimulating hormone (TSH), thyrotropin-release hormone (TRH), adrenocorticotropic hormone (ACTH), follicle-stimulating hormone (FSH), luteinizing hormone (LH), luteinizing hormone-releasing hormone (LHRH), thyroxine, calcitonin, parathyroid hormone, aldosterone, cortisol, epinephrine, glucagon, insulin, estrogen, progesterone, and testosterone.

Examples of growth factors include, e.g., vascular endothelial growth factor (VEGF), nerve growth factor (NGF), platelet-derived growth factor (PDGF), fibroblast growth factor (FGF), epidermal growth factor (EGF), transforming growth factor (TGF), bone morphogenic proteins (BMPs), and insulin-like growth factor-I and —II (IGF-I and IGF-II).

Examples of clotting factors or a coagulation factors include Factor I, Factor II, Factor III, Factor V, Factor VI, Factor VII, Factor VIII, Factor VIIIC, Factor IX, Factor X, Factor XI, Factor XII, Factor XIII, von Willebrand factor, prekallikrein, heparin cofactor II, antithrombin III, and fibronectin.

Examples of enzymes include, but are not limited to, angiotensin converting enzyme, streptokinase, L-asparaginase, and the like. Other examples of enzymes include, e.g., nitrate reductase (NADH), catalase, peroxidase, nitrogenase, phosphatase (e.g., acid/alkaline phosphatases), phosphodiesterase I, inorganic diphosphatase (pyrophosphatase), dehydrogenase, sulfatase, arylsulfatase, thiosulfate sulfurtransferase, L-asparaginase/L-glutaminase, beta-glucosidase, aryl acylamindase, amidase, invertase, xylanase, cellulose, urease, phytases, carbohydrase, amylase (alpha-amylase/beta-amylase), arabinoxylanase, beta-glucanase, alpha-galactosidase, beta-mannanase, pectinase, non-starch polysaccharide degrading enzymes, endoproteases, exoproteases, lipases, cellulases, oxidoreductases, ligases, synthetases (e.g., aminoacyl-transfer RNA synthetase; glycyl-tRNA synthetase), transferases, hydrolases, lyase (e.g., decarboxylases, dehydratases, deaminases, aldolases), isomerases (e.g., triose phosphate isomerase), and trypsin. Further examples of enzymes include catalases (e.g., alkali-resistant catalases), alkaline amylase, pectinase, oxidase, laccases, proxidases, xylanases, mannanases, acylases, alcalase, alkylsulfatase, cellulolytic enzymes, cellobio-hydrolase, cellobiase, exo-1,4-beta-D-glucosidase, chloroperoxidase, chitinase, cyanidase, cyanide hydratase, 1-Galactono-lactone oxidase, lignin peroxidase, lysozyme, mn-peroxidase, muramidase, parathion hydrolase, pectinesterase, peroxidase, and tryosinase. Further examples of enzymes include nuclease (e.g., endonuclease, such as zinc finger nucleases, transcription activator-like effector nuclease, Cas nucleases, engineered meganucleases).

The disclosure above references exemplary proteins within various protein classes and subclasses (e.g., progesterone as an example of a hormone). It will be appreciated that the system and method described herein may generate a protein which diverges from a particular protein listed above as far as amino acid sequence, but which has similar, equivalent, or improved activity or other desired biological feature exhibited by a reference protein.

Protein Production

Recombinant protein is achieved using a variety of biotechnology tools and methods. For example, an expression vector comprising a nucleic acid encoding a protein of interest is introduced into a host cell, which is cultured under conditions which allow for protein production, and the protein is collected by, e.g., purifying secreted protein from the culture media or lysing cells to release intracellular protein and collecting the protein of interest from the lysate.

Methods of introducing nucleic acids into host cells are well known in the art and described in, e.g., Cohen et al (1972) Proc. Natl. Acad. Sci. USA 69, 2110; Sambrook et al (2001) Molecular Cloning, A Laboratory Manual, 3.sup.rd Ed. Cold Spring Harbor Laboratory, Cold Spring Harbor, N.Y.; and Sherman et al (1986) Methods In Yeast Genetics, A Laboratory Manual, Cold Spring Harbor, N.Y.

Nucleic acid encoding a protein is typically packaged in an expression vector with regulatory sequences that promote protein production and, optionally, which facilitate entry into host cells. Plasmids or viral vectors are often utilized in this context.

A variety of host cells are suitable for recombinant protein production. Mammalian cells, for example, are often used to produce therapeutics. Non-limiting examples of mammalian host cells suitable for recombinant protein production include, but are not limited to, Chinese hamster ovary cells (CHO); monkey kidney CV1 cells transformed by SV40 (COS cells, COS-7, ATCC CRL-1651); human embryonic kidney cells (e.g., 293 cells); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells; human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); and mouse mammary tumor cells (MMT 060562, ATCC CCL-51). Bacterial cells (gram-positive or gram-negative) are prokaryotic host cells suitable for protein production. Yeast cells are also suitable for recombinant protein production.

End Products

FIG. 1E shows a diagram illustrating example designed or candidate proteins 1e10 (e.g., design proteins or candidate proteins 1e20, 1e22, 1e24, and/or 1e26) resulting from the systems and/or methods described herein, where such proteins may be provided (e.g., as end products) in one or more alternative forms and/or applied to various industries (e.g., industries 1e30, 1e32, 1e34, 1e36, and/or 1e38);

The proteins (e.g., candidate proteins) resulting from the systems and/or methods described herein may be applied to any of a number of industries, including biopharma (e.g., therapeutics and diagnostics) 1e34, agriculture (e.g., plants and livestock) 1e32, veterinary, industrial biotech (e.g., biocatalysts) 1e30, environmental conservation and remediation 1e38, and energy 1e36.

As illustrated by FIG. 1E, proteins (e.g., designed or candidate proteins) resulting from the systems and/or methods described herein may be developed or generated for or otherwise used in various industries (e.g., 1e30, 1e32, 1e34, 1e36, and/or 1e38) and provided to an end user in a number of one or more alternative forms: (1) as purified proteins 1e20 supplied either in solution, as lyophilized powder, or other forms commonly used for transmission of active protein molecules, (2) as libraries of synthetic genes 1e22 encoding the designed proteins cloned in plasmid, viral, or cosmid vectors, (3) as genes expressed in engineered microbial or other host strains 1e24, and/or (4) as genes cloned into gene therapy vectors 1e26. By way of non-limiting example, as illustrated by FIG. 1E, purified proteins 1e20 may be used in or provided to any one or more of the industries of biocataysis 1e30, agriculture 1e32, and/or biopharma 1e34 for the development, manufacture, or creation of end products. As an additional non-limiting example, libraries of synthetic genes 1e22 may be used in or provided to any one or more of the industries of biocataysis 1e30, agriculture 1e32, biopharma 1e34, and/or environmental conservation and remediation 1e38 for the development, manufacture, or creation of end products. As an additional non-limiting example, engineered microbial or other host strains 1e24 may be used in or provided to any one or more of the industries of biocataysis 1e30, agriculture 1e32, biopharma 1e34, energy 1e36, and/or environmental conservation and remediation 1e38 for the development, manufacture, or creation of end products. As an additional non-limiting example, gene therapy vectors 1e26 may be used in or provided to any one or more of the industries of agriculture 1e32, and/or biopharma 1e34 for the development, manufacture, or creation of end products. Sections below herein provide additional details, and non-limiting examples, about these different approaches for generation, manufacture, delivery, use, and/or otherwise output of engineered protein solutions (e.g., designed or candidate proteins) for the development, manufacture, creation, and/or generation of end products as described herein.

Purified Proteins

The output of the methods and/or systems described herein may be provided to an end user as a purified protein product (e.g., purified proteins 1e20). Proteins may be expressed and purified in any of a number of ways. Protein expression often involves, but is not limited to, introduction (transformation) of genes encoding the protein of interest into host microorganisms, growth of transformed strains to exponential phase, and induction of gene expression using small molecules that activate transcription from one of many standard promoter sequences. Induced cultures are then grown to saturation phase and harvested for protein purification. Examples of protein purification techniques include, but are not limited to, centrifugation, filtration (e.g., tangential flow microfiltration), precipitation/flocculation, chromatography (e.g., ion exchange chromatography, immobilized metal chelate chromatography, and/or hydrophobic interaction chromatography), thiophilic adsorption, affinity-based purification methods, crystallization, and the like. Purified proteins may be formulated into liquid compositions or lyophilized (or dried) compositions suitable for shipment, storage, and an ultimate end use in, e.g., therapeutic, agricultural, and/or industrial fields.

Gene Libraries

The output of the methods and/or systems described herein may be provided as genes or gene libraries (e.g., genes or gene libraries 1e22). Genes encoding the designed proteins may be created through back translation of the amino acid sequences into DNA sequences using standard codon tables that may then be cloned into one of various host plasmid or viral vectors for propagation and amplification. The resulting libraries may then be used by the end user in various custom manufacturing processes in pharmaceuticals, agricultural products, biocatalysis, and environmental remediation. It is standard to prepare gene libraries as pure DNA supplied in a dried (or lyophilized) form for storage and/or shipment.

Engineered Microorganisms

The output of the methods and/or systems described herein may be provided as genes integrated into the chromosome of host organisms through standard transgenesis methods. In this case, genes encoding the designed proteins may be created through back translation of the amino acid sequence into nucleic acid sequences using standard codon tables, ligated with appropriate 5′ and 3′ nucleotide sequences to ensure desired expression, regulation, and mRNA stability, and integrated into the host organism genome. Such engineered host organism (e.g., engineered strains 1e24) may be supplied to end users for use in one of many standard applications. This could include growth in large scale culturing and production facilities, use in multistep industrial biosynthetic pathways, or as a component of engineered microbial communities for industrial, agricultural, pharmaceutical, environmental, or energy harvesting processes.

Gene Therapy Vectors

In various embodiments, the output of the method or system described herein is a nucleic acid with a desired activity (e.g., with a desired activity itself or which encodes a peptide or protein with a desired activity). In various aspects, the nucleic acid is incorporated into an expression vector (e.g., vector 1e26). A “vector” or “expression vector” is any type of genetic construct comprising a nucleic acid (DNA or RNA) for introduction into a host cell. In various embodiments, the expression vector is a viral vector, i.e., a virus particle comprising all or part of the viral genome, which can function as a nucleic acid delivery vehicle. Viral vectors comprising exogenous nucleic acid(s) encoding a gene product of interest also are referred to as recombinant viral vectors. As would be understood in the art, in some contexts, the term “viral vector” (and similar terms) may be used to refer to the vector genome in the absence of the viral capsid. Viral vectors for use in the context of the disclosure include, for example, retroviral vectors, herpes simplex virus (HSV)-based vectors, parvovirus-based vectors, e.g., adeno-associated virus (AAV)-based vectors, AAV-adenoviral chimeric vectors, and adenovirus-based vectors. Any of these viral vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989); Ausubel et al., Current Protocols in Molecular Biology, Greene Publishing Associates and John Wiley & Sons, New York, N.Y. (1994); Coen D. M, Molecular Genetics of Animal Viruses in Virology, 2nd Edition, B. N. Fields (editor), Raven Press, N.Y. (1990) and the references cited therein.

Expression vectors have a variety of uses in industry. For example, the expression vector may be provided to an end user for, e.g., use in gene therapy methods (i.e., involving administration to a patient to treat or prevent a disease or disorder). The expression vector may be used by an end user to produce proteins of interest encoded by the nucleic acid.

Gaussian Process Regression (GPR)

One non-limiting example for generating the functionality model applies Gaussian process regression (GPR) to measured values from the assays. For example, GPR can be performed on the measured values, as discussed in P. A. Romero, et al., “Navigating the protein fitness landscape with GPs,” PNAS, vol. 110, pp. E193-E201 (2012); C. N. Bedbrook et al., “Machine learning to design integral membrane channel rhodopsins for efficient eukaryotic expression and plasma membrane localization.” PLoS Comput Biol., vol. 13, p. e1005786 (2017); and R. G. Bombarelli, et al., “Automatic Chemical Design Using a Data-Driven Continuous Representation of Molecules,” ACS Cent. Sci., vol. 4, pp. 268-276 (2018), each of which is incorporated herein by reference in its entirety. GPR is performed on the measured values with respect to the positions in latent space corresponding to the respective candidate proteins, resulting in each position in the latent space being assigned a mean value and a standard variation (i.e., uncertainty).

The mean value and standard variation landscapes generated by GPR can be used to select candidate positions in the latent space based on different but complementary objectives. On the one hand, the objective when in an exploitation mode is to select the candidate positions that are most likely to function optimally (e.g., regions on the latent space having the largest mean value). On the other hand, the objective when in an exploration mode is to select candidate positions that most improve the functionality model. These positions can be regions in the latent space having the largest uncertainties because more samples in these regions can narrow the uncertainty the most and improve the predictive power of the functionality model. The objectives of the exploration and exploration modes are coupled because improving functionality model can result in better predictions regarding which points in the latent space corresponding to optimal functionality. Further, a large number of candidates are selected during each iteration, and therefore both exploration and exploration objectives can be targeted through selecting a subset of points based each of the respective objectives or based on a combination thereof.

The mean value as a function of position can define the functionality landscape. As described in more detail below, when in an exploitation mode, the functionality landscape can be used to select those candidate sequences from those regions within the landscape corresponding to peaks in the desired functionality. Further, when in an exploration mode, regions with large uncertainty can be identified for exploration by selecting candidate proteins in these regions to better estimate the degree of desired functionality exhibited in these regions. Both exploitation and exploration can be pursued simultaneously by selecting some of the candidate proteins according to the exploitation criterion and others of the candidate proteins according to the exploration criterion.

Further, in step 20 the candidate sequences 25 from the previous iteration can be used to update and refine the sequence model. For example, the candidate sequences 25 from the previous iteration can be added to the training data, and the sequence model can be further trained using the expanded/updated training dataset. Updating the sequence model can shift or distort the latent space. Therefore, the sequence model can be updated before calculating the functionality model and the fitness function 65. In certain implementations, the fitness function 65 is the functionality landscape. In other implementations, the fitness function 65 includes the functionality landscape combined with other indicia predicting which amino acid sequences are promising (e.g., stability).

In certain implementations, fitness landscapes are constructed to map the peaks and valleys of fitness over the protein sequence space. For example, the descriptors (inputs) are protein sequences and the readouts of fitness (outputs) are measures of desirable characteristics (i.e., fitness). In certain implementations, the outputs can be multi-dimensional measures of desirable characteristics. These measures of desirable characteristics can include, but are not limited to, activity, similarity to existing natural sequences, and protein stability. When the fitness of a protein sequence is multi-dimensional and contains multiple attributes, a separate landscape can be constructed for each component of the fitness.

The training data for determining the fitness will come from experimental assays over the sequences produced via gene synthesis and expression. For example, experimental assays can measure functional activities including but not limited to binding, catalytic activity, stability, or surrogates thereof (e.g., growth rate under environmental conditions that make it proportion to one or more of the functional activities). Further, in some implementations, computational modeling can also provide feedback that is used to determine fitness, e.g., computational modeling of the sequences can be used to predict protein stability.

Using the reduced-dimensional latent space for the domain of inputs to the fitness function can be more effective than using the sequence space for the domain. This is because the size of sequence space and the complexity of amino acid interactions make the construction of supervised regression models taking as input the protein sequence directly rather ill-posed. Accordingly, it is preferred to perform the regression by taking as inputs the representation of each sequence projected into the low-dimensional latent space.

The supervised learning model can be fit using, but not limited to, multivariable linear regression, nonlinear regression, support vector regression (SVR), Gaussian process regression (GPR), random forests (RFs), and artificial neural networks (ANNs).

In certain implementations, the candidate sequences will be generated using a two tier approach. In the first tier, the sequence based model (e.g., SCA, DCA, VAE, etc.) selects a first set of sequences, and then, in the second tier, the fitness model selects, as the candidate sequences, a subset of the first set of sequence based on fitness criteria (i.e., sequences corresponding to points near the peaks of the fitness landscape). That is, the first set of sequence are valuated computationally according to the fitness landscapes defining each component of fitness, and out of the first set of sequence generated sequences, one or more are identified as the top candidates to pass forward to be experimentally synthesized and assayed.

Multi-objective/Multi-dimensional Optimization in Latent Space

In implementations performing multi-objective optimization, the top sequences are identified as those that perform well along all components of fitness defined by the various landscapes discussed above (e.g., similarity and stability). Since the components of fitness may serve as conflicting objectives (e.g., activity may be inversely correlated with stability) so there is no single optimum solution (i.e., single best sequence), the best sequences can be identified using a Pareto frontier to solve the multi-objective optimization problem.

That is, multi-objective optimization problem is solved by narrowing the possible choices of best sequence to a lower-dimensional surface in a multi-dimensional space. In other words, the best sequences will lie on the Pareto frontier (also referred to as an optimal frontier or an efficient frontier). This frontier and the sequences lying on it can be identified using approaches selected from, but not limited to, scalarization, sandwich algorithms, normal boundary intersection (NBI), modified NBI (NBIm), normal constraint (NC), successive Pareto optimization (SPO), directed search domain (DSD), non-dominated sorting genetic algorithm-II (NSGA-II), strength Pareto evolutionary algorithm 2 (SPEA-2), particle swarm optimization, and simulated annealing.

For example, one way to perform multi-objective optimization problem is to simultaneously maximize all of the competing constituents of fitness through determination of the Pareto frontier. This is inherently an exploitation search because it focuses on finding candidates that have high fitness. In this implementation, the fitness model passes through those of the first sequences that the regression models predict as having a high degree of fitness and blocks those of the first sequences that are not predicted as having a high degree of fitness.

However, if a high degree of fitness is the only criteria for selection by the functionality/fitness model, then under-sampled regions of the search space might go unexplored because the high uncertainty in these under-sampled regions can preclude predictions of a high degree of fitness. Accordingly, the functionality/fitness model can also select candidates according to an exploration criterion in which the multi-objective optimization is solved not to maximize each component of fitness defined by the various regression landscapes, but is instead solved to identify those sequences that have the highest uncertainty in the fitness prediction models. In other words, the goal of the exploration criterion is to identify sequences where the regression models have the most uncertainty. Based on the exploration criterion, the sequences form the first set of sequences that correspond to regions having the most uncertainty are passed through as candidate sequences for experimental synthesis and assay. This exploration criterion is desirable because, when the regression models are highly uncertain about the properties of these sequences, collecting experimental data for these sequences does the most to improve the models (i.e., decrease their uncertainty) Therefore, exploration is important for providing additional training data to retrain the model and strengthen its predictive performance.

In general, the protocol of judiciously selecting sequence based on these exploit/explore criteria is known as active learning. That is, a machine learning model is directing the collection of new experimental data to (i) guide experiment towards the most promising candidates, and (ii) direct the collection of new data most valuable for model retraining to improve its predictive performance. In this way, the model building and experimental synthesis operate within a positive-feedback cycle.

In certain implementations of method 10, the active learning problem is addressed using the multi-objective optimization techniques described above in combination with Bayesian optimization techniques to control the explore-exploit trade-off employing acquisition functions including, but not limited to, probability of improvement, expected improvement, lower/upper confidence bound.

Multi-iteration Optimization

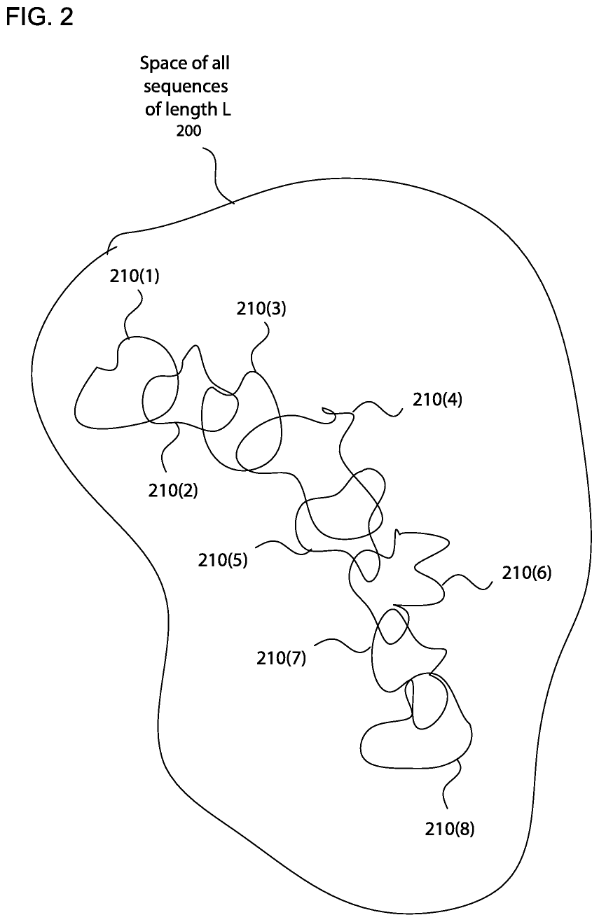

FIG. 2 illustrates a possible pathway traversed over eight iterations of method 10. Region 210(1) represents a locus of points within the set 200 of all possible sequences of length L. In practice the set 200 can be many of orders of magnitude larger than the subset 210(i) for the candidate sequences in the i-th iteration of method 10. Accordingly, FIG. 2 is not to scale. The subset 210(2) of candidate sequences generated in the second iteration can be shifted relative to the subset 210(1) of the first iteration, and each subsequent subset 210(i+1) can be shifted relative to the previous subset 210(i). Thus, as the iteration number i increases, the subset 210(i) of the candidate sequence explores more the space 200 and evolves towards a peak in the functionality landscape, until the sought after level of the desired functionality is satisfied.

Further, method 10 can be used to achieve a desired functionality within a new environment. For example, a known enzyme might exist that has desired catalytic function at temperature X, but a new enzyme is desired that has the same catalytic function at temperature Y. To design this new enzyme, a series of intermediate temperatures X<A<B<C<D<Y can be selected. Then starting from the known enzyme and its homologs, a first set of enzymes can be designed using method 10 in which assays are performed at temperature A to measure the desired catalytic function. Then, the method 10 can be repeated starting with the first set of enzymes, but this time maintaining the assays at temperature B to generate a second set of enzymes that exhibits the desired catalytic function when in an environment at temperature B. This is repeated a third, fourth and fifth time at temperatures C, D, and Y respectively, until the final set of enzymes exhibits the desired catalytic function when at temperature Y. Thus, method 10 can be used to achieve a particular functionality in a new environment (e.g., at different temperature, pressure, light conditions, pH, or different chemical/elemental concentrations in the solution/environment).

Recent advances in machine learning have resulted in powerful probabilistic generative models that, after being trained on real examples, are able to produce realistic synthetic samples. Such models usually also produce low-dimensional continuous representations of the data being modeled, allowing interpolation or analogical reasoning. As discussed above these generative models are applicable to protein design, e.g., using a pair of deep networks trained as an auto-encoder to convert proteins represented as sequences of amino acids into a continuous vector representation.

Supervised and Unsupervised Learning

As described in various embodiments herein, a machine learning model may be trained using a supervised or unsupervised machine learning program or algorithm. The machine learning program or algorithm may employ a neural network, which may be a convolutional neural network, a deep learning neural network, or a combined learning module or program that learns in two or more features or feature datasets in particular areas of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve B ayes analysis, clustering, reinforcement learning, and/or other machine learning algorithms and/or techniques. Machine learning may involve identifying and recognizing patterns in existing data (such as candidate proteins in training dataset(s) of amino acid sequences of proteins) in order to facilitate making predictions, classifications, or outputs for subsequent data (e.g., synthesizing candidate genes and producing candidate proteins corresponding to the respective candidate amino acid sequences).

Machine learning model(s), such as those described herein, may be created and trained based upon example (e.g., “training data,”) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, classify, or output, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated. The disclosures herein may use one or both of such supervised or unsupervised machine learning techniques.

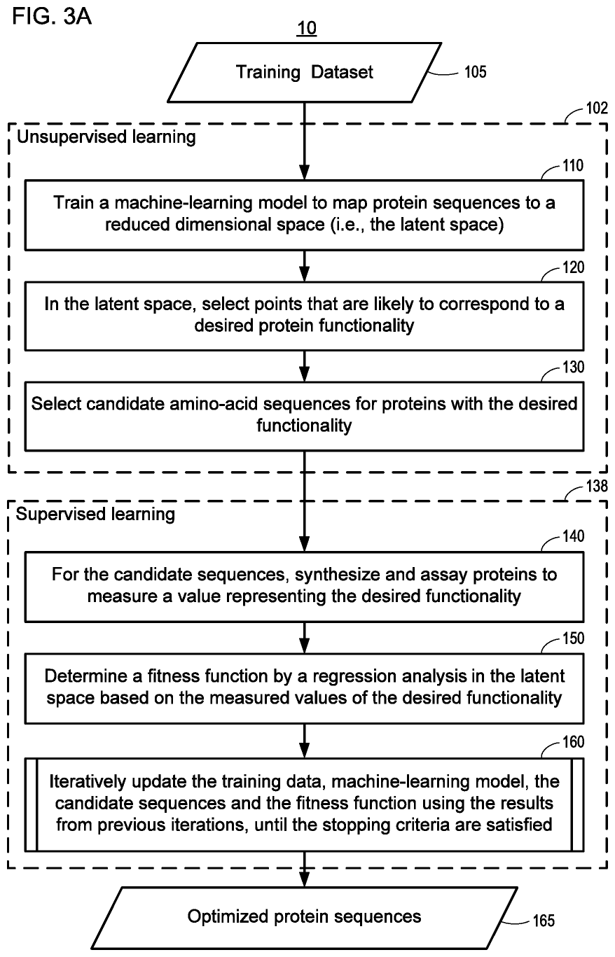

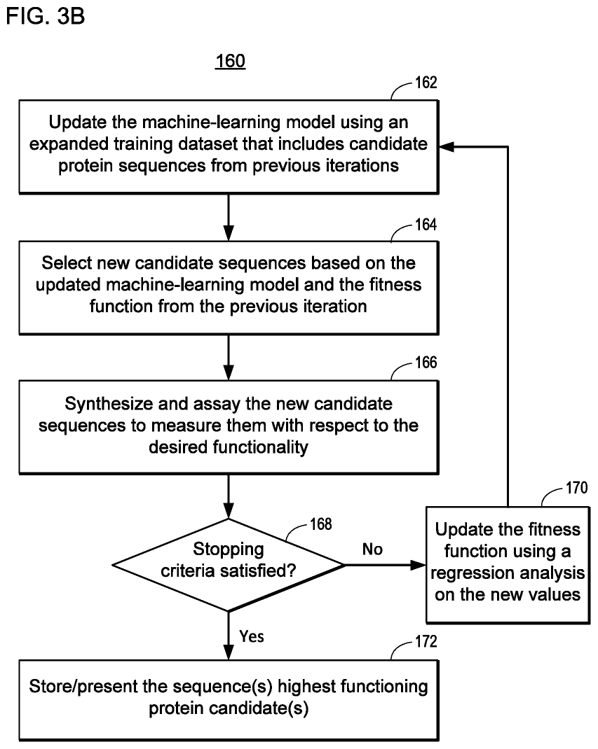

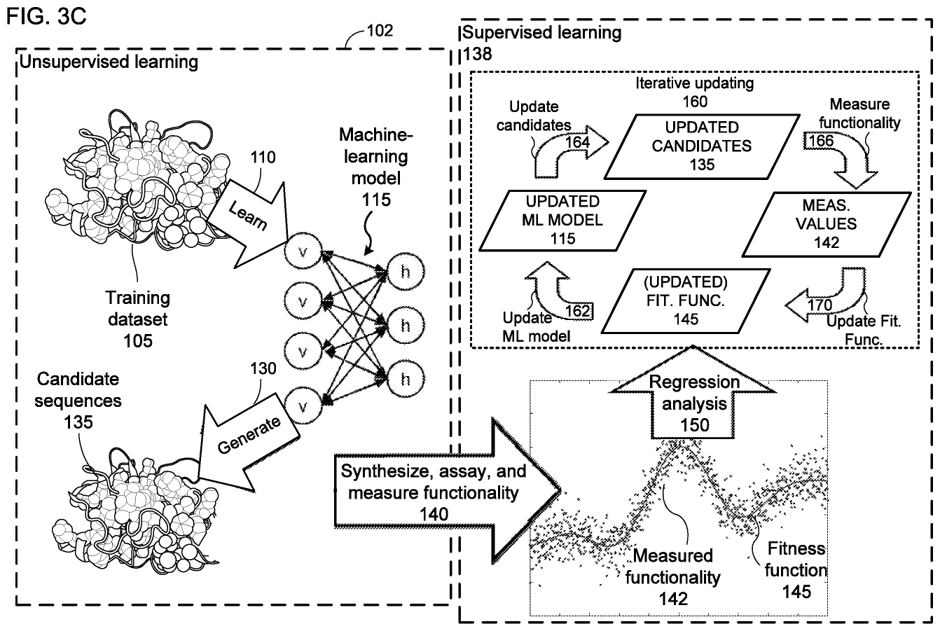

FIGS. 3A, 3B, and 3C illustrate another non-limiting implementation of a method 10. For example, FIGS. 3A, 3B, and 3C are illustrated using the non-limiting example of the sequence model (i.e., the unsupervised model) is a VAE or an RBM. Method 10 can be subdivided into two parts: (i) an unsupervised learning process 102, and (ii) a supervised learning process 138. The unsupervised learning process 102 starts with a training dataset 105 and generates therefrom candidate sequences 135 that are likely to exhibit given quantitative desiderata (e.g., have a desired functionality). In the unsupervised learning process 102, a machine learning model 115 is trained to map back and forth between visible variables (i.e., the amino acid sequence of the proteins) and hidden variables, which define a latent space. The machine learning model 115 can be a generative model. The latent space has a reduced dimension relative to the dimension of the amino acid sequence (e.g., an amino acid sequence of length N can have a dimension 20^{N} to account for the 20 possible amino acids at each of the N residues). To map from a higher dimensional space to a lower dimensional space, machine learning model 115 learns implicit patterns and correlations among the residues of the respective amino acid sequences in the training data set to encode the information more compactly. That is, the mapping from the visible variables to the hidden variables correlations compresses the information to more compactly represent this information using reduced dimensions. Thus, patterns and correlations among the amino acid sequences in the training dataset are learned and expressed implicitly in the machine learning model 115.

Additionally, the machine learning model 115 can be used to generate new amino acid sequences. A set of proteins having similar functionality can define a cluster of points when mapped to the latent space. For example, the mean and variances of this cluster can be used to define a multivariate Gaussian distribution to represent a probability density function (pdf) of the cluster. By randomly selecting points within this and then using the machine learning model 115 to map these points back to amino acid sequences, the machine learning model 115 can generate candidate sequences 135 for new synthetic proteins that are likely to have similar structure and therefore similar functionality to the original proteins that were used to define the cluster.

The supervised learning process 138 starts with the candidate sequences 135, and synthesizes genetic sequences to produce candidate proteins, which have the candidate sequences 135. The properties of these candidate proteins are then evaluated. For example, the candidate proteins can be assayed to measure the degree to which they exhibit the desired functionality (e.g., the desiderata). A fitness function 145 is then determined from the measured values 142, and an iterative cycle is begun to search for better proteins by updating the machine learning model 115, the candidate sequences 135, the measured values 142, and the fitness function 145 in an iterative manner until some predefined stopping criteria are reached.

Regarding the desired functionality, proteins display the following properties, which in various combinations, could contribute to a desired design objective: (i) folding rate and yield (the speed of folding and the probability of folding), (ii) thermodynamic stability (the difference in free energy between the unfolded and folded states), (iii) binding affinity (the free energy separating the unbound and bound states), (iv) binding specificity (the difference in binding between desired and not desired substrates), (ν) catalytic power (the free energy separating the enzyme-substrate complex and the transition state for a reaction), (vi) allostery (the long-range communication of amino acids in a protein), and (vii) evolvability (the capacity to generate heritable variation).

As discussed above high-throughput assays can be tailored to generate measured values for evaluating the desired functionality. Here various examples of types of assays corresponding to respective properties of proteins are provided. Folding rate and yield can be evaluated, e.g., by an assay directed to following compactness by gel filtration chromatography or fluorescence of environmentally sensitive fluorophores. Thermodynamic stability can be measured, e.g., using differential scanning calorimetry or 1H-15N HSQC NMR. Binding can be measured by fluorescence or titration calorimetry methods (e.g., using droplet microfluidics) or two-hybrid gene-expression methods in cells. Catalytic power can be measured by absorbance/fluorescence methods (e.g., using droplet microfluidics) or by in survival or growth rate measurements. Allostery can be measured in various methods among which is measuring allostery via regulation in cells. Evolvability can be measured through comparing the sensitivity of amino acids to deep mutational scans in the context of both current and novel functions.

In certain implementations, the approach used for method 10 can be broken down into two parts (i) unsupervised learning of a low-dimensional latent-space embedding of protein sequences, and (ii) supervised learning of a functionality landscape within the latent space. That is, method 10 performs a search to find low-dimensional representations of protein sequences that preserve the key information about their sequence and function and predict from these representations the likely functionality of new sequences with even better performance.

In step 110 of method 10, the machine learning model 115 is trained using the training dataset 105. In one non-limiting example, the machine learning model 115 is a variational auto encoder (VAE). In another non-limiting example, the machine learning model 115 is a restricted Boltzmann machine (RBM). Both VAEs and RBMs are generative models, and other generative models can be used for the machine learning model 115, as would be understood by a person of ordinary skill in the art. As discussed above, in addition to VAEs and RBMs, the unsupervised learning can be performed using generative-adversarial networks (GANs), statistical coupling analysis (SCA), or direct coupling analysis (DCA). The decision of which unsupervised learning methods to use used can be based on an empirical evaluation of which method offers the best performance in providing robust latent space representations and accurate generative performance.

Statistical Coupling Analysis (SCA) Model

In one non-limiting implementation, a statistical coupling analysis (SCA) model can be used as the sequence-based model, the training data is used to compute a SCA model, which is defined by a conservation-weighted correlation matrix (e.g., the SCA matrix of coevolution between all pairs of amino acids), as described in K. A. Reynolds, et al., “Evolution-Based Design of Proteins,” Methods in Enzymology, vol. 523, pp. 213-235 (2013) and discussed in O. Rivoire, et al., “Evolution-Based Functional Decomposition of Proteins,” PLoS Comput Biol, vol. 12, p. e1004817 (2016), each of which is incorporated herein by reference in its entirety. That is, the information of the training dataset is compressed by the SCA model into a single pairwise correlation matrix. Furthermore, a singular value decomposition or eigenvalue decomposition of the SCA-matrix reveals that most of the modes are indistinguishable from sampling noise, while the top few modes (corresponding to the latent space) capture statistically significant correlations. These top few modes define sectors, which comprise one or more groups of collectively evolving amino acids. SCA-based protein design can be performed by computational simulations that start with random sequences and evolve (in silico) synthetic sequences that are constrained by the observed evolutionary statistics as captured in the SCA coevolution matrix.

For example, SCA-based protein design uses a Metropolis Monte Carlo simulated annealing (MCSA) algorithm to explore the sequence space consistent with a set of applied constraints between amino acids. The MCSA algorithm is an iterative numerical method for searching for the global minimum energy configuration of a system starting from any arbitrary state and is especially useful when the number of possible states is very large and the energy landscape is rugged and characterized by many local minima. The energy function (or “objective function”) to be minimized can, in general, depend on many parameters of the system and represents the constraints that define the size and shape of the final solution space. In essence, the objective function can be thought of as the hypothesis being tested—the set of applied constraints that is used to test for specifying folding, thermodynamic stability, function, and any other aspects of protein fitness.

For SCA-based protein design, the system under consideration is a MSA (rather than a single sequence), and the objective function (E) is the summed difference between the correlation for a MSA of protein sequences during iterations of the design process and the target correlation matrix deduced from the natural MSA, e.g., as provided by:

$$E = {\sum }_{ijab} \vert {\tilde{C}}_{ij ( design )}^{( a b )} - {\tilde{C}}_{ij ( natural )}^{( a b )} \vert$$

With respect to the above formula, the weighted correlation tensors are given by, e.g.

\tilde{C}ᵢⱼ^{(ab)}=ϕᵢ^{(a)}ϕⱼ^{(b)}Cᵢⱼ^{(ab)}

In the above formula, Cᵢⱼ^{(ab)} represents the raw frequency-based correlations between each pair of amino acids (a, b) at each pair of positions (i,j), which is given by Cᵢⱼ^{(ab)}=ƒᵢⱼ^{(ab)}−ƒᵢ^{(a)}ƒᵢ^{(b)},ƒᵢ^{(a)} is the frequency of amino acid a at position i, and ƒᵢⱼ^{(ab)} is the frequency of a pair of amino acids (a, b) at positions (i,j), respectively, and ϕ represents a conservation-based weighting function. The lowest energy configuration for the designed MSA is the set of sequences that gives a pattern of correlations in the designed sequences (\tilde{C}_{ij(design)}^{(ab)}) that most closely reproduces that of the natural MSA (\tilde{C}ᵢⱼ₍ₙₐₜᵤᵣₐₗ₎^{(ab)}). At the limit of large numbers of sequences, this result is tantamount to drawing sequences from a maximum entropy probability distribution consistent with the applied set of observed correlations the gradient of relative entropy. Often the number of designed sequences can be much larger than can be measured even with high-throughput functional screening. For example, on the order of 1,000 candidate sequences might be reasonable to measure using high-throughput functional screening, but the total number of designed sequences (\tilde{C}_{ij(design)}^{(ab)})might be many orders of magnitude greater. For example, it is to be understood that the measured candidate sequences can comprise various numbers and counts including by way of non-limiting example, at least 100 candidate sequences, at least 500 candidate sequences, at least 1000 candidate sequences, at least 1500 candidate sequences, etc. Thus, the candidate sequences 25 can be randomly selected for the total number of designed sequences (\tilde{C}_{ij(design)}^{(ab)}).

Variational Auto-Encoder (VAE) Model

An example of using a VAE as the machine learning model 115 is now provided, and other embodiments are provided later in which other types of machine learning are used as the machine learning model 115.

In one non-limiting example, the machine learning model 115 can be a VAE employing three encoding layers and three decoding layers, which employ layer-wise batch normalization and dropout, Tan h activation functions, and a Softmax output layer. The VAE neural network can be trained on using a training dataset that includes on the order of 1,000 protein sequences under a one-hot amino acid encoding employing a loss function that is the sum of a binary cross entropy and Kullback-Leibler divergence and employ the Adam optimizer during training. For example, it is to be understood that the protein sequences in the training dataset can comprise various numbers and counts including by way of non-limiting example, at least 100 protein sequences, at least 500 protein sequences, at least 1000 protein sequences, at least 1500 protein sequences, etc. Training is terminated by early stopping when the loss function on a holdout validation partition no longer decreases in order to guard against overfitting. The number of VAEs is optimized over different train/test splits. Further, the dimensionality of the latent space is optimized to select the size of the latent space at which the validation loss no longer decreases with increased dimensionality. The optimum VAE is validated through its reconstruction accuracy on a test partition that was not used in training.

The trained model can be used to efficiently generate millions of new sequences by sampling the latent space using Gaussian random numbers and passing these through the decoder to turn them into protein sequences. Since experiments show that the latent space encodes nature's rules for viable proteins, the expectation is that samples from this latent space also produce viable proteins, some of which have not been produced by natural selection.

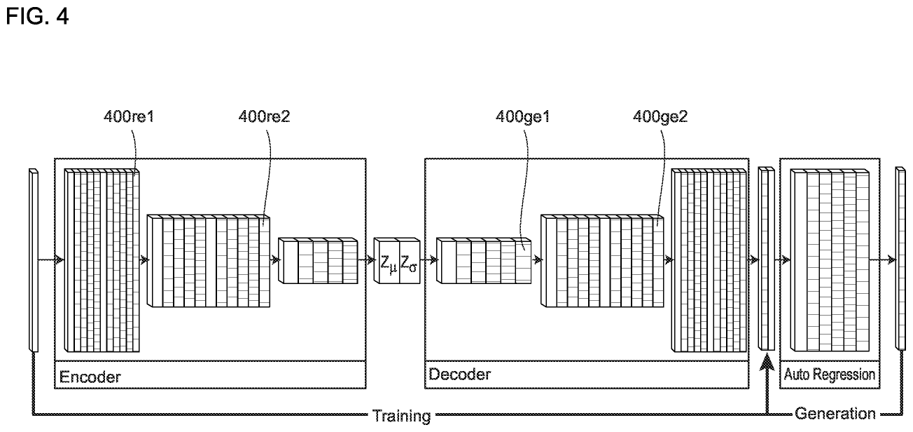

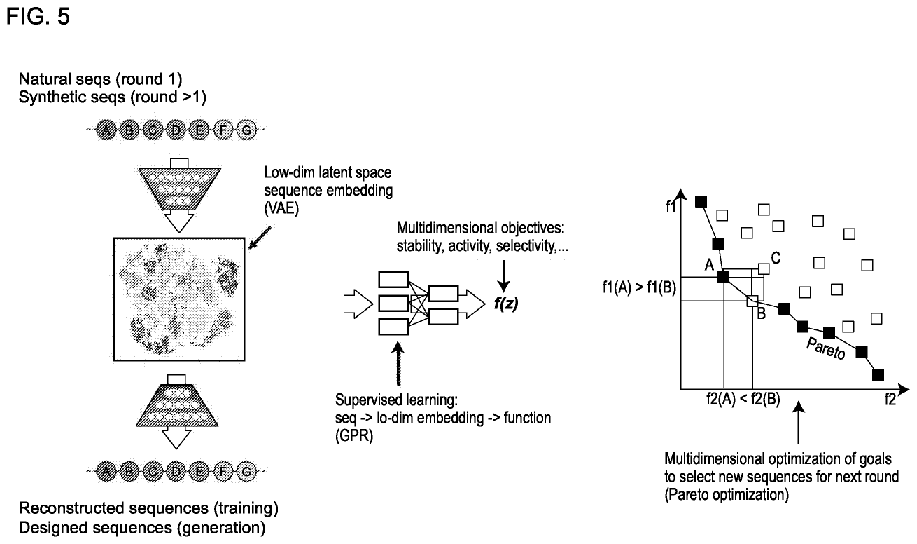

FIGS. 4 and 5 show schematic diagrams of VAEs that are used as generative models. In FIG. 4, the VAE includes an encoder section, a decoder section, and an auto regression section, as discussed in Costello, Z. and Garcia Martin, H., “How to hallucinate functional proteins,” Preprint at https://arxiv.org/abs/1903 (2019), which is incorporated herein by reference in its entirety. Inside of each module the shaded cubes each represent a layer type. Shaded layers indicate a 1D dilated convolutional layer with skip connections in the style of a residual network (resnet). The darkness of the shaded layers indicates the magnitude of the dilation. Progressively darker shades, or otherwise cross hatching, indicates larger dilation. This is done in the pattern used in FIG. 4. End layers (e.g., end layers 400re1 and 400re2) indicate a 1D convolution where the length of the input is halved with a stride of two and the channels are doubled. Transposed end layers (e.g., end layers 400ge1 and 400ge2) indicate the reverse operation of end layers (e.g., end layers 400re1 and 400re2) via a transposed one-dimensional (1D) strided convolution.

Generative models, such as the VAE, produce data with the same statistical properties that they were trained on. That is, when the VAE is trained on functional protein sequences that fold in their native host, the VAE should produce proteins that are likely to fold and function similarly to those in the training dataset. Thus, it can be verified that the model is behaving in a way consistent with this assumption.

The VAE is trained to reconstruct its own input. For example, the VAE first encodes a protein sequence into a feature vector (i.e., a vector in the latent space). The feature vector can be thought of as a summary of the important information in the protein sequence. The vector space where these feature vectors reside is generally known as the latent space. Then from that feature vector the variational auto-encoder reconstructs the original protein sequence. The loss function can represent the degree to which the output from the network matches the input. If the VAE were lossless, the output would always match the input. In general however, there is some loss in the VAE.

A fully trained VAE can be used in two modes: either as an encoder or as a decoder. The encoder can be used to take a protein sequence and find its associated feature vector. This feature vector can then be used for downstream classification or regression tasks. For example, it can be determined where a given protein is likely to be localized given its sequence. The decoder can be used to generate arbitrary sequences that are likely to fold and function by sampling from the latent space. Further the latent space samples can be chosen so that sequences are likely to also have desired phenotypes. In the remainder of the section the model design and its uses are described detail.

As shown in FIG. 5, the different groups of proteins (e.g., “A,” “B,” “C,” “D,” “E,” “F,” and “G”) localize and cluster to different regions within the latent space. The latent space also has the advantages of being a continuous space in contrast to the discretized space for the amino acid sequences. A protein representation method that is continuous and data-driven has several advantages, as discussed in R. Gomez-Bombarelli. et al., “Automatic Chemical Design Using a Data-Driven Continuous Representation of Molecules,” ACS Cent. Sci. Vol. 4, pp. 268-276 (2018) and S. Sinai et al., “Variational auto-encoding of protein sequences,” Preprint at https://arxiv.org/abs/1712.03346 (2018), which are both incorporated herein in its entirety, which is incorporated herein in by reference in their entirety. First, hand-specified mutation rules are unnecessary, as new compounds can be generated automatically by modifying the vector representation and then decoding. Second, using a differentiable model that maps from protein representations to desirable properties, it becomes possible to use a gradient-based optimization to make larger steps when searching the protein space. Gradient-based optimization can be combined with Bayesian inference methods to select amino acid sequences that are likely to be informative about the global optimum. Third, a data-driven representation can leverage large set of proteins (e.g., including proteins that exhibit the desired functionality as well as proteins that do not exhibit the desired functionality) to automatically build a larger implicit library, and then use the smaller set of the proteins that exhibit the desired functionality to build a regression model, which is incorporated into the fitness function 145) from the continuous representation to the desired properties. Thus, large protein databases can be used to train the VAE, even when many of the proteins have unknown properties.

Restricted Boltzmann Machine (RBM) Model

A non-limiting example of a machine learning model to perform unsupervised learning is an RBM (shown schematically in FIG. 3C). In the encode direction the amino acid sequences are applied as an input to a visible layer of neuronal nodes, and the hidden layer is calculated by taking a biased sigmoidal function of weighted sum of the values input to the visible layer. RBMs are a variant of Boltzmann machines, with the restriction that their neurons must form a bipartite graph: a pair of nodes from each of the two groups of units (commonly referred to as the visible and hidden units respectively) may have a symmetric connection between them; and there are no connections between nodes within a group. This restriction allows for more efficient training algorithms than are available for the general class of Boltzmann machines, in particular the gradient-based contrastive divergence algorithm. Restricted Boltzmann machines can also be used in deep learning networks as deep belief networks by stacking RBMs and optionally fine-tuning the resulting deep network with gradient descent and backpropagation.

Restricted Boltzmann machines are trained to maximize the product of probabilities assigned to some training set V (a matrix, each row of which is treated as a visible vector ν), as provided by:

$${arg max}_{w} {\prod }_{v ∈ V} P ( v )$$

Alternatively, and equivalently, to maximize the expected log probability of a training sample ν, the following formula may be used, e.g.:

$${arg max}_{w} 𝔼 log ( P ( v ) )$$

In the above formula(s), P(ν)=Σₕ P(ν, h)=Z⁻¹ Σₕ exp(−E(ν, h)) is the marginal probability of a visible vector of Booleans summed over all possible hidden layer configurations, where Z is a partition function, which normalizes the probability distribution P(ν, h), and the energy function is E(ν, h)=—α^{T} ν−−b^{T} h −ν^{T}Wh, wherein W is a matrix of weights associated with the connections between values of the hidden units h and visible units ν, and α are the bias weights (offsets) for the visible units with b being the bias weights (offsets) for the hidden units.

The RBM can be trained to optimize the weights W between the nodes using a contrastive divergence (CD). That is, Gibbs sampling is used inside a gradient descent procedure (similar to the way backpropagation is used inside such a procedure when training feedforward neural nets) to compute weight updates. In the single-step contrastive divergence procedure the following steps are performed: (i) take a training sample ν, compute the probabilities of the hidden units and sample a hidden activation vector h from this probability distribution; (ii) compute the outer product of ν and h and call this the positive gradient; (iii) from h, sample a reconstruction ν′ of the visible units, then resample the hidden activations h′ from this. (Gibbs sampling step); (iv) compute the outer product of ν′ and h′ and call this the negative gradient; (ν) let the update to the weight matrix W be the positive gradient minus the negative gradient, times some learning rate, e.g., as provided by:

ΔW=∈(νh^{T}−ν′h′^{T})

In addition, a step (vi) may include updating the biases a and b in an analogous manner, e.g., as provided by:

Δα=∈(ν−ν′) and Δb=∈(h−h′).

The hidden layer defines the latent space, and candidate amino acids sequences are generated by choosing values of the nodes of the hidden layer, and applying the RBM to decode the hidden layer values to an amino acid sequence, as the candidate sequence. Variations on this method can also be used, as discussed in Tubiana et al., “Learning protein constitutive motifs from sequence data,” eLife, vol. 8, p. e39397 (2019), which is incorporated herein by reference in its entirety.

General Description of Method

Returning to FIG. 3A, in step 120 of method 10, candidate points are selected within the latent space. For example, k-means clustering can be used to identify a region/neighborhood within the latent space that is likely to correspond to proteins having the desired functionality/attributes. Alternatively, a statistical analysis can be performed to determine a probability density function (pdf) for the desired functionality/attributes. A random number generator can then be used to select a statistically representative sample of points within the latent space. Other methods can also be used to determine samples of points within the latent space that are likely to correspond to proteins with the desired functionality.

In step 130 of method 10, the machine learning model 115 is used to select candidate amino acid sequences. These candidate sequences are selected based on their similarity to those sequences in the training dataset. For example, the training dataset can have a subset that in particular exhibit the desired functionality, and this subset can be clustered to identify a particular neighborhood of the latent space. Then, the candidate sequences can be selected based on the identified neighborhood. For example, because the machine-learning model 115 is a generative model, points within the identified neighborhood or in close proximity to the identified neighborhood can be mapped to amino acid sequences which are used as the candidate sequences.

The search for better performing amino acid sequences can have competing/complementary objectives of exploration and exploitation. In view of this, the choice of how much to deviate/differ from those amino acid sequences that are identified as high performers can depend on whether exploration or exploitation is more desired at a given stage of the search.

In step 140 of method 10, genetic sequences are synthesized, which are then used to generate proteins having the candidate amino acid sequences from step 130. The generated proteins are then assayed/evaluated to determine their functionality/attributes. Values representing the functionality/attributes of the generated proteins are then passed to step 150, in which a fitness function is generated to guide the selection of future candidate sequences.

The synthesis and assaying methods are described in more detail herein.

In step 150 of method 10, a fitness function is determined from the values measured in step 140. For example, the fitness function can be determined by performing, in the latent space, a regression analysis on the measured values. In certain implementations, Gaussian process regression is used to generate the fitness function from the measured values. Other non-limiting examples of methods for determining the fitness function are discussed herein.

In process 160 of method 10, the optimization search for candidate sequences continues using an iterative loop, as shown in FIGS. 2 and 3.

In step 162 of process 160, the machine-learning model is updated using the candidate sequences generated in step 130 or in a previous iteration of the loop in process 160. By expanding the number of amino acid sequences in the training dataset the machine-learning model 115 can be further trained to refine and improve the performance of the machine-learning model 115.

In step 164 of process 160, new candidate sequences are selected based on the machine-learning model 115 and the fitness function.

In step 166 of process 160, genetic sequences are synthesized and then used to generate proteins having the new candidate sequences from step 164. The generated proteins are then evaluated to measure new values representing their desired functionality. For example, the generated proteins can be assayed to measure desiderata corresponding to functionality.

In step 168 of process 160, various stopping criteria can be evaluated to determine if the stopping criteria are satisfied. For example, the stopping criteria can include whether the number of iterations exceeds or equals a predetermined maximum number of iterations. Additionally/alternatively, the stopping criteria can include whether the functionality of a predetermined number of candidate sequences exceeds a predefined functionality threshold. Additionally/alternatively, the stopping criteria can include whether, from iteration to iteration, the rate of improvement for the functionality of the candidate sequences has slowed down or converged, such that the rate of improvement has fallen below a predefined improvement threshold.

If the stopping criteria are satisfied, process 160 proceeds to step 172, in which the sequence(s) of the highest functioning protein candidate(s) are stored and/or presented to a user. Otherwise, process 160 proceeds to step 170.

In step 170 of process 160, the fitness function is updated using the new measured values from step 170. For example, the fitness function can be updated using a regression analysis on the new measured values. The regression analysis performed can use all of the measured values for all of the candidate sequences for the current iteration and all previous iterations.

The order of steps 162, 164, 166, 168, and 170 can be varied without deviating from the spirit of process 160. For example, the inquiry into whether the stopping criteria are satisfied can be performed between a different pair of steps other than steps 166 and 170. Further, step 162 can be omitted in certain iterations. For example, the fitness function can be updated for respective iterations of the loop in process 160 and the machine-learning method 115 can be held constant. By refining the fitness function from iteration to iteration the landscape of the desired functionality as a function of the latent space can be learned and improved to better select candidates with the desired functionality.

Further, the parameters for selecting the candidates can vary between iterations. For example, early in the search exploration can be favored relative to exploitation. Then, the candidates can be selected from a wider distribution. This strategy can be helpful for avoiding getting stuck in a local maximum of the functionality landscape. That is, the functionality landscape has peaks and values, and a global optimization method that encourages exploration can avoid iterating to amino acid sequences in a peak that is a local maximum, but is not a global maximum. One example, of a global optimization method is simulated annealing.

Other factors besides the functionality landscape can be important for selecting optimal candidates for the amino acid sequence. For example, these factors can include similarity and stability. Of the millions of possible sequences generated from the latent space sampling, an optimal selection method will focus on those that are most likely to have desirable properties. In effect, iterative process 160 performs in silico mutagenesis. That is, process 160 performs computational natural selection on those sequences generated by the unsupervised model to pick those predicted to be stable and highly functional. This is realized by each sequence generated by the latent space decoding being assigned to a score associated with its desirability.

In certain implementations, the selection of candidate sequences is performed using a scoring vector comprising three components: (i) similarity in terms of proximity to a known natural sequence—new sequences close to natural sequences are anticipated to stand a higher chance of preserving function, (ii) stability predicted by computational modeling using a software tool that predicts the protein structure—sequences predicted to preserve the native structure of the natural fold are likely to be more functional, and (iii) functionality as measured by an experimental assay (e.g., the measured values from step 166). The first two scores (i.e., similarity and stability) can be determined computationally for each predicted sequence in a high-throughput fashion. In certain implementations, the third score is determined approximately by fitting a supervised regression model over all proteins that have been previously synthesized and experimentally assayed (e.g., in step 140 or in step 166). The supervised regression model can be thought of as a landscape in the latent space. The supervised learning model can be fit using, but not limited to, multivariable linear regression, support vector regression (SVR), Gaussian process regression (GPR), random forests (RFs), and artificial neural networks (ANNs). By scoring each predicted sequence along these three measures, those sequences can be rank ordered, and those sequences anticipated to be most promising can be chosen for experimental synthesis.

For example, candidate sequence selection can be performed by identifying the Pareto frontier in this 3D multidimensional optimization space and selecting those sequences on the frontier as those proposed for synthesis. In certain implementations, the subset of sequences lying on the frontier may be further refined down by assigning adjustable weights to the three optimization criteria. For example, high weights associated with stability and with homology to a known natural sequence can present more conservative candidate sets, whereas low weights allow for more ambitious candidates further from natural sequences. That is, these low weights for similarity and stability can favor exploration. As the model becomes more accurate through multiple iterations, there tends to be greater confidence in the model, enabling the selection of more ambitious sequences further from nature.

For example, the goal of the proteins might be to exhibit the desired functionality within non-natural environments (e.g., high temperature, high pressure). In which case, it might be reasonable to assume that the protein structure would deviate from what nature has selected as optimal at room temperature and pressure. That is, more ambitious sequences might be advantageous in order to “computationally evolve” sequences towards functionality within non-natural environments (e.g., high temperature, high pressure) valuable in industrial applications but which would never be selected for in nature, which operates largely at room temperature and pressure.

Furthermore, the regression model can account for the uncertainty in the predictions of functionality. These uncertainties can be used within an exploit-explore modality in order to balance the competing interests in synthesizing the most promising sequences identified by the model with the interests in synthesizing sequences to explore the search space where the model has high uncertainty and new experimental data will contribute most improving the model.

Process 160 advantageously uses feedback to refine and improve the predictive capability of the fitness function together with the machine-learning model 115. For example, the sequences produced and assayed in steps 140 and 166 will be fed back into the supervised and unsupervised learning models (e.g., the fitness function and the machine-learning model 115). The machine-learning model 115 can learn a better latent space representation of a greater diversity of proteins. Further, the fitness function will become a better predictor of function. In this way the computational model becomes increasingly stronger in each iterative round of sequence manufacture and testing. Additionally, a data-driven representation can be used in an iterative process incorporating new sets of candidate proteins to automatically build a larger implicit library, and then use the smaller set of labeled examples to build a regression model from the continuous representation to the desired properties.

Gene Synthesis

Regarding the gene synthesis in steps 140 and 166, a process can be used to synthesize genetic sequences for the candidate amino acid sequences using tube-synthesized (high-purity) oligonucleotides and automated bacterial cloning techniques. However, this process is generally expensive. Accordingly, improved, cheaper gene synthesis processes are desired.

One less expensive approach (e.g., producing ˜500 bp long genes at large scale, i.e., thousands of sequences, with an amortized cost of $˜2/gene) uses beads functionalized with oligonucleotide barcodes to segregate copies of all oligonucleotides required for a given gene into a single droplet of a water-in-oil emulsion, followed by polymerase cycling assembly (PCA) of the overlapping oligonucleotides into the full length gene, as described in W. Stemmer, et al., “Single-Step Assembly of a Gene and Entire Plasmid From Large Numbers of Oligodeoxyribonucleotides,” Gene. 164 (1995) 49-53 and in C. Plesa, et al., “Multiplexed gene synthesis in emulsions for exploring protein functional landscapes,” Science. 359 (2018) 343-347, both of which are incorporated herein by reference in their entirety. This approach can produce, e.g., —500 bp long genes at large scale (i.e., thousands of sequences) with an amortized cost of $˜2/gene, albeit with large error rates (e.g., —5% correct amino acid sequence). This method is referred to herein as the bead-and-barcode method.

Another even less expensive approach starts with longer oligos and uses separate minipools, thereby omitting the need for bead hybridization of the bead-and-barcode method. This method is referred to herein as the minipool method. In the minipool method, pre-commercial oligonucleotides are synthesized in array format with two significant improvements over previously available products. First, the pre-commercial oligonucleotides are synthesized with length of up to 300 nt. Second, the pre-commercial oligonucleotides are synthesized with an error rate of ˜1:1300. This increase in length (as compared with a length of 200 nt available previously) decreases the complexity of the assembly reaction, as 60-80 nt of each oligonucleotide are used for ‘overhead’ sequences that do not contribute to the final gene product. The effective sequence available per oligonucleotide thus increases by 75% (from ˜130 nt to ˜230 nt) for 300-nt oligonucleotides, allowing assembly of 1 kb genes with a similar number of oligonucleotides as was used with the previously available pre-commercial oligonucleotides to make 500 bp genes.

The lower error rate results in fewer sequence errors in the assembled genes, as single-base insertions and deletions in the oligonucleotides are the dominant source of sequence errors. Additionally, by providing the oligonucleotides as separate ‘minipools’ containing only the oligonucleotides needed for each gene, the bead hybridization step of the bead-and-barcode method can be omitted, with a concomitant reduction in cost and complexity.

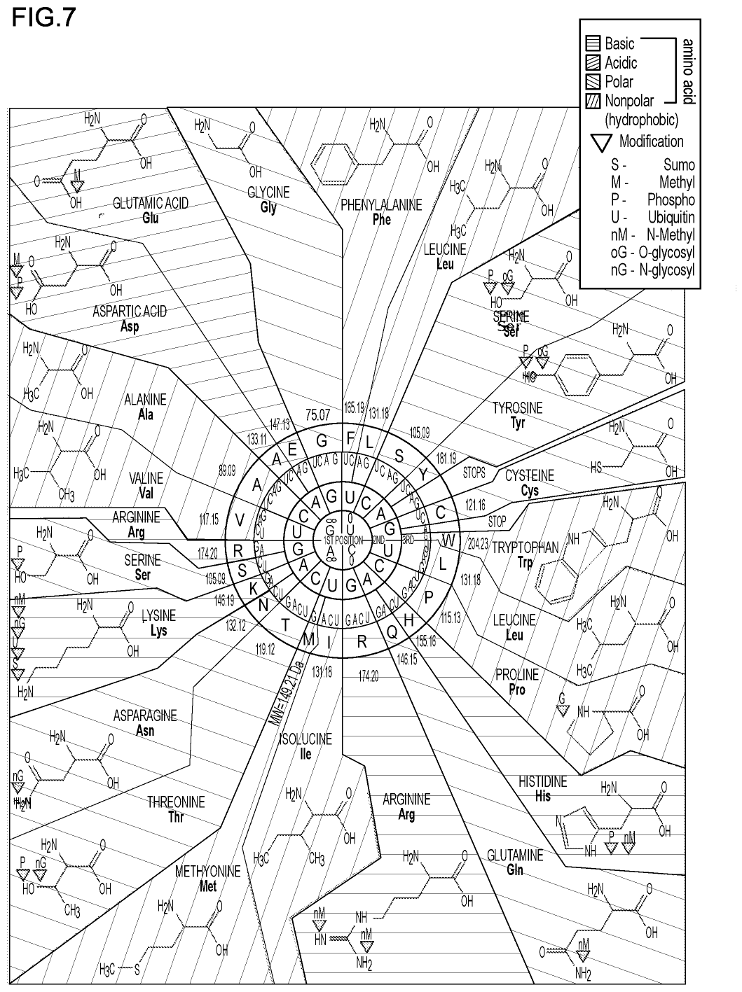

Errors in oligonucleotide synthesis are not randomly distributed but are linked to their sequence. For example, purine bases are more susceptible to degradation during synthesis than pyrimidines, and formation of compact folded structures by the growing oligonucleotide chain hinders addition of subsequent nucleotides. The flexibility of the genetic code (multiple codons encode the same amino acid, as shown in FIG. 7) should allow the design of oligonucleotides with improved synthesis accuracy. The choice of which codon to use in order to encode a particular amino acid can be guided by measuring the accuracy of the delivered oligonucleotides using high-throughput (HT) sequencing, identifying sequence patterns linked to poor performance, and updating the oligonucleotide design algorithm to avoid them.

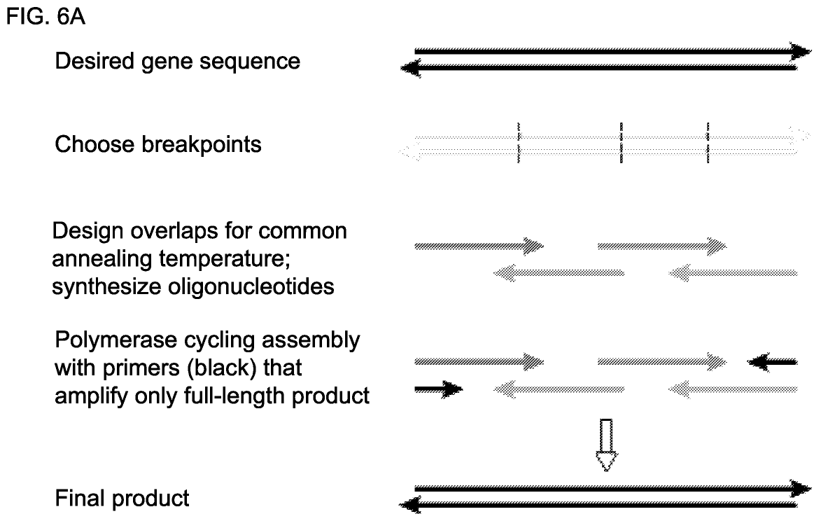

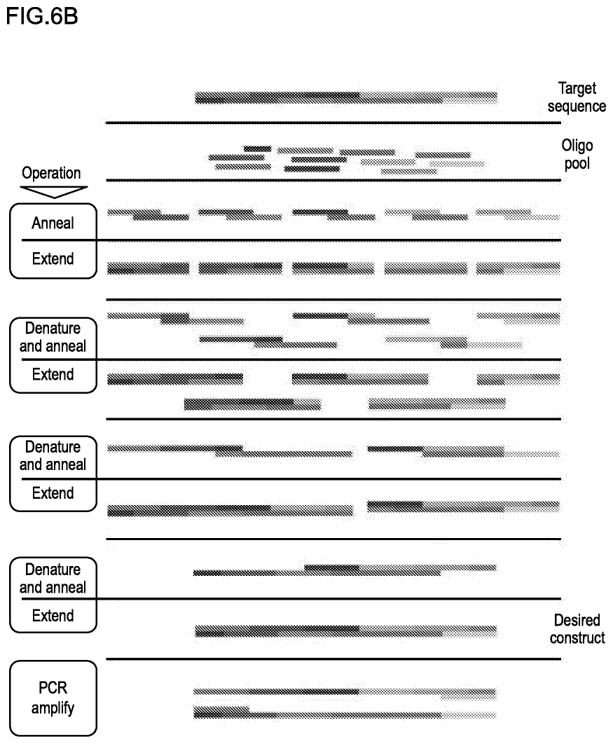

Regarding the optimization of polymerase cycling assembly (PCA), PCA generates/synthesizes genetic sequences that express the candidate amino acid sequences by assembling oligonucleotides into a larger gene by annealing overlapping sequences followed by polymerase extension, analogous to polymerase chain reaction (PCR) technique but with the overlap sequences in the role of primers, as shown in the schematic diagrams shown in FIGS. 6A and 6B. As a result, design of the overlap sequences is significant for successful assembly, in the same way that good primers are required for successful PCR amplification. Overlap sequences are orthogonal to the other overlaps in the gene, but also anneal at a similar temperature to one another. The amino acid sequence of the gene limits the freedom to choose the overlaps but does provide limited freedom as described in the previous section. Additionally, the breakpoints can be chosen between oligonucleotides in order to optimize both of these parameters (i.e., annealing temperature and orthogonality between overlaps) to achieve high efficiency assembly.

Variations for the method of genetic sequence synthesis are within the spirit of the methods disclosed herein. For example, gene synthesis can be performed using ligase chain reaction methods, thermodynamically balanced inside out synthesis, and gene synthesis by ligation, as well as various error correction methods (e.g., chew back, anneal, and repair).

Direct Coupling Analysis (DCA) Model

As discussed above, the method 10 can be performed using different types of machine-learning models 115 (also referred to in certain embodiments as an unsupervised-learning model 115). Now, a non-limiting embodiment is provided using a DCA for the unsupervised-learning model 115.

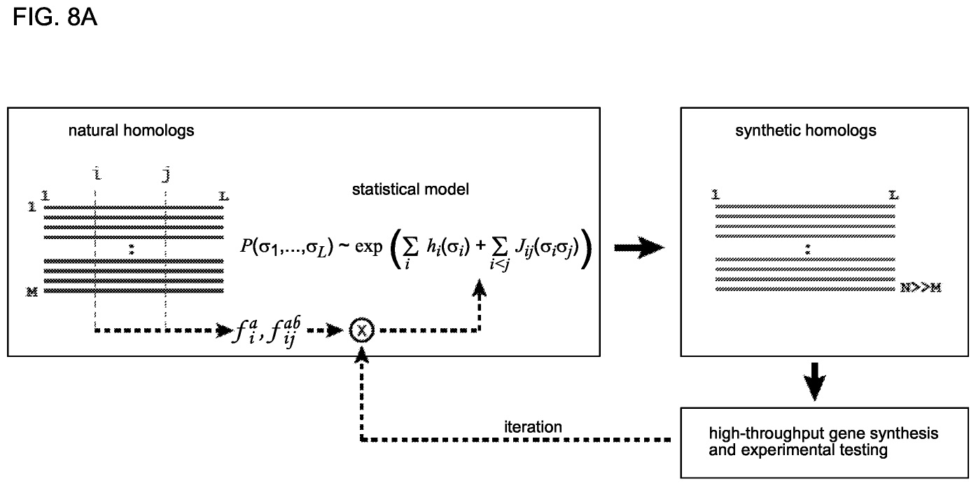

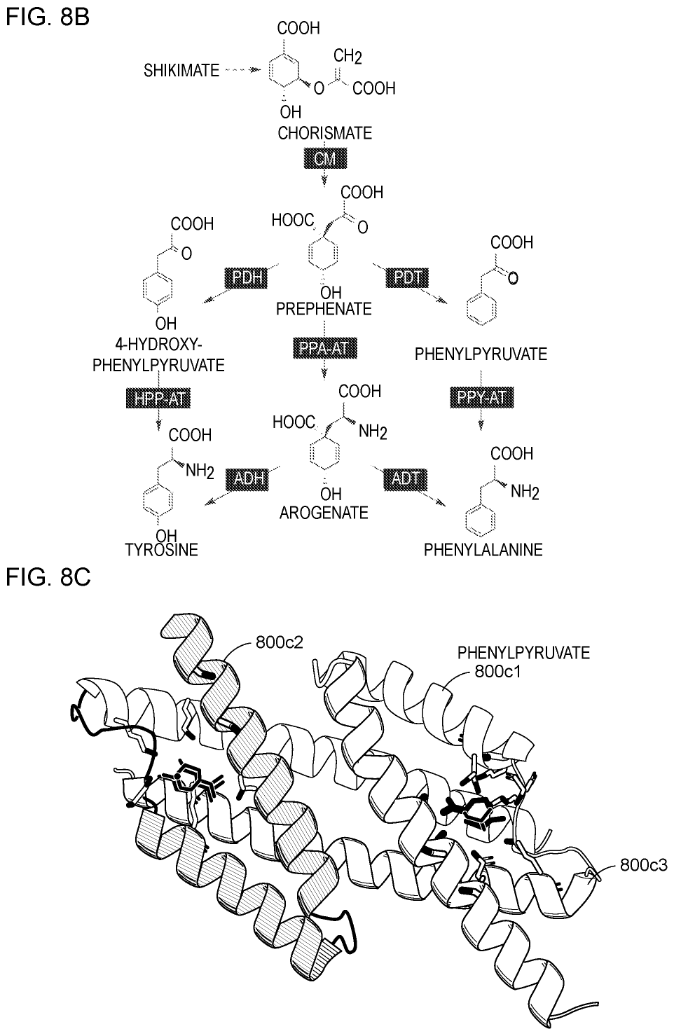

An approach for evolution-inspired protein design is shown in FIG. 8A, based on direct coupling analysis (DCA), a method originally conceived to predict contacts between amino acids in protein three-dimensional structures. In general, the algorithm begins with a multiple sequence alignment of natural homologs, from which the empirical first and second-order statistics are computed. These quantities are used to learn a minimal statistical model comprised of intrinsic constraints on amino acids (hᵢ) and pairwise interactions (Jᵢⱼ). Next, the statistical model can be used to generate many more artificial sequences that recapitulate the natural statistics and can be screened for desired activities.

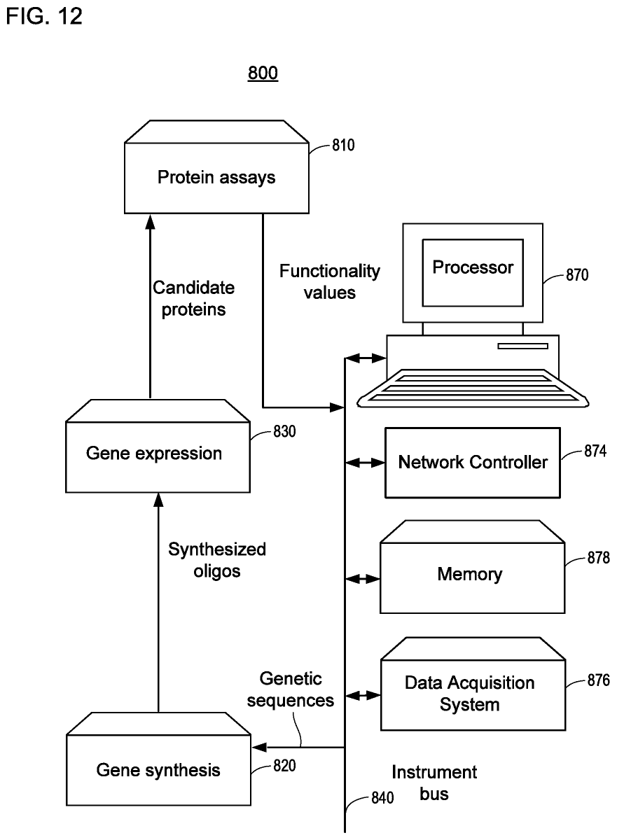

FIG. 12 shows a portion of the shikimate pathway in bacteria and fungi, leading to the biosynthesis of the aromatic amino acids tyrosine and phenylalanine; the AroQ family of chorismate mutases (CMs) operates at a branch point.

FIG. 8C shows an atomic structure of E. coli CM, a dimer (e.g., items 800c1, 800c2, and 800c3) with two functional active sites. Each active site is comprised of amino acids contributed by both protomers. A bound substrate analog is shown in magenta stick bonds.

The starting point is a large and diverse multiple sequence alignment (MSA) of a protein family, from which the observed frequencies (ƒᵢ^{α}) and pairwise correlations (ƒᵢⱼ^{ab})ƒᵢⱼ^{ab}) of all amino acids are estimated—the first- and second-order statistics. From these quantities, a model is inferred comprising a set of intrinsic amino acid propensities (fields hᵢ) and a minimal set of pairwise interactions (couplings Jᵢⱼ) that optimally account for the observed statistics. This model is defined as:

P(σ₁, . . . ,σ_{L})˜exp[−H(σ₁, . . . ,σ_{L})],

In the above model P is the probability an amino acid sequence (σ₁, . . . , σ_{L}) to occur, L is the length of the protein and H (σ₁, . . . ,σ_{L})=Σᵢ hᵢσᵢ+Σ_{i<j}Jᵢⱼσᵢσⱼ is a statistical energy (or Hamiltonian) that provides a quantitative score for the likelihood of each sequence. Lower energies are associated with higher probability, allowing us by Monte Carlo sampling to generate non-natural sequence repertoires, which can then be screened for desired functional activities. If pairwise correlations suffice in general to capture the information content of protein sequences and if the model inference is sufficiently accurate, the synthetic sequences should recapitulate the functional diversity and properties of natural proteins.

This DCA implementation of method 10 is now illustrated using a non-limiting example of training the DCA unsupervised-learning model 115 using a multiple-sequence alignment (MSA) of chorismate mutases (CM) homologs.

To illustrate method 10 using a DCA as the sequence model, the method was performed using the AroQ family of chorismate mutases (CMs), a classic model for understanding principles of catalysis and enzyme design. These enzymes can be found in bacteria, plants, and fungi and operate at a branch-point of the shikimate pathway leading to the biosynthesis of tyrosine and phenylalanine (as illustrated in FIG. 12). CMs catalyze the conversion of the intermediary metabolite chorismate to prephenate through a Claisen rearrangement, displaying more than a million-fold rate acceleration of this reaction, and are necessary for growth in bacterial cells. For example, E. coli strains lacking CM are auxotrophic for tyrosine and phenylalanine, with both the degree of supplementation of these amino acids and the expression level of CM quantitatively determining the growth rate. Structurally, AroQ CMs form a domain-swapped dimer of relatively small protomers (˜100 amino acids, FIG. 1e) which, together with requirement for bacterial growth and the existence of good biochemical assays, makes them an excellent design target for testing the power of statistical models inferred from MSAs.

First, an MSA including a large number of sequences is created. In one implementation, sequences are acquired using residues 1-95 of the E. coli P-protein as the starting query for 3 rounds of PSI-Blast (ref) and an e-score cutoff of 1e-4. An initial alignment is generated, starting with a structural alignment of PDB entries 1ECM, 2D8E, 3NVT and 1YBZ and iteratively generating an alignment profile and aligning nearest neighbor sequences from the PSI-Blast results to this profile using muscle (ref). The resulting alignment is adjusted and trimmed to the regions seen in 1ECM, to remove short sequences (less than 82 residues), to remove sequences that added poorly represented gaps (<30% occupied) and to reduce redundancy (>90% top-hit identity). An MSA of 1,259 sequences is created in this manner, which serves as input for sequence design and for testing the experimental procedure on functional sequences. Other MSAs of other homologs can be generated in a similar manner with variations to the procedure, as would be understood by a person of ordinary skill in the art.

Next, the DCA analysis is performed using the MSA. For example, the MSA can be used to infer a Potts model, assigning a probability, e.g.:

$$P ( {a}_{1} , … , {a}_{L} ) = \frac{1}{Z} exp \{ - β H ( {a}_{1} , … , {a}_{L} ) \}$$

Such probability may be assigned to each aligned sequence |(α₁, . . . ,α_{L}) of L=95 amino acids or alignment gaps. The statistical energy (or Hamiltonian) may be provided by:

$$H ( {a}_{1} , … , {a}_{L} ) = - {\sum }_{1 \leq i < j \leq L} {J}_{ij} ( {a}_{i} , {a}_{i} ) - {\sum }_{1 \leq i \leq L} {h}_{i} ( {a}_{i} )$$

The statistical energy (or Hamiltonian) of the Potts model is given in terms of the direct coevolutionary couplings Jᵢⱼ(a, b) between amino acids a and b in positions i and j, and biases (or fields) hᵢ (α) for the usage of amino acid a in position i. These parameters are inferred using bmDCA (reweighting threshold 0.8, regularization strengths 10⁻² and 10⁻³). The formal inverse temperature, e.g., as provided by |β=1/T, is set to one during inference.

The aim of the resulting model is to accurately reproduce the empirical fraction ƒᵢ (α) of natural sequences having amino acid a in position i, and the fraction ƒᵢⱼ (a, b) of sequences having simultaneously amino acids a and b in positions i and j, thus bringing residue conservation and covariation together, e.g.:

$${f}_{i} ( {a}_{i} ) = {\sum }_{\{ {a}_{k} \vert k \neq i \}} P ( {a}_{1} , … , {a}_{L} ) , \\ {f}_{ij} ( {a}_{i} , {a}_{j} ) = {\sum }_{\{ {a}_{k} \vert k \neq i , j \}} P ( {a}_{1} , … , {a}_{L} ) .$$

To check the accuracy of the inferred model, the sequence statistics of natural sequences is compared with MCMC (Markov chain Monte Carlo) samples drawn from the formula: |P(α₁, . . . , α_{L}). This may be performed using connected two-residue and three-residue correlations, e.g., as provided by the following formulas [1] and [2], respectively:

Cᵢⱼ(a,b)=ƒᵢⱼ(a,b)−ƒᵢ(α)ƒⱼ(b) [1]

Cᵢⱼₖ(a,b,c)=ƒᵢⱼₖ(a,b,c)−ƒᵢⱼ(a,b)ƒₖ(c)−ƒᵢₖ(a,c)ƒⱼ(b)−ƒⱼₖ(b,c)ƒᵢ(α)+2ƒᵢ(α)ƒᵢ(b)ƒₖ(c) [2]

Formulas [1] and [2], as referenced above, describe the part of the empirical two—and three-residue fractions, respectively, which are not explainable via lower-order statistics. They are thus intrinsically harder to reproduce than ƒᵢⱼ (a, b) and ƒᵢⱼₖ(α, b, c) and constitute a more stringent test of the accuracy of the model.

Regarding the use of regularization, the statistical model |P(αᵢ, . . . , α_{L}) depends on a multitude of parameters {j, h}, which are inferred from finite data. To avoid strong overfitting effects, DCA can use L2-regularization, i.e. a penalty, which may be represented by, e.g.:

$$λ ( {\sum }_{1 \leq i < j \leq L} {\sum }_{ab} {{J}_{ij} ( a , b )}^{2} + {\sum }_{1 \leq i \leq L} {\sum }_{a} {{h}_{i} ( a )}^{2} )$$

In this way, a penalty value, e.g., as output by the above formula, may be added to the likelihood of the data. This penalty systematically decreases the parameter values in bmDCA inference, and thereby avoids extremely large parameter values due to under-sampled rare events. This modifies the consistency equations between model |P(αᵢ, . . . , α_{L}) and the empirical frequency counts into the following formulas:

$${f}_{i} ( {a}_{i} ) = {\sum }_{\{ {a}_{k} \vert k \neq i \}} P ( {a}_{1} , … , {a}_{L} ) + λ {h}_{i} ( {a}_{i} )$$

$${f}_{ij} ( {a}_{i} , {a}_{j} ) = {\sum }_{\{ {a}_{k} \vert k \neq i , j \}} P ( {a}_{1} , … , {a}_{L} ) + λ {J}_{ij} ( {a}_{i} , {a}_{j} )$$

If the statistical energies (H(α₁, α_{L})) of natural sequences (NAT— frequency counts ƒ) are compared with the ones sampled from the inferred model at |β=1(MCMC—frequency counts given by P), it is observed that mean energies [symbol] H [symbol] are systematically shifted, e.g., as provided by the following formula:

$${〈 H 〉}_{NAT} = {〈 H 〉}_{MCMC} - λ ( {\sum }_{1 \leq i < j \leq L} {\sum }_{a b} {{J}_{ij} ( a , b )}^{2} + {\sum }_{1 \leq i \leq L} {\sum }_{a} {{h}_{i} ( a )}^{2} ) ,$$

That is, in the model, natural sequences have systematically lower energies, and thus higher probabilities, than sampled sequences, e.g., as represented by:

[symbol]

H [symbol] _{NAT}< [symbol] H [symbol] _{MCMC}

To overcome this gap, lower temperatures T<1 are introduced, forcing MCMC to sample at lower statistical energies, which are compatible to the natural sequences.

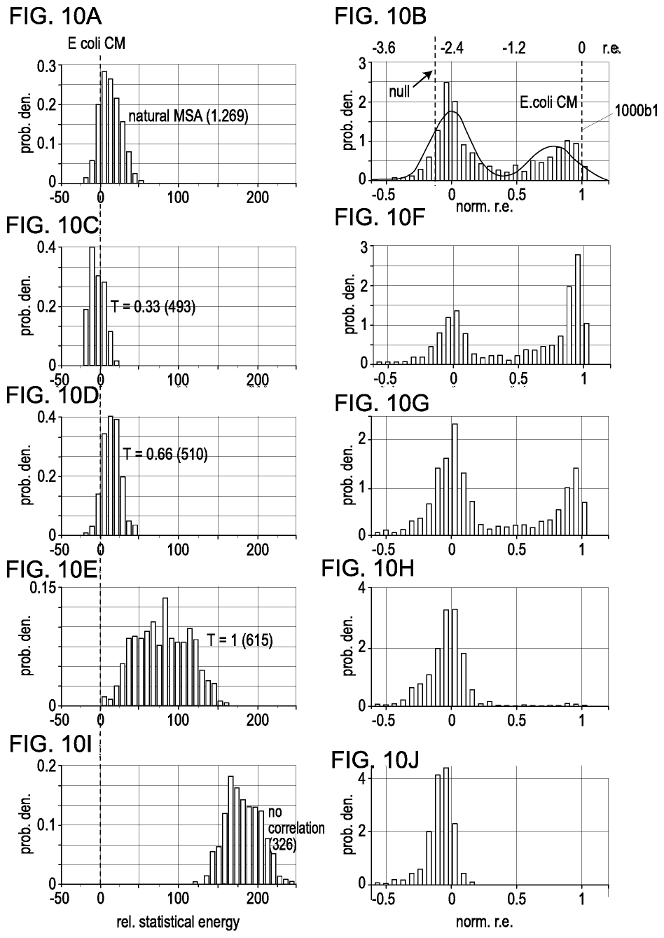

Once the DCA model is trained, the DCA model can be used to generate new candidate sequences. MCMC sampling is used to generate sequences from |P(αᵢ, α_{L}), MCMC. The temperature T in the Pott's model (i.e., β=1/T) controls the breadth of the sampling. For example, the temperature can be increased to enlarge the range of sampled energies in particular to lower energies H(αᵢ, . . . , α_{L}), and thus higher probabilities P(αᵢ, . . . , α_{L}). As an illustrative example, FIGS. 10C-E show experimental results for samples taken respectively at temperatures T∈{0.33,0.66,1.0}.

DCA was used to make a statistical model for an alignment of 1259 natural AroQ CM enzymes that broadly encompasses the diversity of bacterial and fungal lineages. Technically, deducing the parameters (hᵢjᵢⱼ) from the observed statistics in the MSA ƒᵢ, ƒᵢⱼ) for any protein is computationally intractable by direct means, but a number of approximation algorithms are possible. Here, the bmDCA approximation algorithm is used, a computationally expensive but highly accurate method based on Boltzmann machine learning. In other implementations, the parameters (hᵢ, Jᵢⱼ) for the DCA model can be obtained using a mean-field solution method, a Monte Carlo gradient descent method, or a pseudo-likelihood maximization method.

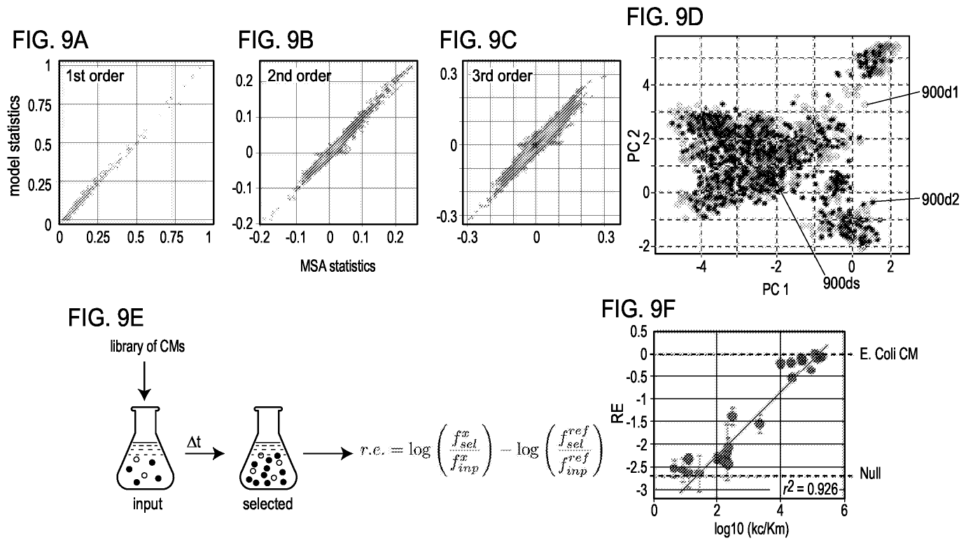

FIGS. 9A and 9B show sequences sampled from the bmDCA model recapitulate the empirical first- and second-order MSA statistics, respectively. It can be observed from these results that the model was well-fit. FIG. 9C shows that the bmDCA model also recapitulates third-order correlations in the MSA that were not used in training the model, suggesting that the model is statistically complete.

FIG. 9D shows the top two principal components of the distance matrix between all natural CM sequences in the MSA (circular items or points shaded as, e.g., item 900d1). Here, the structure of the sequences space spanned by the CM family are visualized. The sequences derived from the bmDCA model (circular items or points shaded as, e.g., item 900d2) also fill the sequence space in a manner consistent with natural CM sequences. The position of the E. coli CM sequence is indicated by point 900ds.

Accordingly, it is apparent that sequences generated by Monte Carlo sampling from the model reproduce the empirical first- and second-order statistics of natural sequences used for fitting. The statistics are shown in FIGS. 9A, 9B, and 9C, showing the first-, second—and third-order statistic respectively. Additionally, it can be observed that the model recapitulates higher order statistical features in the MSA that were never used in inferring the model. This includes three-way residue correlations (see FIG. 9C) and the inhomogeneously clustered phylogenetic organization of the protein family in sequence space (see FIG. 9D), suggesting that the statistical model captures the essential rules governing the divergence of natural CM sequences through evolution. In contrast, a simpler model that retains only the intrinsic propensities of amino acids at sites (hᵢ) and leave out pairwise couplings fails to reproduce even the second-order statistics of the MSA, and does not account for the pattern of sequence divergence in the natural CM proteins.

This example illustrates that bmDCA provides a statistical model for generating new candidate sequences, meaning that natural sequences and sequences sampled from the probability distribution P(σ) are, despite considerable sequence divergence, functionally equivalent. To illustrate this, a high-throughput, quantitative in vivo complementation assay for CM in E. coli is used to evaluate candidate proteins generated using the bmDCA model for a desired functionality. Here, catalytic power is used as the desired functionality to illustrate the method. The high-throughput, quantitative in vivo complementation assay is suitable for studying large numbers of natural and designed CMs in a single internally-controlled experiment.

FIG. 9E shows a quantitative high-throughput functional assay for CM in which libraries of CM variants are expressed in an E. coli strain deficient for chorismate mutase, grown as a mixed population under selective conditions, and then subject to next generation sequencing to count the frequency of each CM allele in the input and selection populations.

FIG. 9F shows that a relative enrichment (r.e.) can be computed from the measurements of the quantitative high-throughput functional assay. The r.e. provides indicia of the catalytic power (e.g., the desired functionality) because the r.e.is close to linear with catalytic power (ln(kc/Km)) over a roughly five log-order range. This “standard curve” was made using a panel of point mutants of E. coli CM that span a broad range of specific activities.

Now a brief description of the high-throughput assay is provided, and a more detailed description is provided below. Libraries of CM variants (natural and/or synthetic, e.g., in a cold start) are made using a custom de novo gene synthesis protocol that is capable of fast and relatively inexpensive assembly of novel DNA sequences at large scale. For example, libraries comprising every natural CM homolog in the MSA (1,259 total) were made, and more than 1,900 synthetic variants were made to explore various design parameters of the bmDCA model. These libraries were expressed in a CM-deficient bacterial strain (KAl2) and grown together as a single population in selective media lacking phenylalanine and tyrosine to select for chorismate mutase activity (as shown in FIG. 9E). Deep sequencing of the population before and after selection allows us to count the log frequency of each allele relative to wild-type, a quantity called the “relative enrichment” (r.e.) which under particular conditions of induction, growth time, and temperature, quantitatively and reproducibly reports the catalytic activity of chorismate mutase (as shown in FIG. 9F). This “select-seq” assay is near-linear over a broad range of catalytic power and serves as an effective tool to rigorously compare large numbers of natural and synthetic variants for functional activity in vivo, in a single internally controlled experiment.

The first study is to examine the performance of the natural CM homologs in the select-seq assay, a positive control for bmDCA designed sequences. Natural sequences show a unimodal distribution of bmDCA statistical energies centered close to the value of the E. coli CM (defined as zero, see FIG. 10A), but it is not obvious how they will function in the particular E. coli strain and experimental conditions used in the assay. For example, members of the CM family may vary in unknown ways with regard to activity in any particular environment, and the MSA includes some fraction of paralogous enzymes that carry out a related but distinct chemical reaction. The select-seq assay shows that the 1,259 natural CM homologs in the MSA exhibit a bimodal distribution of complementation in the assay, with one mode comprising ˜ 31% of sequences centered at the level of wild-type E. coli CM, and the remainder comprising a mode centered at the level of the null allele (see FIG. 10B). A green fluorescent protein (GFP) tagged version of the library suggests that the bimodality of complementation is not obviously related to differences in expression levels compared to the E. coli variant; instead, the bimodality presumably originates from the cooperativity of amino acids in specifying catalytic power for chorismic acid, and from the possibility of paralogous sequences. For the purpose of this study, the bimodality allows the reduction of the full distribution to simply the probability that sequences complement function in the assay. Importantly, the standard curve shows that this quantity is a stringent test of high chorismate mutase activity (see FIG. 9F).

To evaluate the generative potential of the bmDCA model, Monte Carlo sampling is used to randomly draw sequences from the model that span a range of statistical energies relative to the natural MSA. For example, FIGS. 10C-E illustrate that sequences with low energy will be functional chorismate mutases.

Regarding the sampling process, it should be noted that sequence data are inherently limited (for instance, not all amino acids occur at every position) and even large sequence families are under-sampled relative to the number of combinations of all amino acids at all pairs of sites in the MSA. In view of these limitations, to avoid overfitting, regularized inference is used in making the bmDCA model. The use of regularization causes sequences sampled from the model to have, on average, smaller probabilities (i.e. higher statistical energies) than the natural sequences. To sample sequences with low energy, a formal computational “temperature” T<1 is introduced in a model, e.g., as provided by:

$${P}_{T} ( {σ}_{1} , … , {σ}_{L} ) ∼ exp ( \frac{- H ( {σ}_{1} , … , {σ}_{L} )}{T} )$$

Such model compensates for the effect of regularization. For example, sampling at T□{0.33,0.66} produces sequences with statistical energies that more closely reflect the natural distribution, and that show little dependence on regularization parameters, for example as shown for FIGS. 10C and 10D. In contrast, sequences sampled at T=1 show a broad distribution of statistical energies that deviate significantly from the natural distribution and that depend more strongly on the strength of regularization, for example, as shown for FIG. 10E.

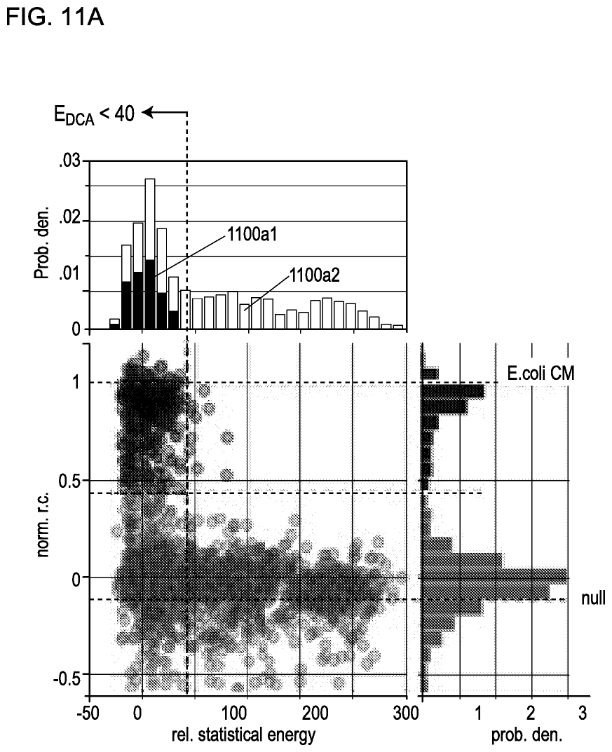

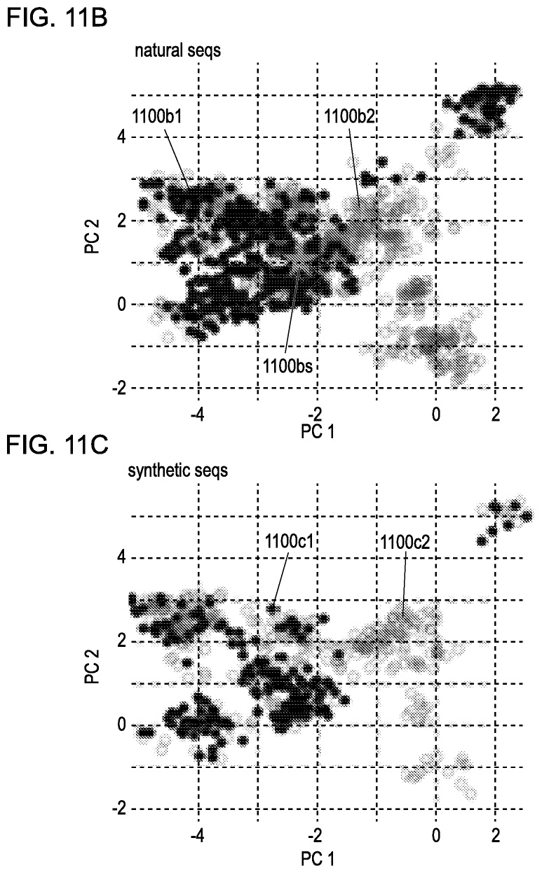

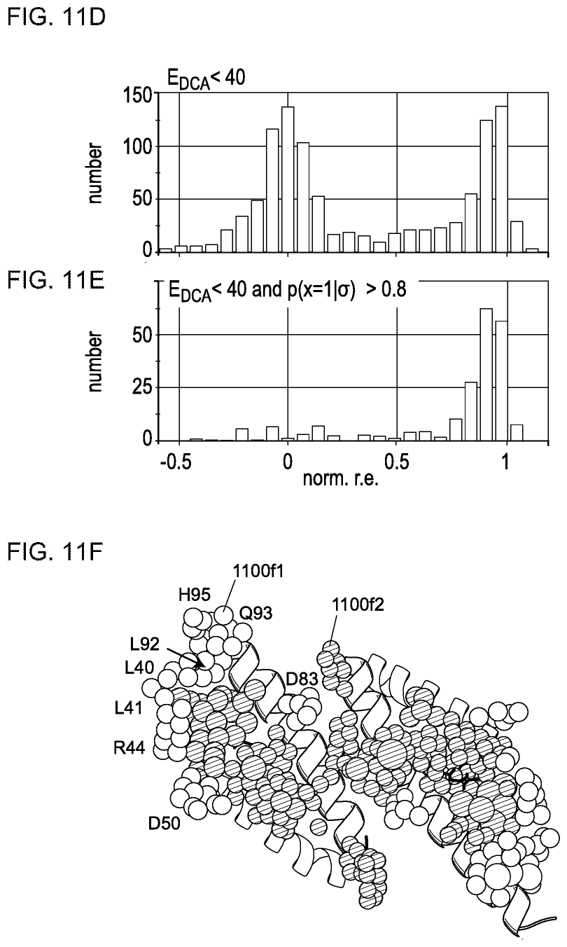

Libraries of 350 synthetic sequences were made and tested. These libraries sampled at T □{0.33,0.66,1.0} each from the bmDCA model. FIGS. 10F-H show that overall, these sequences also display a bimodal distribution of complementation, with many complementing function near to the level of the wild-type E. coli sequence. Consistent with the hypothesis, the probability of complementation is well-predicted by the bmDCA statistical energy, with low-energy sequences drawn from T □{0.33,0.66} essentially recapitulating or even somewhat exceeding the performance of natural sequences (FIG. 10f-g). In contrast, sequences drawn from T=1 show poor performance, consistent with deviation from the bmDCA model (see FIG. 10H). In total, 521 synthetic sequences out of 1,050 total (˜50%) rescue growth in the assay, comprising a range of top-hit identities to any natural chorismate mutase of 44-92%. These include 48 sequences with less than 65% identity to proteins in the MSA, corresponding to at least 33 mutations away from the closest natural counterpart. Sequence divergence from the E. coli CM ranges from 19 to 42%. A representation of the positions in the protein that contribute most to the bmDCA statistical energy highlights residues distributed both within the active site and extending through the CM tertiary structure to include the dimer interface (see FIG. 11F).

FIGS. 10A-10I show a functional analysis of natural and synthetic CM sequences. FIG. 10A shows the ensemble of natural CM sequences in the MSA comprise a unimodal distribution of bmDCA sequences centered close to the value for E. coli CM (defined as zero). FIG. 10B shows the relative energy (r.e.) scores for natural CMs shows a bimodal distribution, with one model comprising ˜ 31% of sequences near to the level of the E. coli CM (defined as zero r.e. or one in terms of normalized r.e. dashed line 1000b1), and the remainder of sequences near to the level of the CM null allele (red dashed line).

FIGS. 10C-10D show bmDCA statistical energies for sequences sampled at three different computational temperatures (0.33, 0.66, 1), respectively. Sequences sampled at temperatures 0.33 and 0.66 closely reproduce the energies of natural sequences, but sequences drawn at T=1 do not reproduce the energies of natural sequences.

In FIGS. 10F-10H, the functional analysis shows that sequences sampled at temperature T of 0.33 and 0.66 recapitulate or even exceed the performance of natural sequences, but that sequences sampled at T=1 are mostly non-functional.

FIGS. 10I and 10J show sequences made by preserving the first-order statistics but leaving out correlations show large statistical energies and show no function at all. Thus, the bmDCA model encodes natural-like function in synthetic CM sequences.

FIG. 11A shows a scatterplot of all synthetic CM sequences, showing the relationship between bmDCA statistical energy and catalytic function. Functional sequences (bars shaded as, e.g., item 1100a1) and non-functional sequences (bars shaded as, e.g., item 1100a2) are shown. The data show that function is predicted by low bmDCA energies, with essentially no sequences with E DCA <40 complementing the CM null phenotype.

FIG. 11B shows the top two principal components of sequence variation defined by the natural CM sequences as in FIG. 9D, indicated or otherwise shaded as in FIG. 11A. The E. coli sequence is marked by point 1100bs. These data show that natural CM sequences classified as functional in E. coli are localized to specific regions within the overall pattern of sequence variation.

FIG. 11C shows a projection of synthetic CMs onto the same space, showing that functional sequences are localized to the same clusters. The information regarding which regions of the PCA-defined domain the functional sequences are localized to/clustered in can be used to define a score (e.g., a functionality landscape) that is then used to bias the selection of the candidate sequences from regions corresponding to the clusters of functional sequences. For example, the Boltzmann distribution can define a probability density function according to which the candidate sequences are randomly drawn. This probability density function can be biased to increase the density of sequences drawn that correspond to the clusters of functional sequences. In FIGS. 11A-C the determination of functional versus non-functional is binary, but in other implementations a continuous scale of functionality can be used and GPR can be used, e.g., to generate a functionality landscape.

FIGS. 11D and 11E show the pattern of complementation for synthetic sequences with EDCA <40, either without (FIG. 11D) or with (FIG. 11E) an extra statistical condition derived from the pattern of functional complementation of natural CM sequences (P(x=1|σ)). The data show that prediction of synthetic sequences complementing function in a specific context can be dramatically enhanced using the prior knowledge of function in natural CMs.

FIG. 11F shows the structure of E. coli CM, with positions contributing most to low statistical energy shown in spheres (spheres or circles shaded as, e.g., item 1100f2), and positions contributing to E. coli specific function (spheres or circles shaded as, e.g., item 1100f1). The data show that the general design constraints focus on a physically contiguous pattern of residues both at the active sites and extending to the dimer interface, and that the extra constraints on E. coli-specific function are located more peripherally.

As another control, 326 sequences were produced with the same distribution of sequence identities as bmDCA designed sequences at T=0.66 but preserving only the first order statistics and leaving out the correlations. These sequences expectedly show high bmDCA energies and display no complementation at all (See FIGS. 101 and 10J), demonstrating that enzyme function fundamentally depends on the pattern of correlations imposed by the couplings in Jᵢⱼ and not just the magnitude of sequence variation.

Putting all the data together, a strikingly steep relationship can be observed between bmDCA statistical energy and CM activity—nearly 50% of designed sequences rescue the CM null phenotype when the statistical energy is below a threshold value set by the width of the energy distribution of natural sequences (EDCA <50) and essentially no sequences are functional above this value (See FIG. 11A). Thus, bmDCA is an effective generative model, capable of designing natural-like enzymatic activity with considerable sequence diversity if statistical energies are within the range of natural homologs.

The bmDCA model captures the overall statistics of a protein family and does not focus on specific functional activities of individual members of the family. Thus, just like natural CM homologs, most bmDCA designed sequences do not complement function in the specific conditions of the assay (See FIG. 11A). The generative model may be improved to deduce the extra information that makes a protein sequence optimal for a specific phenotype For example, sequences that rescue function the assay occupy the sequence space spanned by natural CM sequences. Natural CMs that complement function in E. coli are distributed in several diverse clusters (See FIG. 11B), but interestingly, functional synthetic sequences also follow the same pattern (See FIG. 11C). This shows that information about CM function in the specific context of E. coli and assay conditions exist in the statistics of natural sequences and might be learned. In such embodiments, knowledge gained in one experimental trial can be used to formally train computational models to predict synthetic sequences that encode particular protein phenotypes and organismal environments.

As a test of the above referenced embodiment, a DCA model was trained/generated from sequences in the natural MSA, but now annotated with a binary value x indicating their ability to function in the assay (x=1 of functional and is zero if not). From this model, a probability can be computed that any synthetic sequence a will complement function in the E. coli select-seq assay; that is, P(x=1|σ). FIGS. 11D and 11E show that for low-energy CM-like synthetic sequences from the naïve bmDCA model (See FIG. 11D), the extra condition that P(x=1|σ) now efficiently predicts the subset that complements in the context of the assay (83%, See FIG. 11e). Mapping the top positions that contribute significantly to E. coli-specific CM sequences shows a clustered arrangement of amino acids peripheral to the active site (See FIG. 11F). Thus, these positions work allosterically to control catalytic activity, a mechanism to provide context-dependent tuning of reaction parameters. These results support an iterative design strategy for specific protein phenotypes in which the bmDCA model is updated with each round of selection to optimally target desired phenotypes.

The results described here validate and extend the concept that pairwise amino acid correlations in practically-available sequence alignments of protein families suffice to specify protein folding and function. The bmDCA model is one approach to capture these correlations.

Now a more detailed description is provided of the non-limiting high-throughput gene construction and assays used for FIGS. 10A-J and FIGS. 11A-E discussed above. CM genes were built using PCR overlap extension of oligos synthesized on microarray chips. Two oligos (230-mer) were designed for each gene with a unique pair of flanking orthogonal primer annealing sites for “gene-specific primers” (GSPs) and a Btsal restriction site to remove the flanking region after amplification. Overlaps were designed to be at least 16 bases long, to have 3′ G or C bases, and to have a melting temperature of at least 59° C., calculated using a nearest-neighbor method, as discussed in Breslauer et al. “Predicting DNA duplex stability from the base sequence,” Proc Natl Acad Sci. vol. 83, pp. 3746-3750. (1986), which is incorporated herein by reference in its entirety. PCR was performed in 384-well plates using Q5 polymerase with 1× Q5 buffer, 0.2 μM dNTPs and a pair of 0.5 μM GSPs in 10 ul total volume. The oligos corresponding to an individual gene were amplified with 35 cycles of 10 seconds each melting, annealing and extension at 98° C., 61° C. and 72° C., respectively. To remove the GSP annealing sites and amplify the full-length genes, the amplified products were diluted 500x into a PCR reaction containing 0.1U/μl BtsαI and the flanking primers 5′-AGCGATCTCGGTGACGATGG-3′ and 5′-CATTAACGATGCAAGTCTCGTGG-3′ and incubated at 55C for 60 minutes prior to 10 cycles of amplification with 61C annealing temperature and 35 cycles with 65C annealing temperature.

Cloning: Genes were pooled, digested with Ndel and Xhol, ligated into plasmid pKTCTET, column purified and transformed into sufficient electrocompetent NEB 10-beta cells to yield >1000x transformants per gene being cloned. The entire transformation was cultured in 500 ml LB with 100 ug/ml AMP overnight after which plasmid was purified, diluted to 1 ng/ul to minimize transforming individual cells with multiple plasmids, and transformed into the CM-deficient strain KAl2 containing plasmid pKIMP/UAUC to yield >1000× transformants per gene. The entire transformation was cultured in 500 ml LB with 100 ug/ml AMP and 30 ug/ml CAM overnight, supplemented with 16% glycerol and frozen at −80° C.

Chorismate mutase selection assay: KAl2 glycerol stocks were cultured in LB medium overnight at 30° C., diluted to OD600 of 0.045 in non-selective M9cFY and grown at 30° C. to OD600 of ˜0.2 and washed with M9c (no FY). This pre-selected culture was inoculated into LB containing 100 ug/ml AMP, grown overnight and harvested for plasmid purification to generate the “input” sample. For selection, the culture was diluted into 500 ml M9c containing 3 ng/ml doxycycline at a calculated starting OD600 of 1e-4 and grown at 30C for 24 hours. 50 ml of the culture was harvested by centrifugation, resuspended in 2 ml LB containing 100 ng/ml AMP and grown overnight and harvested for plasmid purification.

Sequencing: Plasmids purified from input and selected cultures were amplified using two rounds of PCR with KOD polymerase to add adapters and indices for Illumina sequencing. In the first round the DNA was amplified using primers that anneal to the plasmid and add from 6 to 9 Ns to help with initial focusing, as well as part of the i5 or i7 adapters. Example primers are 5′-TGACTGGAGTTCAGACGTGTGCTCTTCCGATCTNNNNNNACGACTCACTATAGGG AGAC-3′ and 5′-CACTCTTTCCCTACACGACGCTCTTCCGATCTNNNNNNTGACTAGTCATTATTAGT GG-3′. In the second round of PCR, the remaining adaptors and TruSeq indices were added. Example primers are 5′-CAAGCAGAAGACGGCATACGAGATCGAGTAATGTGACTGGAGTTCAGACGTG-3′ and 5′-AATGATACGGCGACCACCGAGATCTACACTATAGCCTACACTCTTTCCCTACACG AC-3′. For both rounds of PCR, low cycles (16 rounds) and high initial template concentration were used to minimize amplification-induced bias. The final products were gel purified, quantified using Qubit and sequenced in a MiSeq with 2×250 cycles.

Paired-end reads were joined using FLASH (ref), trimmed to the Ndel and Xhol cloning sites and translated. Only exact matches to the designed genes were counted. Finally, relative enrichment values (r.e.) were calculated.

In FIG. 12, circuitry and hardware is also shown for acquiring, storing, processing, and distributing data from the protein assay apparatus 810, the gene synthesis apparatus 820 (also referred to as gene synthesis apparatus/system 820), and the gene expression apparatus 830. The circuitry and hardware include: a processor 870, a network controller 874, a memory 878, and a data acquisition system (DAS) 876. The protein optimization system 800 can include a data channel (not shown) that routes detection measurement results from the respective apparatuses (e.g., the protein assay apparatus 810, the gene synthesis apparatus 820 (also referred to as gene synthesis apparatus/system 820), and the gene expression apparatus 830) to the DAS 876, a processor 870, a memory 878, and a network controller 874. The data acquisition system 876 can control the acquisition, digitization, and routing of the detection data from various sensors and detectors. The processor 870 performs functions including training the machine-learning model 115, fitting the functionality landscape, and controlling the respective apparatuses, as discussed herein.

The processor 870 can be configured to perform various steps of methods and processes described herein. The processor 870 can include a CPU that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VHDL, Verilog, or any other hardware description language and the code may be stored in an electronic memory directly within the FPGA or CPLD, or as a separate electronic memory. Further, the memory may be non-volatile, such as ROM, EPROM, EEPROM or FLASH memory. The memory can also be volatile, such as static or dynamic RAM, and a processor, such as a microcontroller or microprocessor, may be provided to manage the electronic memory as well as the interaction between the FPGA or CPLD and the memory.

Alternatively, the CPU in the processor 870 can execute a computer program including a set of computer-readable instructions that perform various steps of method 10 and/or method 10′, the program being stored in any of the above-described non-transitory electronic memories and/or a hard disk drive, CD, DVD, FLASH drive or any other known storage media. Further, the computer-readable instructions may be provided as a utility application, background daemon, or component of an operating system, or combination thereof, executing in conjunction with a processor, such as a Xenon processor from Intel of America or an Opteron processor from AMD of America and an operating system, such as Microsoft VISTA, UNIX, Solaris, LINUX, Apple, MAC-OS and other operating systems known to those skilled in the art. Further, CPU can be implemented as multiple processors cooperatively working in parallel to perform the instructions.

The memory 878 can be a hard disk drive, CD-ROM drive, DVD drive, FLASH drive, RAM, ROM or any other electronic storage known in the art.

The network controller 874, such as an Intel Ethernet PRO network interface card from Intel Corporation of America, can interface between the various parts of the protein optimization system 800. Additionally, the network controller 874 can also interface with an external network. As can be appreciated, the external network can be a public network, such as the Internet, or a private network such as an LAN or WAN network, or any combination thereof and can also include PSTN or ISDN sub-networks. The external network can also be wired, such as an Ethernet network, or can be wireless such as a cellular network including EDGE, 3G and 4G wireless cellular systems. The wireless network can also be WiFi, Bluetooth, or any other wireless form of communication that is known.

Now a more detailed description of training an artificial neural network (e.g., a VAE) is provided (e.g., process 310). Here, the target data are, e.g., the output amino acid sequences, and the input data are the same output amino acid sequences, as described above.

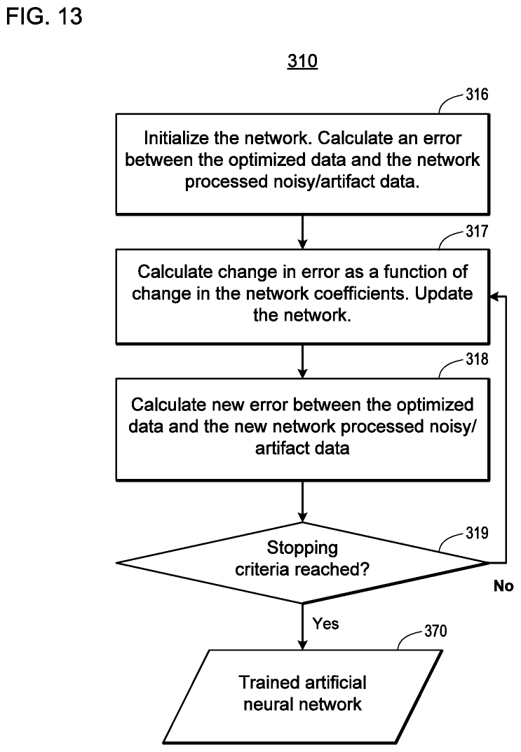

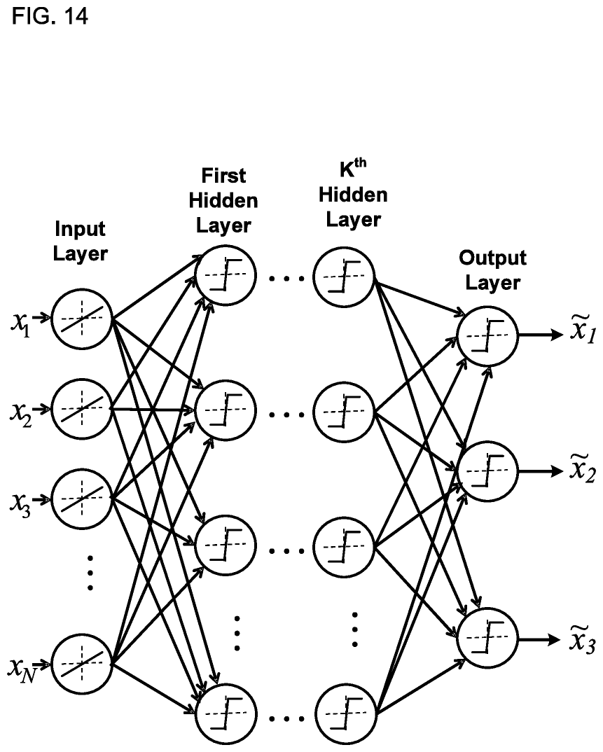

FIG. 13 shows a flow diagram of one implementation of the training process 310. In process 310, input data and target data are used as training data to train an artificial neural network, resulting in the trained artificial neural network 370 being output from step 319 of process 310. The offline DL training process 310 trains the artificial neural network using a large number of amino acid sequences of the input data to train the artificial neural network.

In process 310, a set of training data is obtained, and the network is iteratively updated to reduce the error (e.g., the value produced by a loss function). The artificial neural network infers the mapping implied by the training data, and the loss function produces an error value related to the mismatch between the target data and the result produced by applying a current incarnation of the artificial neural network to the input data. For example, in certain implementations, the loss function can use the mean-squared error to minimize the average squared error. In the case of a of multilayer perceptrons (MLP) neural network, the backpropagation algorithm can be used for training the network by minimizing the mean-squared-error-based loss function using a (stochastic) gradient descent method.

In step 316 of process 310, an initial guess is generated for the coefficients of the artificial neural network. For example, the initial guess can be based on one of a LeCun initialization, a Xavier initialization, and a Kaiming initialization.

Steps 316 through 319 of process 310 provide a non-limiting example of an optimization method for training the artificial neural network.

An error is calculated (e.g., using a loss function or a loss function) to represent a measure of the difference (e.g., a distance measure) between the target data (i.e., ground truth) and the input data after applying a current version of the network. The error can be calculated using any known loss function or distance measure, including those loss functions described above. Further, in certain implementations the error/loss function can be calculated using one or more of a hinge loss and a cross-entropy loss. In certain implementations, the loss function can be the lₚ-norm of the difference between the target data and the result of applying the input data to the artificial neural network. Different values of “p” in the lₚ-norm can be used to emphasize different aspects of the noise. In certain implementations, rather than minimizing an lₚ-norm of the difference between the target data and the result from the input data, the loss function can represent a similarity (e.g., using a peak signal-to-noise ratio (PSNR) or a structural similarity (SSIM) index).

In certain implementations, the network is trained using backpropagation. Backpropagation can be used for training neural networks and is used in conjunction with gradient descent optimization methods. During a forward pass, the algorithm computes the network's predictions based on the current parameters (θ). These predictions are then input into the loss function, by which they are compared to the corresponding ground truth labels (i.e., the high-quality target data). During the backward pass, the model computes the gradient of the loss function with respect to the current parameters, after which the parameters are updated by taking a step of size of a predefined size in the direction of minimized loss (e.g., in accelerated methods, such that the Nesterov momentum method and various adaptive methods, the step size can be selected to more quickly converge to optimize the loss function).

The optimization method by which the backprojection is performed can use one or more of gradient descent, batch gradient descent, stochastic gradient descent, and mini-batch stochastic gradient descent. The forward and backwards passes can be performed incrementally through the respective layers of the network. In the forward pass, the execution starts by feeding the inputs through the first layer, thus creating the output activations for the subsequent layer. This process is repeated until the loss function at the last layer is reached. During the backward pass, the last layer computes the gradients with respect to its own learnable parameters (if any) and also with respect to its own input, which serves as the upstream derivatives for the previous layer. This process is repeated until the input layer is reached.

Returning to FIG. 13, step 317 of process 310 determines a change in the error as a function of the change in the network can be calculated (e.g., an error gradient), and this change in the error can be used to select a direction and step size for a subsequent change to the weights/coefficients of the artificial neural network. Calculating the gradient of the error in this manner is consistent with certain implementations of a gradient descent optimization method. In certain other implementations, this step can be omitted and/or substituted with another step in accordance with another optimization algorithm (e.g., a non-gradient descent optimization algorithm like simulated annealing or a genetic algorithm), as would be understood by one of ordinary skill in the art.

In step 317 of process 310, a new set of coefficients are determined for the artificial neural network. For example, the weights/coefficients can be updated using the changed calculated in step 317, as in a gradient descent optimization method or an over-relaxation acceleration method.

In step 318 of process 310, a new error value is calculated using the updated weights/coefficients of the artificial neural network.

In step 319, predefined stopping criteria are used to determine whether the training of the network is complete. For example, the predefined stopping criteria can evaluate whether the new error and/or the total number of iterations performed exceed predefined values. For example, the stopping criteria can be satisfied if either the new error falls below a predefined threshold or if a maximum number of iterations is reached. When the stopping criteria is not satisfied, the training process performed in process 310 will continue back to the start of the iterative loop by returning and repeating step 317 using the new weights and coefficients (the iterative loop includes steps 317, 318, and 319). When the stopping criteria are satisfied, the training process performed in process 310 is completed.

FIG. 14 shows an example of the inter-connections between layers in the artificial neural network. The artificial neural network can include fully connected, convolutional, and the pooling layer, all of which are explained below. In certain preferred implementations of the artificial neural network, convolutional layers are placed close to the input layer, whereas fully connected layers, which perform the high-level reasoning, are place further down the architecture towards the loss function. Pooling layers can be inserted after convolutions and proved a reduction lowering the spatial extent of the filters, and thus the amount of learnable parameters. Activation functions are also incorporated into various layers to introduce nonlinearity and enable the network to learn complex predictive relationships. The activation function can be a saturating activation functions (e.g., a sigmoid or hyperbolic tangent activation function) or rectified activation function (e.g., the Rectified Linear Unit (ReLU) applied in the first and second examples discussed above). The layers of the artificial neural network can also incorporate batch normalization, as also exemplified in the first and second examples discussed above.

FIG. 14 shows an example of a general artificial neural network (ANN) having N inputs, K hidden layers, and three outputs. Each layer is made up of nodes (also called neurons), and each node performs a weighted sum of the inputs and compares the result of the weighted sum to a threshold to generate an output. ANNs make up a class of functions for which the members of the class are obtained by varying thresholds, connection weights, or specifics of the architecture such as the number of nodes and/or their connectivity. The nodes in an ANN can be referred to as neurons (or as neuronal nodes), and the neurons can have inter—connections between the different layers of the ANN system. The synapses (i.e., the connections between neurons) store values called “weights” (also interchangeably referred to as “coefficients” or “weighting coefficients”) that manipulate the data in the calculations. The outputs of the ANN depend on three types of parameters: (i) the interconnection pattern between the different layers of neurons, (ii) the learning process for updating the weights of the interconnections, and (iii) the activation function that converts a neuron's weighted input to its output activation.

Mathematically, a neuron's network function m(x) is defined as a composition of other functions nᵢ (x), which can further be defined as a composition of other functions. This can be conveniently represented as a network structure, with arrows depicting the dependencies between variables, as shown in FIG. 14. For example, the ANN can use a nonlinear weighted sum, wherein m(x)=K(Σᵢwᵢnᵢ (x)), where K (commonly referred to as the activation function) is some predefined function, such as the hyperbolic tangent.

In FIG. 14, the neurons (i.e., nodes) are depicted by circles around a threshold function. For the non-limiting example shown in FIG. 14, the inputs are depicted as circles around a linear function, and the arrows indicate directed connections between neurons.

While certain implementations have been described, these implementations have been presented by way of example only, and are not intended to limit the teachings of this disclosure. Indeed, the novel methods, apparatuses and systems described herein may be embodied in a variety of other forms; furthermore, various omissions, substitutions and changes in the form of the methods, apparatuses and systems described herein may be made without departing from the spirit of this disclosure.

Aspects of the Present Disclosure

The following aspects of the disclosure are exemplary only and not intended to limit the scope of the disclosure.

1. A method of designing proteins having a desired functionality, the method comprising: determining candidate amino acid sequences of synthetic proteins using a machine-learning model that has been trained to learn implicit patterns in a training dataset amino acid sequences of proteins, the machine-learning model expressing the learned implicit patterns in a trained model; performing an iterative loop, wherein each iteration of the loop comprises; synthesizing candidate genes and producing candidate proteins corresponding to the respective candidate amino acid sequences, each of the candidate genes coding for the corresponding candidate amino acid sequence; evaluating a degree to which the candidate proteins respectively exhibit a desired functionality by measuring values indicative of properties of the candidate proteins using one or more assays; and, when one or more stopping criteria of the iterative loop have not been satisfied, calculating, from the measured values, a fitness function assigned to each sequence, and selecting, using a combination of the fitness function together with the machine-learning model, new candidate amino acid sequences for a subsequent iteration.

2. The method of aspect 1, wherein the implicit patterns are learned in a latent space, and wherein determining the candidate amino acid sequences further comprises determining the latent space has a reduced dimension relative to a characteristic dimension of the amino acid sequences of the training dataset.

3. The method as in one of aspects 1-2, wherein the training dataset comprises a multiple sequence alignment of evolutionarily-related proteins, amino acid sequences in the multiple sequence alignment have a sequence length L, and a characteristic dimension of the training dataset is large enough to accommodate 20^{L} combinations of amino acids corresponding to the sequence length L.

4. The method as in one of aspects 1-3, wherein the training dataset comprises a multi-sequence alignment of evolutionarily-related proteins, and a characteristic dimension of the amino acid sequences of the training dataset is a product L×K, where L is a length of one of the amino acid sequences of the training dataset times and K is a number of possible types of amino acids.

5. The method of aspect 4, wherein the amino acids are natural amino acids and K is equal to or less than 20.

6. The method of aspect 4, wherein at least one of the possible types of amino acids is a non-natural amino acid.

7. The method as in one of aspects 1-6, wherein the training dataset comprises proteins that are related by a common function, which is at least one of (i) a common binding function, (ii) a common allosteric function, and (iii) a common catalytic function.

8. The method as in one of aspects 1-7, wherein the training dataset used to train the machine-learning model comprises proteins that are related by at least one of (i) a common ancestor, (ii) a common three-dimensional structure, (iii) a common function, (iv) a common domain structure, and (ν) a common evolutionary selection pressure.

9. The method as in one of aspects 1-8, wherein the step of performing the iterative loop further comprises updating, when one or more stopping criteria have not been satisfied, the machine-learning model based on an updated training dataset of proteins that includes amino acid sequences of the candidate proteins, and selecting the new candidate amino acid sequences for the subsequent iteration using the combination of the fitness function together with the machine-learning model after having been updated based on the updated training dataset.

10. The method as in one of aspects 1-x, wherein the machine-learning model is one of (i) a variational auto-encoder (VAE) network, (ii) a restricted Boltzmann machine (RBM) network, (iii) a direct coupling analysis (DCA) model, (iv) a statistical coupling analysis (SCA) model, and (ν) a generative adversarial network (GAN).

11. The method of aspect 2, wherein the machine-learning model is a network model that performs encoding and decoding/generation, the encoding being performed by mapping an input amino acid sequence to a point in the latent space, and the decoding/generation being performed by mapping the point in the latent space to an output amino acid sequence, and the machine-learning model is trained to optimize an objective function, a component of which represents a degree to which the input amino acid sequence and the output amino acid sequence match, such that, when trained using the training dataset, the machine-learning model generates output amino acid sequences that approximately match the amino acid sequences of the training dataset that are applied as inputs to the machine-learning model.

12. The method as in one of aspects 1-x, wherein the machine-learning model is an unsupervised statistics-based model that learns design rules based on first-order statistics and second order statistics of the amino acid sequences of the training dataset, and the machine-learning model is a generative model trained by a machine-learning method to generate output amino acid sequence that are consistent with the learned design rules.

13. The method as in one of aspects 1-12, further comprising training the machine-learning model using the training dataset to learn external fields and residue-residue couplings of a Potts model to generate a DCA model of the training dataset, the DCA model being used as the machine-learning model.

14. The method of aspect 13, wherein the DCA model is trained using one of a Boltzmann machine learning method, a mean-field solution method, a Monte Carlo gradient descent method, and a pseudo-likelihood maximization method.

15. The method of aspect 13, wherein the step of determining the candidate amino acid sequences further includes selecting the candidate amino acid sequences from a Boltzmann statistical distribution based on a Hamiltonian of the Potts model as trained at one or more one or more predefined temperatures, the candidate amino acid sequences being selected using at least one of a Markov chain Monte Carlo (MCMC) method, a simulated annealing method, a simulated heating method, a genetic algorithm, a basin hopping method, a sampling method and an optimization method, to draw samples from the Boltzmann statistical distribution.

16. The method of aspect 15, wherein the step of selecting the new candidate amino acid sequences for the subsequent iteration further comprises biasing a selection of amino acid sequences from a Boltzmann statistical distribution based on a Hamiltonian of the trained Potts model at one or more predefined temperatures, wherein the biasing of the selection of amino acid sequences is based on the fitness function to increase a number of the amino acid sequences being selected that more closely match amino acid sequences of measured candidate proteins for which the measured values indicated that the desired functionality was greater than a mean, a median, or a mode of the measured values.

17. The method of aspect 15, wherein the step of selecting the new candidate amino acid sequences for the subsequent iteration further comprises randomly drawing amino acid sequences from a statistical distribution in which a Boltzmann statistical distribution based on a Hamiltonian of the Potts model as trained is weighted by the fitness function to increase a likelihood that the samples are drawn from regions in the latent space that are more representative of candidate amino acid sequences that exhibit more of the desired functionality than do the candidate amino acid sequences corresponding to other regions of the latent space.

18. The method as in one of aspects 1-17, further comprising training the machine-learning model using the training dataset to learn a positional coevolution matrix to generate an SCA model of the training dataset, the SCA model being used as the machine-learning model.

19. The method of aspect 18, further comprising: generating a sample set of amino acid sequences by performing simulated annealing or simulated heating using the SCA model, the sample set of amino acid sequences expressing the learned implicit patterns of the training dataset, and selecting the candidate amino acid sequences from the generated sample set of amino acid sequences.

20. The method as in one of aspects 1-19, wherein the step of selecting the new candidate amino acid sequences for the subsequent iteration further comprises performing a linear or nonlinear dimensionality reduction on the candidate amino acid sequences of the candidate proteins to rank components of a low-dimensional model, and biasing the selection of the amino acid sequences to increase a number of amino acid sequences selected in one or more neighborhoods within a space of leading components of the low-dimensional model in which amino acid sequences that correspond to measured values indicating a high degree of a desired functionality cluster.

21. The method of aspect 20, wherein the nonlinear dimensionality reduction is a principal component analysis and leading components of the low-dimensional model are the principle components of a principal component analysis represented by a set of eigenvectors that correspond to a set of largest eigenvalues of a correlation matrix.

22. The method of aspects 20, wherein the nonlinear dimensionality reduction is an independent component analysis in which the eigenvectors are subject to a rotation and scaling operation to identify functionally-independent modes of sequence variation.

23. The method of aspect 11, wherein the step of determining the candidate amino acid sequences further comprises: identifying a neighborhood within the latent space corresponding to amino acid sequences of proteins that are selected as being likely to exhibit the desired functionality, selecting points with the identified neighborhood within the latent space, and using the decoding/generation performed by the machine-learning model to map from the selected points to respective candidate amino acid sequences, which are then used as the candidate amino acid sequences.

24. The method of aspect 11, wherein the step of selecting the new candidate amino acid sequences for the subsequent iteration further comprises: identifying, based on the fitness function, regions within the latent space that or more likely than other regions to exhibit the desired functionality or are too sparsely sampled to make statistically significant estimates regarding the desired functionality, selecting points within the identified regions within the latent space, and using the decoding/generation performed by the machine-learning model to map from the selecting points to respective candidate amino acid sequences, which are then used as the new candidate amino acid sequences for the subsequent iteration further comprises.

25. The method of aspect 24, wherein: the step of identifying the regions within the latent space further includes generating a density function within the latent space based on the fitness function, and the step of selecting the points within the identified regions within the latent space further includes selecting the points to be statistically representative of the density function.

26. The method as in one of aspects 1-25, wherein the step of calculating the fitness function further comprises performing supervised learning of a functionality landscape approximating the measured values of the candidate proteins as a function of corresponding positions within the latent space, wherein the fitness function is based, at least in part, on the functionality landscape.

27. The method of aspect 26, wherein, for a given point in the latent space, the functionality landscape provides an estimated value of functionality for a corresponding amino acid sequence to the given point, and the estimated value of the functionality is at least one of (i) a statistical probability of the corresponding amino acid sequence based on the machine-learning model, (ii) a statistical energy or physical energy of folding the corresponding amino acid sequence, the statistical energy being predicted computationally based on a statistical scoring function, and (iii) an activity of the statistical energy in performing a particular structural or functional role, the activity being predicted computationally or measured experimentally.

28. The method of aspect 26, wherein the fitness function is the functionality landscape.

29. The method of aspect 26, wherein the fitness function is based on the functionality landscape and at least one other parameter selected from a sequence-similarity landscape and a stability landscape, the sequence-similarity landscape estimating a degree to which the proteins corresponding to points in the latent space are similar to a predefined ensemble of proteins, and the stability landscape estimating a degree to which the proteins corresponding to points in the latent space are stable.

30. The method of aspect 29, wherein the stability landscape is based on numerical simulations of protein folding for proteins corresponding to points in the latent space that are stable.

31. The method of aspect 29, wherein the functionality landscape and the at least one other parameter define a multi-objective optimization space, and the candidate amino acid sequences for the subsequent iteration are selected by determining a convex hull within the multi-objective optimization space as a Pareto frontier, selecting points within the latent space that lie on the Pareto frontier, and using the machine-learning model to map the selected points to amino acid sequences, which are then used as the candidate amino acid sequences for the subsequent iteration.

32. The method of aspect 29, wherein the functionality landscape is generated by performing supervised learning using either supervised classification or regression analysis, the supervised learning being one of (i) a multivariable linear, polynomial, step, lasso, ridge, kernel, or nonlinear regression method, (ii) a support vector regression (SVR) method, (iii) a Gaussian process regression (GPR) method, (iv) a decision tree (DT) method, (ν) a random forests (RF) method, and (vi) an artificial neural network (ANN).

33. The method of aspect 30, wherein the functionality landscape further includes an uncertainty value as a function of position within the latent space, the uncertainty value representing an uncertainty that has been estimated for how well the functionality landscape approximates the measured values.

34. The method of aspect 33, further comprising selecting some of the candidate amino acid sequences for the subsequent iteration to correspond with regions in the latent space having larger uncertainty values than other regions such that, in the subsequent iteration, measured values corresponding to the some of the candidate amino acid sequences will reduce the larger uncertainty values due to increased sampling in the regions of the larger uncertainty values.

35. The method as in one of aspects 1-34, wherein the step of measuring the values of the candidate proteins includes measuring the values using at least one of (i) an assay that measures growth rate as an indicia of the desired functionality, (ii) an assay that measures gene expression as an indicia of the desired functionality, and (iii) an assay that uses microfluidics and fluorescence to measure gene expression or activity as indicia of the desired functionality.

36. The method as in one of aspects 1-34, wherein the step of synthesizing the candidate genes further comprises using polymerase cycling/chain assembly (PCA) in which oligonucleotides (oligos) having overlapping extensions are provided in a solution, wherein the oligos are cycled through a series of temperatures whereby the oligos are combined into larger oligos by steps of (i) denaturing the oligos, (ii) annealing the overlapping extensions, and (iii) extending non-overlapping extensions.

37. The method as in one of aspects 1-34, wherein the step of performing the iterative loop further comprises evolving a parameter of the one or more assays that is evolved from a starting value to a final value, such that during a first iteration the candidate genes exhibit the desired functionality when measured at the starting value, but not when measured at the final value, and during a final iteration the candidate genes exhibit the desired functionality when measured at the final value.

38. The method of aspect 37, wherein the parameter is one of (i) a temperature, (ii) a pressure, (iii) a light condition, (iv) a pH value, and (ν) a concentration of a substance in a medium used for the one or more assays.

39. The method of aspect 37, wherein the parameter of the one or more assays is selected to evaluate the candidate amino acid sequences with respect to a combination of internal phenotypes and external environmental conditions.

40. The method as in one of aspects 1-39, wherein the step of performing the iterative loop further comprises, when the one or more stopping criteria of the iterative loop are satisfied, stopping the iterative loop and outputting information of one or more genetic codes corresponding to one or more of the candidate genes most exhibiting the desired functionality.

41. A system for designing proteins having a desired functionality, the system comprising: a gene synthesis system configured to synthesize genes based on input gene sequences that code for respective amino acid sequences, and generate proteins from the synthesized genes; an assay system configured to measure values of proteins received from the gene synthesis system, the measured values providing indicia of a desired functionality; and processing circuitry configured to: determine candidate amino acid sequences of synthetic proteins using a machine-learning model that has been trained to learn implicit patterns in a training dataset of amino acid sequences of proteins, the machine-learning model expressing the learned implicit patterns in a trained model, and perform an iterative loop, wherein each iteration of the loop comprises, sending, to the gene synthesis system, the candidate amino acid sequences to generate candidate proteins based on the candidate amino acid sequences, receiving, from the assay system, measure values corresponding to candidate proteins based on the candidate amino acid sequences, and, when one or more stopping criteria of the iterative loop have not been satisfied, calculating, from the measured values, a fitness function assigned to each amino acid sequence, and selecting, using a combination of the fitness function together with the machine-learning model, new candidate amino acid sequences for a subsequent iteration.

42. The system of aspect 41, wherein the machine-learning model expresses the learned implicit patterns in a latent space and the processing circuitry is further configured to determine the candidate amino acid sequences, the latent space having a reduced dimension relative to a characteristic dimension of the amino acid sequences of the training dataset.

43. The system as in one of aspects 41-42, wherein the training dataset comprises a multiple sequence alignment of homologous proteins, amino acid sequences in the multiple sequence alignment have a sequence length L, and the characteristic dimension of the training dataset is large enough to accommodate 20^{L} combinations of amino acids corresponding to the sequence length L.

44. The system of aspect 42, wherein the training dataset comprises a multi-sequence alignment of evolutionarily-related proteins, and the characteristic dimension of the amino acid sequences of the training dataset is a product L×K, where L is a length of one of the amino acid sequences of the training dataset and K is a number of possible types of amino acids.

45. The system of aspect 44, wherein the amino acids are natural amino acids and K is equal to or less than 20.

46. The system of aspect 44, wherein at least one of the types of amino is a non-natural amino acid.

47. The system as in one of aspects 41-46, wherein the training dataset comprises proteins that are related by a common function which is at least one of (i) a common binding function, (ii) a common allosteric function, and (iii) a common catalytic function.

48. The system as in one of aspects 41-47, wherein the training dataset used to train the machine-learning model comprises proteins that are related by at least one of (i) a common ancestor, (ii) a common three-dimensional structure, (iii) a common function, (iv) a common domain structure, and (ν) a common evolutionary selection pressure.

49. The system as in one of aspects 41-48, wherein the processing circuitry is further configured to performing the iterative loop, updating, when one or more stopping criteria have not been satisfied, the machine-learning model based on an updated training dataset of proteins that includes amino acid sequences of the candidate proteins, and selecting the new candidate amino acid sequences for the subsequent iteration using the combination of the fitness function together with the machine-learning model after having been updated based on the updated training dataset.

50. The system as in one of aspects 41-49, wherein the machine-learning model is one of (i) a variational auto-encoder (VAE) network, (ii) a restricted Boltzmann machine (RBM) network, (iii) a direct coupling analysis (DCA) model, (iv) a statistical coupling analysis (SCA) model, and (ν) a generative adversarial network (GAN).

51. The system of aspect 42, wherein the machine-learning model is a network model that performs encoding and decoding/generation, the encoding being performed by mapping an input amino acid sequence to a point in the latent space, and the decoding/generation being performed by mapping the point in the latent space to an output amino acid sequence, and the machine-learning model is trained to optimize an objective function, a component of which represents a degree to which the input amino acid sequence and the output amino acid sequence match, such that, when trained using the training dataset, the machine-learning model generates output amino acid sequences that approximately match the amino acid sequences of the training dataset that are applied as inputs to the machine-learning model.

52. The system as in one of aspects 41-51, wherein the machine-learning model is an unsupervised statistics-based model that learns design rules based on first-order statistics and second order statistics of the amino acid sequences of the training dataset, and the machine-learning model is a generative model trained to generate output amino acid sequence that are consistent with the learned design rules.

53. The system as in one of aspects 41-52, wherein the processing circuitry is further configured to train the machine-learning model using the training dataset to learn external fields and residue-residue couplings of a Potts model to generate a DCA model of the training dataset, the DCA model being used as the machine-learning model.

54. The system of aspect 53, wherein the DCA model is trained using one of a Boltzmann machine learning method, a mean-field solution method, a Monte Carlo gradient descent method, and a pseudo-likelihood maximization method.

55. The system of aspect 53, wherein the processing circuitry is further configured to determine the candidate amino acid sequences by selecting the candidate amino acid sequences from a Boltzmann statistical distribution based on a Hamiltonian of the Potts model as trained at one or more predefined temperatures, the candidate amino acid sequences being selected using at least one of a Markov chain Monte Carlo (MCMC) method, a simulated annealing method, a simulated heating method, a genetic algorithm, a basin hopping method, a sampling method and an optimization method, to draw samples from the Boltzmann statistical distribution.

56. The system of aspect 55, wherein the processing circuitry is further configured to select the new candidate amino acid sequences for the subsequent iteration by biasing a selection of amino acid sequences from a Boltzmann statistical distribution based on a Hamiltonian of the Potts model as trained at one or more predefined temperatures, wherein the biasing of the selection of amino acid sequences is based on the fitness function to increase a number of the amino acid sequences being selected that more closely match amino acid sequences of measured candidate proteins for which the measured values indicated that the desired functionality was greater than a mean, a median, or a mode of the measured values.

57. The system of aspect 53, wherein the processing circuitry is further configured to select the new candidate amino acid sequences for the subsequent iteration by randomly drawing amino acid sequences from a statistical distribution in which a Boltzmann statistical distribution based on a Hamiltonian of the trained Potts model is weighted by the fitness function to increase a likelihood that the samples are drawn from regions in the latent space that are more representative of candidate amino acid sequences that exhibit more of the desired functionality than do the candidate amino acid sequences corresponding to other regions of the latent space.

58. The system as in one of aspects 41-57, wherein the processing circuitry is further configured to train the machine-learning model using the training dataset to learn a positional coevolution matrix to generate an SCA model of the training dataset, the SCA model being used as the machine-learning model.

59. The system of aspect 58, wherein the processing circuitry is further configured to generate a sample set of amino acid sequences by performing simulated annealing or simulated heating using the SCA model, the sample set of amino acid sequences expressing the learned implicit patterns of the training dataset, and wherein the processing circuitry is further configured to select the candidate amino acid sequences from the sample set of amino acid sequences.

60. The system as in one of aspects 41-59, wherein the processing circuitry is further configured to select the new candidate amino acid sequences for the subsequent iteration by performing a linear or nonlinear dimensionality reduction on the candidate amino acid sequences of the measured candidate proteins to rank components of a low-dimensional model, and biasing the selection of the amino acid sequences to increase a number of amino acid sequences selected in one or more neighborhoods within a space of leading components of the low-dimensional model in which amino acid sequences that correspond to measured values indicating a high degree of a desired functionality cluster.

61. The system of aspect 60, wherein the nonlinear dimensionality reduction is a principal component analysis or independent component analysis, and leading components of the low-dimensional model are principle components of the principal component analysis or the independent components of the independent component analysis.

62. The system of aspect 51, wherein the processing circuitry is further configured to determine the candidate amino acid sequences by identifying a neighborhood within the latent space corresponding to amino acid sequences of proteins that are selected as being likely to exhibit the desired functionality, selecting points with the identified neighborhood within the latent space, and using the decoding/generation performed by the machine-learning model to map from the selected points to respective candidate amino acid sequences, which are then used as the candidate amino acid sequences.

63. The system of aspect 51, wherein the processing circuitry is further configured to select the new candidate amino acid sequences for the subsequent iteration by identifying, based on the fitness function, regions within the latent space that are more likely than other regions to exhibit the desired functionality or are too sparsely sampled to make statistically significant estimates regarding the desired functionality, selecting points within the identified regions within the latent space, and using the decoding/generation performed by the machine-learning model to map from the selecting points to respective candidate amino acid sequences, which are then used as the new candidate amino acid sequences for the subsequent iteration.

64. The system of aspect 63, wherein the processing circuitry is further configured to: identify the regions within the latent space by generating a density function within the latent space based on the fitness function, and select the points within the identified regions within the latent space by selecting the points to be statistically representative of the density function.

65. The system as in one of aspects 41-64, wherein the processing circuitry is further configured to, in calculating the fitness function, perform supervised learning of a functionality landscape approximating the measured values of the candidate proteins as a function of corresponding positions within the latent space, wherein the fitness function is based, at least in part, on the functionality landscape.

66. The system of aspect 65, wherein, for a given point on the latent space, the functionality landscape provides an estimated value of functionality for a corresponding amino acid sequence to the given point, and the estimated value of the functionality is at least one of (i) a statistical probability of the corresponding amino acid sequence based on the machine-learning model, (ii) a statistical energy or a physical energy of folding the corresponding amino acid sequence, the statistical energy being predicted computationally based on a statistical scoring function, and (iii) an activity of the statistical energy in performing a particular structural or functional role, the activity being predicted computationally or measured experimentally.

67. The system of aspect 65, wherein the fitness function is the functionality landscape.

68. The system of aspect 65, wherein the fitness function is based on the functionality landscape and at least one other parameter selected from a sequence-similarity landscape and a stability landscape, the sequence-similarity landscape estimating a degree to which the proteins corresponding to points in the latent space are similar to a predefined ensemble of proteins, and the stability landscape estimating a degree to which the proteins corresponding to points in the latent space are stable.

69. The system of aspect 68, wherein the stability landscape is based on numerical simulations of protein folding for proteins corresponding to points in the latent space that are stable.

70. The system of aspect 69, wherein the functionality landscape and the at least one other parameter define a multi-objective optimization space, and the new candidate amino acid sequences for the subsequent iteration are selected by determining a convex hull within the multi-objective optimization space as a Pareto frontier, selecting points within the latent space that lie on the Pareto frontier, and using the machine-learning model to map the selected points to amino acid sequences, which are then used as the new candidate amino acid sequences for the subsequent iteration.

71. The system of aspect 65, wherein the functionality landscape is generated by performing supervised learning using either supervised classification or regression analysis, the supervised learning being one of (i) a multivariable linear, polynomial, step, lasso, ridge, kernel, or nonlinear regression method, (ii) a support vector regression (SVR) method, (iii) a Gaussian process regression (GPR) method, (iv) a decision tree (DT) method, (ν) a random forests (RF) method, and (vi) an artificial neural network (ANN).

72. The system of aspect 65, wherein the functionality landscape further includes an uncertainty value as a function of position within the latent space, the uncertainty value representing an uncertainty that has been estimated for how well the functionality landscape approximates the measured values.

73. The system as in one of aspects 41-72, wherein the processing circuitry is further configured to select some of the new candidate amino acid sequences for the subsequent iteration to correspond with regions in the latent space having larger uncertainty values than other regions such that, in the subsequent iteration, measured values corresponding to the some of the candidate amino acid sequences will reduce the larger uncertainty values due to increased sampling in the regions of the larger uncertainty values.

74. The system as in one of aspects 41-73, wherein the assay system is further configured to measure the values of the candidate proteins using at least one of (i) an assay that measures growth rate as an indicia of the desired functionality, (ii) an assay that measures gene expression as an indicia of the desired functionality, and (iii) an assay that uses microfluidics and fluorescence to measure gene expression or activity as indicia of the desired functionality.

75. The system as in one of aspects 41-74, wherein the gene synthesis system is further configured to synthesize the candidate genes using polymerase cycling/chain assembly (PCA) in which oligonucleotides (oligos) having overlapping extensions are provided in a solution, wherein the oligos are cycled through a series of temperatures whereby the oligos are combined into larger oligos by steps of (i) denaturing the oligos, (ii) annealing the overlapping extensions, and (iii) extending non-overlapping extensions.

76. The system as in one of aspects 41-x, wherein the processing circuitry is further configured to perform the iterative loop such that a parameter of the one or more assays is evolved from a starting value to a final value, such that during a first iteration the candidate genes exhibit the desired functionality when measured at the starting value, but not when measured at the final value, and during a final iteration the candidate genes exhibit the desired functionality when measured at the final value.

77. The system of aspect 76, wherein the parameter of the one or more assays is one of (i) a temperature, (ii) a pressure, (iii) a light condition, (iv) a pH value, and (ν) a concentration of a substance in a medium used for the one or more assays.

78. The system of aspect 76, wherein the parameter of the one or more assays is to evaluate the candidate amino acid sequences with respect to a combination of internal phenotypes and external environmental conditions.

79. A non-transitory computer readable storage medium including executable instructions, wherein the instructions, when executed by circuitry, cause the circuitry to perform a method comprising steps of: determining candidate amino acid sequences of synthetic proteins using a machine-learning model that has been trained to learn implicit patterns in a training dataset of proteins, the machine-learning model expressing the learned implicit patterns, and performing an iterative loop, wherein each iteration of the loop comprises: determining candidate genetic sequences based on the candidate amino acid sequences, sending to a gene synthesis system, the candidate genetic sequences to be synthesized into candidate genes to produce candidate proteins, receiving from an assay system, measured values generated by measuring the candidate proteins using one or more assays, and when one or more stopping criteria of the iterative loop have not been satisfied, calculating a fitness function from the measured values, and selecting, using a combination of the fitness function together with the machine-learning model, additional candidate amino acid sequences for a subsequent iteration.

80. A method of designing sequence-defined molecules having a desired functionality, the method comprising: determining candidate sequences of molecules, which are sequence defined, the candidate sequences being generated using a machine-learning model that has been trained to learn implicit patterns in a training dataset of sequence-defined molecules, the machine-learning model expressing the learned implicit patterns; and performing an iterative loop, wherein each iteration of the loop comprises: synthesizing candidate sequences corresponding to the candidate molecules, evaluating a degree to which the candidate molecules respectively exhibit a desired functionality by measuring values of the candidate molecules using one or more assays, and when one or more stopping criteria of the iterative loop have not been satisfied, calculating a fitness function from the measured values, and selecting, using a combination of the fitness function together with the machine-learning model, additional candidate sequences for a subsequent iteration.

81. The method of aspect 80, wherein the step of performing the iterative loop further comprises updating, when one or more stopping criteria have not been satisfied, the machine-learning model based on an updated training dataset of molecules that includes sequences of the candidate molecules, and selecting the additional candidate sequences for the subsequent iteration using a combination of the fitness function together with the machine-learning model after having been updated based on the updated training dataset.

82. The method of aspect 80, wherein the molecule is a DNA molecule and the sequences are sequences of nucleotides.

83. The method of aspect 80, wherein the molecule is a RNA molecule and the sequences are sequences of nucleotides.

84. The method of aspect 80, wherein the molecule is a polymer and the sequences are sequences of chemical monomers.

85. The method as in one of aspects 1-40, wherein the candidate proteins comprise one or more of an antibody, an enzyme, a hormone, a cytokine, growth factor, clotting factor, anticoagulation factor, albumin, antigen, an adjuvant, a transcription factor, or a cellular receptor.

86. The method as in one of aspects 1-40, wherein the candidate proteins are provided for selected binding to one or more other molecules.

87. The method as in one of aspects 1-40, wherein the candidate proteins are provided to catalyze one or more chemical reactions.

88. The method as in one of aspects 1-40, wherein the candidate proteins are provided for long-range signaling.

89. The method as in one of aspects 1-40 further comprising generating or manufacturing an end product based on the candidate proteins.

90. The method as in one of aspects 1-40, wherein one or more cells are produced from the candidate proteins.

91. The method of aspect 90, wherein the cells produced from the candidate proteins are directed to or placed in one or more bins.

92. The method as in one of aspects 1-40, wherein the candidate proteins are determined by high-throughput functional screening.

93. The method of aspect 92, wherein the high-throughput functional screening is implemented by a micro-fluidics apparatus that measures the fluorescence of cells corresponding to the candidate proteins.

Additional Considerations

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules may provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and may operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112(ƒ) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed is:

1. A method of designing proteins having a desired functionality, the method comprising:
determining candidate amino acid sequences of synthetic proteins using a machine-learning model that has been trained to learn implicit patterns in a training dataset amino acid sequences of proteins, the machine-learning model expressing the learned implicit patterns in a trained model;
performing an iterative loop, wherein each iteration of the loop comprises;
synthesizing candidate genes and producing candidate proteins corresponding to the respective candidate amino acid sequences, each of the candidate genes coding for the corresponding candidate amino acid sequence;
evaluating a degree to which the candidate proteins respectively exhibit a desired functionality by measuring values indicative of properties of the candidate proteins using one or more assays; and,
when one or more stopping criteria of the iterative loop have not been satisfied, calculating, from the measured values, a fitness function assigned to each sequence, and selecting, using a combination of the fitness function together with the machine-learning model, new candidate amino acid sequences for a subsequent iteration.

2. The method of claim 1, wherein the implicit patterns are learned in a latent space, and wherein determining the candidate amino acid sequences further comprises determining the latent space has a reduced dimension relative to a characteristic dimension of the amino acid sequences of the training dataset.

3. The method of claim 1, wherein the training dataset comprises a multiple sequence alignment of evolutionarily-related proteins, amino acid sequences in the multiple sequence alignment have a sequence length L, and a characteristic dimension of the training dataset is large enough to accommodate 20^{L} combinations of amino acids corresponding to the sequence length L.

4. The method of claim 1, wherein the training dataset comprises a multi-sequence alignment of evolutionarily-related proteins, and a characteristic dimension of the amino acid sequences of the training dataset is a product L×K, where L is a length of one of the amino acid sequences of the training dataset times and K is a number of possible types of amino acids.

5. The method of claim 4, wherein the amino acids are natural amino acids and K is equal to or less than 20.

6. The method of claim 4, wherein at least one of the possible types of amino acids is a non-natural amino acid.

7. The method of claim 1, wherein the training dataset comprises proteins that are related by a common function, which is at least one of (i) a common binding function, (ii) a common allosteric function, and (iii) a common catalytic function.

8. The method of claim 1, wherein the training dataset used to train the machine-learning model comprises proteins that are related by at least one of (i) a common ancestor, (ii) a common three-dimensional structure, (iii) a common function, (iv) a common domain structure, and (ν) a common evolutionary selection pressure.

9. The method of claim 1, wherein the step of performing the iterative loop further comprises updating, when one or more stopping criteria have not been satisfied, the machine-learning model based on an updated training dataset of proteins that includes amino acid sequences of the candidate proteins, and selecting the new candidate amino acid sequences for the subsequent iteration using the combination of the fitness function together with the machine-learning model after having been updated based on the updated training dataset.

10. The method of claim 1, wherein the machine-learning model is one of (i) a variational auto-encoder (VAE) network, (ii) a restricted Boltzmann machine (RBM) network, (iii) a direct coupling analysis (DCA) model, (iv) a statistical coupling analysis (SCA) model, and (ν) a generative adversarial network (GAN).

11. The method of claim 2, wherein
the machine-learning model is a network model that performs encoding and decoding/generation, the encoding being performed by mapping an input amino acid sequence to a point in the latent space, and the decoding/generation being performed by mapping the point in the latent space to an output amino acid sequence, and the machine-learning model is trained to optimize an objective function, a component of which represents a degree to which the input amino acid sequence and the output amino acid sequence match, such that, when trained using the training dataset, the machine-learning model generates output amino acid sequences that approximately match the amino acid sequences of the training dataset that are applied as inputs to the machine-learning model.

12. The method of claim 1, wherein
the machine-learning model is an unsupervised statistics-based model that learns design rules based on first-order statistics and second order statistics of the amino acid sequences of the training dataset, and
the machine-learning model is a generative model trained by a machine-learning method to generate output amino acid sequence that are consistent with the learned design rules.

13. The method of claim 1, further comprising training the machine-learning model using the training dataset to learn external fields and residue-residue couplings of a Potts model to generate a DCA model of the training dataset, the DCA model being used as the machine-learning model.

14. The method of claim 13, wherein the DCA model is trained using one of a Boltzmann machine learning method, a mean-field solution method, a Monte Carlo gradient descent method, and a pseudo-likelihood maximization method.

15. The method of claim 13, wherein the step of determining the candidate amino acid sequences further includes selecting the candidate amino acid sequences from a Boltzmann statistical distribution based on a Hamiltonian of the Potts model as trained at one or more one or more predefined temperatures, the candidate amino acid sequences being selected using at least one of a Markov chain Monte Carlo (MCMC) method, a simulated annealing method, a simulated heating method, a genetic algorithm, a basin hopping method, a sampling method and an optimization method, to draw samples from the Boltzmann statistical distribution.

16. The method of claim 15, wherein the step of selecting the new candidate amino acid sequences for the subsequent iteration further comprises biasing a selection of amino acid sequences from a Boltzmann statistical distribution based on a Hamiltonian of the trained Potts model at one or more predefined temperatures, wherein the biasing of the selection of amino acid sequences is based on the fitness function to increase a number of the amino acid sequences being selected that more closely match amino acid sequences of measured candidate proteins for which the measured values indicated that the desired functionality was greater than a mean, a median, or a mode of the measured values.

17. The method of claim 15, wherein the step of selecting the new candidate amino acid sequences for the subsequent iteration further comprises randomly drawing amino acid sequences from a statistical distribution in which a Boltzmann statistical distribution based on a Hamiltonian of the Potts model as trained is weighted by the fitness function to increase a likelihood that the samples are drawn from regions in the latent space that are more representative of candidate amino acid sequences that exhibit more of the desired functionality than do the candidate amino acid sequences corresponding to other regions of the latent space.

18. The method of claim 1, further comprising training the machine-learning model using the training dataset to learn a positional coevolution matrix to generate an SCA model of the training dataset, the SCA model being used as the machine-learning model.

19. The method of claim 18, further comprising:
generating a sample set of amino acid sequences by performing simulated annealing or simulated heating using the SCA model, the sample set of amino acid sequences expressing the learned implicit patterns of the training dataset, and
selecting the candidate amino acid sequences from the generated sample set of amino acid sequences.

20. The method of claim 1, wherein the step of selecting the new candidate amino acid sequences for the subsequent iteration further comprises performing a linear or nonlinear dimensionality reduction on the candidate amino acid sequences of the candidate proteins to rank components of a low-dimensional model, and biasing the selection of the amino acid sequences to increase a number of amino acid sequences selected in one or more neighborhoods within a space of leading components of the low-dimensional model in which amino acid sequences that correspond to measured values indicating a high degree of a desired functionality cluster.

21. The method of claim 20, wherein the nonlinear dimensionality reduction is a principal component analysis and leading components of the low-dimensional model are the principle components of a principal component analysis represented by a set of eigenvectors that correspond to a set of largest eigenvalues of a correlation matrix.

22. The method of claim 20, wherein the nonlinear dimensionality reduction is an independent component analysis in which the eigenvectors are subject to a rotation and scaling operation to identify functionally-independent modes of sequence variation.

23. The method of claim 11, wherein the step of determining the candidate amino acid sequences further comprises:
identifying a neighborhood within the latent space corresponding to amino acid sequences of proteins that are selected as being likely to exhibit the desired functionality, selecting points with the identified neighborhood within the latent space, and using the decoding/generation performed by the machine-learning model to map from the selected points to respective candidate amino acid sequences, which are then used as the candidate amino acid sequences.

24. The method of claim 11, wherein the step of selecting the new candidate amino acid sequences for the subsequent iteration further comprises:
identifying, based on the fitness function, regions within the latent space that or more likely than other regions to exhibit the desired functionality or are too sparsely sampled to make statistically significant estimates regarding the desired functionality,
selecting points within the identified regions within the latent space, and
using the decoding/generation performed by the machine-learning model to map from the selecting points to respective candidate amino acid sequences, which are then used as the new candidate amino acid sequences for the subsequent iteration further comprises.

25. The method of claim 24, wherein:
the step of identifying the regions within the latent space further includes generating a density function within the latent space based on the fitness function, and
the step of selecting the points within the identified regions within the latent space further includes selecting the points to be statistically representative of the density function.

26. The method of claim 1, wherein the step of calculating the fitness function further comprises performing supervised learning of a functionality landscape approximating the measured values of the candidate proteins as a function of corresponding positions within the latent space, wherein the fitness function is based, at least in part, on the functionality landscape.

27. The method of claim 26, wherein, for a given point in the latent space, the functionality landscape provides an estimated value of functionality for a corresponding amino acid sequence to the given point, and the estimated value of the functionality is at least one of (i) a statistical probability of the corresponding amino acid sequence based on the machine-learning model, (ii) a statistical energy or physical energy of folding the corresponding amino acid sequence, the statistical energy being predicted computationally based on a statistical scoring function, and (iii) an activity of the statistical energy in performing a particular structural or functional role, the activity being predicted computationally or measured experimentally.

28. The method of claim 26, wherein the fitness function is the functionality landscape.

29. The method of claim 26, wherein the fitness function is based on the functionality landscape and at least one other parameter selected from a sequence-similarity landscape and a stability landscape, the sequence-similarity landscape estimating a degree to which the proteins corresponding to points in the latent space are similar to a predefined ensemble of proteins, and the stability landscape estimating a degree to which the proteins corresponding to points in the latent space are stable.

30. The method of claim 29, wherein the stability landscape is based on numerical simulations of protein folding for proteins corresponding to points in the latent space that are stable.

31. The method of claim 29, wherein the functionality landscape and the at least one other parameter define a multi-objective optimization space, and the candidate amino acid sequences for the subsequent iteration are selected by determining a convex hull within the multi-objective optimization space as a Pareto frontier, selecting points within the latent space that lie on the Pareto frontier, and using the machine-learning model to map the selected points to amino acid sequences, which are then used as the candidate amino acid sequences for the subsequent iteration.

32. The method of claim 29, wherein the functionality landscape is generated by performing supervised learning using either supervised classification or regression analysis, the supervised learning being one of (i) a multivariable linear, polynomial, step, lasso, ridge, kernel, or nonlinear regression method, (ii) a support vector regression (SVR) method, (iii) a Gaussian process regression (GPR) method, (iv) a decision tree (DT) method, (ν) a random forests (RF) method, and (vi) an artificial neural network (ANN).

33. The method of claim 30, wherein the functionality landscape further includes an uncertainty value as a function of position within the latent space, the uncertainty value representing an uncertainty that has been estimated for how well the functionality landscape approximates the measured values.

34. The method of claim 33, further comprising selecting some of the candidate amino acid sequences for the subsequent iteration to correspond with regions in the latent space having larger uncertainty values than other regions such that, in the subsequent iteration, measured values corresponding to the some of the candidate amino acid sequences will reduce the larger uncertainty values due to increased sampling in the regions of the larger uncertainty values.

35. The method of claim 1, wherein the step of measuring the values of the candidate proteins includes measuring the values using at least one of (i) an assay that measures growth rate as an indicia of the desired functionality, (ii) an assay that measures gene expression as an indicia of the desired functionality, and (iii) an assay that uses microfluidics and fluorescence to measure gene expression or activity as indicia of the desired functionality.

36. The method of claim 1, wherein the step of synthesizing the candidate genes further comprises using polymerase cycling/chain assembly (PCA) in which oligonucleotides (oligos) having overlapping extensions are provided in a solution, wherein the oligos are cycled through a series of temperatures whereby the oligos are combined into larger oligos by steps of (i) denaturing the oligos, (ii) annealing the overlapping extensions, and (iii) extending non-overlapping extensions.

37. The method of claim 1, wherein the step of performing the iterative loop further comprises evolving a parameter of the one or more assays that is evolved from a starting value to a final value, such that during a first iteration the candidate genes exhibit the desired functionality when measured at the starting value, but not when measured at the final value, and during a final iteration the candidate genes exhibit the desired functionality when measured at the final value.

38. The method of claim 37, wherein the parameter is one of (i) a temperature, (ii) a pressure, (iii) a light condition, (iv) a pH value, and (ν) a concentration of a substance in a medium used for the one or more assays.

39. The method of claim 37, wherein the parameter of the one or more assays is selected to evaluate the candidate amino acid sequences with respect to a combination of internal phenotypes and external environmental conditions.

40. The method of claim 1, wherein the step of performing the iterative loop further comprises, when the one or more stopping criteria of the iterative loop are satisfied, stopping the iterative loop and outputting information of one or more genetic codes corresponding to one or more of the candidate genes most exhibiting the desired functionality.

41. A system for designing proteins having a desired functionality, the system comprising:
a gene synthesis system configured to synthesize genes based on input gene sequences that code for respective amino acid sequences, and generate proteins from the synthesized genes;
an assay system configured to measure values of proteins received from the gene synthesis system, the measured values providing indicia of a desired functionality; and
processing circuitry configured to determine candidate amino acid sequences of synthetic proteins using a machine-learning model that has been trained to learn implicit patterns in a training dataset of amino acid sequences of proteins, the machine-learning model expressing the learned implicit patterns in a trained model, and
perform an iterative loop, wherein each iteration of the loop comprises,
sending, to the gene synthesis system, the candidate amino acid sequences to generate candidate proteins based on the candidate amino acid sequences,
receiving, from the assay system, measure values corresponding to candidate proteins based on the candidate amino acid sequences, and,
when one or more stopping criteria of the iterative loop have not been satisfied, calculating, from the measured values, a fitness function assigned to each amino acid sequence, and selecting, using a combination of the fitness function together with the machine-learning model, new candidate amino acid sequences for a subsequent iteration.

42. The system of claim 41, wherein the machine-learning model expresses the learned implicit patterns in a latent space and the processing circuitry is further configured to determine the candidate amino acid sequences, the latent space having a reduced dimension relative to a characteristic dimension of the amino acid sequences of the training dataset.

43. The system of claim 42, wherein the training dataset comprises a multiple sequence alignment of homologous proteins, amino acid sequences in the multiple sequence alignment have a sequence length L, and the characteristic dimension of the training dataset is large enough to accommodate 20^{L} combinations of amino acids corresponding to the sequence length L.

44. The system of claim 42, wherein the training dataset comprises a multi-sequence alignment of evolutionarily-related proteins, and the characteristic dimension of the amino acid sequences of the training dataset is a product L×K, where L is a length of one of the amino acid sequences of the training dataset and K is a number of possible types of amino acids.

45. The system of claim 44, wherein the amino acids are natural amino acids and K is equal to or less than 20.

46. The system of claim 44, wherein at least one of the types of amino is a non-natural amino acid.

47. The system of claim 41, wherein the training dataset comprises proteins that are related by a common function which is at least one of (i) a common binding function, (ii) a common allosteric function, and (iii) a common catalytic function.

48. The system of claim 41, wherein the training dataset used to train the machine-learning model comprises proteins that are related by at least one of (i) a common ancestor, (ii) a common three-dimensional structure, (iii) a common function, (iv) a common domain structure, and (ν) a common evolutionary selection pressure.

49. The system of claim 41, wherein the processing circuitry is further configured to performing the iterative loop, updating, when one or more stopping criteria have not been satisfied, the machine-learning model based on an updated training dataset of proteins that includes amino acid sequences of the candidate proteins, and selecting the new candidate amino acid sequences for the subsequent iteration using the combination of the fitness function together with the machine-learning model after having been updated based on the updated training dataset.

50. The system of claim 41, wherein the machine-learning model is one of (i) a variational auto-encoder (VAE) network, (ii) a restricted Boltzmann machine (RBM) network, (iii) a direct coupling analysis (DCA) model, (iv) a statistical coupling analysis (SCA) model, and (ν) a generative adversarial network (GAN).

51. The system of claim 42, wherein
the machine-learning model is a network model that performs encoding and decoding/generation, the encoding being performed by mapping an input amino acid sequence to a point in the latent space, and the decoding/generation being performed by mapping the point in the latent space to an output amino acid sequence, and
the machine-learning model is trained to optimize an objective function, a component of which represents a degree to which the input amino acid sequence and the output amino acid sequence match, such that, when trained using the training dataset, the machine-learning model generates output amino acid sequences that approximately match the amino acid sequences of the training dataset that are applied as inputs to the machine-learning model.

52. The system of claim 41, wherein
the machine-learning model is an unsupervised statistics-based model that learns design rules based on first-order statistics and second order statistics of the amino acid sequences of the training dataset, and
the machine-learning model is a generative model trained to generate output amino acid sequence that are consistent with the learned design rules.

53. The system of claim 41, wherein the processing circuitry is further configured to train the machine-learning model using the training dataset to learn external fields and residue-residue couplings of a Potts model to generate a DCA model of the training dataset, the DCA model being used as the machine-learning model.

54. The system of claim 53, wherein the DCA model is trained using one of a Boltzmann machine learning method, a mean-field solution method, a Monte Carlo gradient descent method, and a pseudo-likelihood maximization method.

55. The system of claim 53, wherein the processing circuitry is further configured to determine the candidate amino acid sequences by selecting the candidate amino acid sequences from a Boltzmann statistical distribution based on a Hamiltonian of the Potts model as trained at one or more predefined temperatures, the candidate amino acid sequences being selected using at least one of a Markov chain Monte Carlo (MCMC) method, a simulated annealing method, a simulated heating method, a genetic algorithm, a basin hopping method, a sampling method and an optimization method, to draw samples from the Boltzmann statistical distribution.

56. The system of claim 55, wherein the processing circuitry is further configured to select the new candidate amino acid sequences for the subsequent iteration by biasing a selection of amino acid sequences from a Boltzmann statistical distribution based on a Hamiltonian of the Potts model as trained at one or more predefined temperatures, wherein the biasing of the selection of amino acid sequences is based on the fitness function to increase a number of the amino acid sequences being selected that more closely match amino acid sequences of measured candidate proteins for which the measured values indicated that the desired functionality was greater than a mean, a median, or a mode of the measured values.

57. The system of claim 53, wherein the processing circuitry is further configured to select the new candidate amino acid sequences for the subsequent iteration by randomly drawing amino acid sequences from a statistical distribution in which a Boltzmann statistical distribution based on a Hamiltonian of the trained Potts model is weighted by the fitness function to increase a likelihood that the samples are drawn from regions in the latent space that are more representative of candidate amino acid sequences that exhibit more of the desired functionality than do the candidate amino acid sequences corresponding to other regions of the latent space.

58. The system of claim 41, wherein the processing circuitry is further configured to train the machine-learning model using the training dataset to learn a positional coevolution matrix to generate an SCA model of the training dataset, the SCA model being used as the machine-learning model.

59. The system of claim 58, wherein the processing circuitry is further configured to generate a sample set of amino acid sequences by performing simulated annealing or simulated heating using the SCA model, the sample set of amino acid sequences expressing the learned implicit patterns of the training dataset, and wherein the processing circuitry is further configured to select the candidate amino acid sequences from the sample set of amino acid sequences.

60. The system of claim 41, wherein the processing circuitry is further configured to select the new candidate amino acid sequences for the subsequent iteration by performing a linear or nonlinear dimensionality reduction on the candidate amino acid sequences of the measured candidate proteins to rank components of a low-dimensional model, and biasing the selection of the amino acid sequences to increase a number of amino acid sequences selected in one or more neighborhoods within a space of leading components of the low-dimensional model in which amino acid sequences that correspond to measured values indicating a high degree of a desired functionality cluster.

61. The system of claim 60, wherein the nonlinear dimensionality reduction is a principal component analysis or independent component analysis, and leading components of the low-dimensional model are principle components of the principal component analysis or the independent components of the independent component analysis.

62. The system of claim 51, wherein the processing circuitry is further configured to determine the candidate amino acid sequences by identifying a neighborhood within the latent space corresponding to amino acid sequences of proteins that are selected as being likely to exhibit the desired functionality, selecting points with the identified neighborhood within the latent space, and using the decoding/generation performed by the machine-learning model to map from the selected points to respective candidate amino acid sequences, which are then used as the candidate amino acid sequences.

63. The system of claim 51, wherein the processing circuitry is further configured to select the new candidate amino acid sequences for the subsequent iteration by identifying, based on the fitness function, regions within the latent space that are more likely than other regions to exhibit the desired functionality or are too sparsely sampled to make statistically significant estimates regarding the desired functionality, selecting points within the identified regions within the latent space, and using the decoding/generation performed by the machine-learning model to map from the selecting points to respective candidate amino acid sequences, which are then used as the new candidate amino acid sequences for the subsequent iteration.

64. The system of claim 63, wherein the processing circuitry is further configured to:
identify the regions within the latent space by generating a density function within the latent space based on the fitness function, and
select the points within the identified regions within the latent space by selecting the points to be statistically representative of the density function.

65. The system of claim 41, wherein the processing circuitry is further configured to, in calculating the fitness function, perform supervised learning of a functionality landscape approximating the measured values of the candidate proteins as a function of corresponding positions within the latent space, wherein the fitness function is based, at least in part, on the functionality landscape.

66. The system of claim 65, wherein, for a given point on the latent space, the functionality landscape provides an estimated value of functionality for a corresponding amino acid sequence to the given point, and the estimated value of the functionality is at least one of (i) a statistical probability of the corresponding amino acid sequence based on the machine-learning model, (ii) a statistical energy or a physical energy of folding the corresponding amino acid sequence, the statistical energy being predicted computationally based on a statistical scoring function, and (iii) an activity of the statistical energy in performing a particular structural or functional role, the activity being predicted computationally or measured experimentally.

67. The system of claim 65, wherein the fitness function is the functionality landscape.

68. The system of claim 65, wherein the fitness function is based on the functionality landscape and at least one other parameter selected from a sequence-similarity landscape and a stability landscape, the sequence-similarity landscape estimating a degree to which the proteins corresponding to points in the latent space are similar to a predefined ensemble of proteins, and the stability landscape estimating a degree to which the proteins corresponding to points in the latent space are stable.

69. The system of claim 68, wherein the stability landscape is based on numerical simulations of protein folding for proteins corresponding to points in the latent space that are stable.

70. The system of claim 69, wherein the functionality landscape and the at least one other parameter define a multi-objective optimization space, and the new candidate amino acid sequences for the subsequent iteration are selected by determining a convex hull within the multi-objective optimization space as a Pareto frontier, selecting points within the latent space that lie on the Pareto frontier, and using the machine-learning model to map the selected points to amino acid sequences, which are then used as the new candidate amino acid sequences for the subsequent iteration.

71. The system of claim 65, wherein the functionality landscape is generated by performing supervised learning using either supervised classification or regression analysis, the supervised learning being one of (i) a multivariable linear, polynomial, step, lasso, ridge, kernel, or nonlinear regression method, (ii) a support vector regression (SVR) method, (iii) a Gaussian process regression (GPR) method, (iv) a decision tree (DT) method, (ν) a random forests (RF) method, and (vi) an artificial neural network (ANN).

72. The system of claim 65, wherein the functionality landscape further includes an uncertainty value as a function of position within the latent space, the uncertainty value representing an uncertainty that has been estimated for how well the functionality landscape approximates the measured values.

73. The system of claim 41, wherein the processing circuitry is further configured to select some of the new candidate amino acid sequences for the subsequent iteration to correspond with regions in the latent space having larger uncertainty values than other regions such that, in the subsequent iteration, measured values corresponding to the some of the candidate amino acid sequences will reduce the larger uncertainty values due to increased sampling in the regions of the larger uncertainty values.

74. The system of claim 41, wherein the assay system is further configured to measure the values of the candidate proteins using at least one of (i) an assay that measures growth rate as an indicia of the desired functionality, (ii) an assay that measures gene expression as an indicia of the desired functionality, and (iii) an assay that uses microfluidics and fluorescence to measure gene expression or activity as indicia of the desired functionality.

75. The system of claim 41, wherein the gene synthesis system is further configured to synthesize the candidate genes using polymerase cycling/chain assembly (PCA) in which oligonucleotides (oligos) having overlapping extensions are provided in a solution, wherein the oligos are cycled through a series of temperatures whereby the oligos are combined into larger oligos by steps of (i) denaturing the oligos, (ii) annealing the overlapping extensions, and (iii) extending non-overlapping extensions.

76. The system of claim 41, wherein the processing circuitry is further configured to perform the iterative loop such that a parameter of the one or more assays is evolved from a starting value to a final value, such that during a first iteration the candidate genes exhibit the desired functionality when measured at the starting value, but not when measured at the final value, and during a final iteration the candidate genes exhibit the desired functionality when measured at the final value.

77. The system of claim 76, wherein the parameter of the one or more assays is one of (i) a temperature, (ii) a pressure, (iii) a light condition, (iv) a pH value, and (ν) a concentration of a substance in a medium used for the one or more assays.

78. The system of claim 76, wherein the parameter of the one or more assays is to evaluate the candidate amino acid sequences with respect to a combination of internal phenotypes and external environmental conditions.

79. A non-transitory computer readable storage medium including executable instructions, wherein the instructions, when executed by circuitry, cause the circuitry to perform a method comprising steps of:
determining candidate amino acid sequences of synthetic proteins using a machine-learning model that has been trained to learn implicit patterns in a training dataset of proteins, the machine-learning model expressing the learned implicit patterns, and
performing an iterative loop, wherein each iteration of the loop comprises determining candidate genetic sequences based on the candidate amino acid sequences,
sending to a gene synthesis system, the candidate genetic sequences to be synthesized into candidate genes to produce candidate proteins,
receiving from an assay system, measured values generated by measuring the candidate proteins using one or more assays, and
when one or more stopping criteria of the iterative loop have not been satisfied, calculating a fitness function from the measured values, and selecting, using a combination of the fitness function together with the machine-learning model, additional candidate amino acid sequences for a subsequent iteration.

80. A method of designing sequence-defined molecules having a desired functionality, the method comprising:
determining candidate sequences of molecules, which are sequence defined, the candidate sequences being generated using a machine-learning model that has been trained to learn implicit patterns in a training dataset of sequence-defined molecules, the machine-learning model expressing the learned implicit patterns; and
performing an iterative loop, wherein each iteration of the loop comprises
synthesizing candidate sequences corresponding to the candidate molecules,
evaluating a degree to which the candidate molecules respectively exhibit a desired functionality by measuring values of the candidate molecules using one or more assays, and
when one or more stopping criteria of the iterative loop have not been satisfied, calculating a fitness function from the measured values, and selecting, using a combination of the fitness function together with the machine-learning model, additional candidate sequences for a subsequent iteration.

81. The method of claim 80, wherein the step of performing the iterative loop further comprises updating, when one or more stopping criteria have not been satisfied, the machine-learning model based on an updated training dataset of molecules that includes sequences of the candidate molecules, and selecting the additional candidate sequences for the subsequent iteration using a combination of the fitness function together with the machine-learning model after having been updated based on the updated training dataset.

82. The method of claim 80, wherein the molecule is a DNA molecule and the sequences are sequences of nucleotides.

83. The method of claim 80, wherein the molecule is a RNA molecule and the sequences are sequences of nucleotides.

84. The method of claim 80, wherein the molecule is a polymer and the sequences are sequences of chemical monomers.

85. The method of claim 1, wherein the candidate proteins comprise one or more of an antibody, an enzyme, a hormone, a cytokine, growth factor, clotting factor, anticoagulation factor, albumin, antigen, an adjuvant, a transcription factor, or a cellular receptor.

86. The method of claim 1, wherein the candidate proteins are provided for selected binding to one or more other molecules.

87. The method of claim 1, wherein the candidate proteins are provided to catalyze one or more chemical reactions.

88. The method of claim 1, wherein the candidate proteins are provided for long-range signaling.

89. The method of claim 1 further comprising generating or manufacturing an end product based on the candidate proteins.

90. The method of claim 1, wherein one or more cells are produced from the candidate proteins.

91. The method of claim 90, wherein the cells produced from the candidate proteins are directed to or placed in one or more bins.

92. The method of claim 1, wherein the candidate proteins are determined by high-throughput functional screening.

93. The method of claim 92, wherein the high-throughput functional screening is implemented by a micro-fluidics apparatus that measures the fluorescence of cells corresponding to the candidate proteins.
